(19) United States
(12) Patent Application Publication
(10) Pub. No.: US 2022/0325239 A1
(43) Pub. Date: Oct. 13, 2022

(54) CELL CULTURE

(71) Applicant: AGENCY FOR SCIENCE, TECHNOLOGY AND RESEARCH, Connexis (SG)

(72) Inventors: Iwona SZCZERBINSKA, Singapore (SG); Kevin Andrew Uy GONZALES, Singapore (SG); Huck Hui NG, Singapore (SG); Yun Shen CHAN, Singapore (SG)

(21) Appl. No.: 17/620,277

(22) PCT Filed: Jun. 18, 2020
(86) PCT No.: PCT/SG2020/050339

§ 371 Date: Dec. 17, 2021

(30) Foreign Application Priority Data
Jun. 18, 2019 (SG) 10201905610Y

(51) Int. Cl. C12N 5/073; C12N 5/00

(52) U.S. Cl. CPC C12N 5/0603; C12N 5/0018; C12N 2501/727

(57) ABSTRACT

We describe a cell culture medium comprising a basal medium supplemented with a CDK1/2/9 inhibitor and a Bcr-Abl/Src kinase inhibitor. The CDK1/2/9 inhibitor may comprise AZD5438 and the Bcr-Abl/Src kinase inhibitor may comprise Dasatinib. The cell culture medium may be capable of maintaining or increasing pluripotency in a cell cultured in the cell culture medium in the absence of co-culture such as feeder cells.

We describe the use of such a medium for feeder-free culture of a naïve pluripotent stem cell as well as re-programming of a primed pluripotent stem cell into a naïve pluripotent stem cell.

### FIELD

This invention relates to the fields of medicine, cell biology, molecular biology and genetics.

### BACKGROUND

Mammalian embryonic development occurs via systematic and dynamic transitions through multiple sequential stages (Zernicka-Goetz et al., 2009), starting with the establishment of totipotency upon fertilization of the oocyte.

Totipotency in cells of the early embryo is transient and is lost when cells undergo their first cell fate specification to 2 lineages: extra-embryonic trophectoderm and pluripotent cells that will form the inner cell mass (ICM) and later the epiblast (Gardner, 1998; Gardner and Beddington, 1988; Zernicka-Goetz et al., 2009).

Pluripotent cells have the ability to contribute to all the tissues of the embryo proper, and thus all the cells of an adult, but no longer to extra-embryonic lineages (Nichols and Smith, 2009). Pluripotency of the epiblast is retained after implantation for a short period of time until cells undergo gastrulation and are specified into definitive endoderm, ectoderm and mesoderm as well as the germline (Gardner and Beddington, 1988).

The transition from the pre-implantation to post-implantation embryo triggers fundamental molecular changes in pluripotent cells. These distinct states of pluripotency can now be captured in vitro as naïve and primed states (Nichols and Smith, 2009), respectively, in both mouse (Brons et al., 2007; Evans and Kaufman, 1981; Martin, 1981; Tesar et al., 2007; Ying et al., 2008) and human pluripotent stem cell cultures (Chan et al., 2013; Gafni et al., 2013; Guo et al., 2017; Reubinoff et al., 2000; Takashima et al., 2014; Theunissen et al., 2014; Thomson et al., 1998; Ware et al., 2014).

Among these states, naïve culture of human embryonic stem cells (hESCs) is the most recently established. Formulations from various groups (Chan et al., 2013; Gafni et al., 2013; Takashima et al., 2014; Theunissen et al., 2014; Ware et al., 2014) largely overlap in terms of signalling pathways targeted by either small molecules or ectopic transcription factors, following the rationale of targeting pathways important for mouse naïve ESCs (Weinberger et al., 2016). While the molecular machinery was extensively studied for human primed, mouse primed and mouse naïve pluripotency states, the regulatory pathways governing the human naïve state remain to be dissected. This endeavour is crucial since (1) naïve hESCs serve as a useful in vitro model of early human development, which is practically and ethically challenging to study in vivo, (2) naïve cultures are more favourable than primed cultures in certain biological aspects; for example, the latter exhibits higher heterogeneity and more variability during multi-lineage differentiation (Nishizawa et al., 2016), and (3) it has been put forward that certain small molecules act differently in mouse and human pluripotent states (Ware, 2017; Weinberger et al., 2016).

Consistent with different signalling requirements, naïve cells are molecularly distinct from primed conventional human pluripotent cultures. They express naïve-specific transcription factors such as KLF4, KLF5, DPPA3, DPPA5, express higher levels of NANOG, display nuclear-specific localization of TFE3, and preferentially utilize the distal POU5F1 enhancer (Betschinger et al., 2013; Theunissen et al., 2016; Theunissen et al., 2014). These characteristics and their overall transcriptome closely resemble the in vivo ICM of human pre-implantation blastocyst (Theunissen et al., 2016).

Notably, the naïve and primed pluripotent states are each associated with a distinct repertoire of expressed transposons, robustly reflecting profiles of their counterparts in vivo (Goke et al., 2015; Theunissen et al., 2016). For example, primed hESCs are maintained by expression of HERVH driven by the LTR7 element (Lu et al., 2014), while naïve hESCs are marked by activity of the LTR7Y elements (Goke et al., 2015; Theunissen et al., 2016) as well as expression of HERVK driven by LTR5_Hs (Grow et al., 2015; Theunissen et al., 2016). The high specificity of ERV promoters, especially throughout the course of embryonic development (Goke et al., 2015), provides a unique approach for identification of cell states beyond existing in vitro models.

As described above, the distinct states of pluripotency in the pre- and post-implantation embryo may be captured in vitro as naïve and primed pluripotent stem cell cultures, respectively. The study and application of the naïve state however remains hampered, particularly in human, partially due to current culture protocols relying on extraneous undefined factors such as feeders. Thus, a major hurdle for studying the human naïve state is that, unlike mouse naïve and human primed states, its establishment and/or maintenance remains dependent on feeders.

A defined feeder-free culture condition for the in vitro counterpart of human pre-implantation blastocyst will ease the mechanistic dissection of naïve identity and facilitate the use of these cells in clinics.

### SUMMARY

We provide, according to the invention, cell culture media, methods of culturing cells and cells, as set out in the claims. Preferred embodiments are set out in the claims and are described in the description.

According to a 1^{st} aspect of the present invention, we provide a cell culture medium comprising a CDK1/2/9 inhibitor and a Bcr-Abl/Src kinase inhibitor.

The CDK1/2/9 inhibitor may comprise AZD5438 (4-[2-Methyl-1-(1-methylethyl)-1H-imidazol-5-yl]-N-[4-(methylsulfonyl)phenyl]-2-pyrimidinamine, AZD).

The Bcr-Abl/Src kinase inhibitor may comprise Dasatinib (N-(2-chloro-6-methylphenyl)-2-[[6-[4-(2-hydroxyethyl)-1-piperazinyl]-2-methyl-4-pyrimidinyl]amino]-5-thiazole carboxamide monohydrate, DASA).

The cell culture medium may comprise AZD5438 and Dasatinib.

The AZD5438 may be at a concentration of 0.1 μM or more, such as 1 μM to 0.5 μM, preferably 0.1 μM.

The Dasatinib may be at a concentration of 0.1 μM or more, such as 1 μM to 0.5 μM, preferably 0.1 μM.

The cell culture medium may comprise SB590885 ((NE)-N-[5-[2-[4-[2-(dimethylamino) ethoxy]phenyl]-5-pyridin-4-yl-1H-imidazol-4-yl]-2,3-dihydroinden-1-ylidene]hydroxylamine). The cell culture medium may comprise 0.1 to 2.5 μM, preferably 0.5 μM of SB590885.

The cell culture medium may comprise PD0325901 (N-[(2R)-2,3-dihydroxypropoxy]-3,4-difluoro-2-(2-fluoro-4-iodoanilino)benzamide). The cell culture medium may comprise 0.2 to 10 μM, preferably 1 μM of PD0325901.

The cell culture medium may comprise Y-27632 (4-[(1R)-1-aminoethyl]-N-pyridin-4-ylcyclohexane-1-carboxamide). The cell culture medium may comprise 5 to 20 μM, preferably 10 μM of Y-27632.

The cell culture medium may comprise 5 to 20 μg/ml of recombinant human LIF (UniProtKB-P15018)0.2 to 10 μM, preferably 1 μM of PD0325901; 0.1 to 2.5 μM, preferably 0.5 μM of SB590885; 0.1 to 2.5 μM, preferably 1 μM of WH4-023; 5 to 20 μM, preferably 10 μM of Y-27632; and 5 to 20 ng/ml, preferably 10 ng/ml of Activin A (UniProtKB-P08476).

The cell culture medium may comprise DMEM/F12 (Invitrogen; 11320), Neurobasal (Invitrogen; 21103), N2 supplement (Invitrogen; 17502048) (100× dilution), B27 supplement (Invitrogen; 17504044) (50× dilution), 2 mM L-glutamine, 1% non-essential amino acids, 0.1 mM β-mercaptoethanol, 1% penicillin-streptomycin, 50 μg/ml BSA, supplemented with 10 μg/mL recombinant human LIF, 1 μM PD0325901, 0.5 μM SB590885, 1 μM WH4-023, 10 μμM Y-27632 and 10 ng/mL Activin A.

The cell culture medium may comprise a 1:1 ratio of F12 DMEM (STEMCELL Technologies) and Neurobasal media (Gibco), 1× N2 supplement (Gibco) and 1× B2 supplement (Gibco), 1× L-Glutamine (Gibco), 1× Non-essential amino acids (Gibco), 0.1 mM of B-mercaptoethanol (Sigma) and 62.5 ng/ml of bovine serum albumin (BSA, Sigma).

The cell culture medium may be capable of maintaining or increasing pluripotency in a cell cultured in the cell culture medium. It may do so in the absence of co-culture such as feeder cells.

A cell cultured in the cell culture medium may express a naïve pluripotent stem cell marker. The naïve pluripotent stem cell marker may comprise CD130 (Gene ID: 3572). The naïve pluripotent stem cell marker may comprise CD75 (Gene ID: 6480). The naïve pluripotent stem cell marker may comprise DNMT3L (Gene ID: 29947). The naïve pluripotent stem cell marker may comprise DPPA5 (Gene ID: 340168). The naïve pluripotent stem cell marker may comprise

KLF5 (Gene ID: 688). The naïve pluripotent stem cell marker may comprise TFCP2L1 (Gene ID: 29842). The naïve pluripotent stem cell marker may comprise KLF4 (Gene ID: 9314). The naïve pluripotent stem cell marker may comprise DPPA3 (Gene ID: 359787). The naïve pluripotent stem cell marker may comprise NANOG (Gene ID: 79923). The naïve pluripotent stem cell marker may comprise KLF17 (Gene ID: 128209). The naïve pluripotent stem cell marker may comprise POU5F1 (Gene ID: 5460). The naïve pluripotent stem cell marker may comprise PRDM14 (Gene ID: 63978).

The cell culture medium may be capable of maintaining or increasing pluripotency in a cell cultured for 5 or more passages, such as 8 or more passages.

The cell culture medium may be capable of decreasing the expression of a primed pluripotent stem cell marker such as ZIC2 (Gene ID: 7546) and B3GAT1 (Gene ID: 27087) in a cell cultured in the cell culture medium.

There is provided, according to a 2^{nd} aspect of the present invention, a method of culturing a cell in a cell culture medium according to the 1^{st} aspect of the invention.

The method may be capable of maintaining or increasing the expression of a naïve pluripotent stem cell marker in the cell.

The method may be capable of such that it does not include or require co-culture with feeder cells.

The method may comprise culturing the cell for 5 or more passages, such as 8 or more passages.

The method may comprise culturing a naïve pluripotent stem cell, preferably a mammalian naïve pluripotent stem cell, such as a human naïve pluripotent stem cell.

The method may be capable of maintaining the naïve pluripotent stem cell in a naïve state. The method may be capable of maintaining the survival of a naïve pluripotent stem cell preferably after at least 5 passages, preferably after at least 8 passages.

The cell may comprise a primed pluripotent stem cell. The cell may comprise a mammalian primed pluripotent stem cell. The cell may comprise a human primed pluripotent stem cell The method may be such that it re-programs the primed pluripotent stem cell into a naïve pluripotent stem cell.

The cell may comprise a somatic cell. The cell may comprise a mammalian somatic cell. The cell may comprise a human somatic cell. The method may be such that it re-programs the somatic cell into a naïve pluripotent stem cell. The method may be such that it further comprises up-regulating the expression of Oct4 (Pou5f1), Sox2, Klf4 and c-Myc in the somatic cell.

We provide, according to a 3^{rd} aspect of the present invention, a method of propagation of a naïve pluripotent stem cell, the method comprising culturing the naïve pluripotent stem cell in a cell culture medium set out above.

As a 4^{th} aspect of the present invention, there is provided a method of re-programming a somatic cell or a primed pluripotent stem cell into a naïve pluripotent stem cell, the method comprising culturing the primed pluripotent stem cell in a cell culture medium set out above.

We provide, according to a 5^{th} aspect of the present invention, a cell cultured in a cell culture medium set out above.

The present invention, in a 6^{th} aspect, provides a cell produced by a method set out above.

The practice of this invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford

University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual. Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory,

ISBN 0-87969-630-3. Each of these general texts is herein incorporated by reference.

### BRIEF DESCRIPTION OF THE FIGURES

FIGS. 1A to 1D are diagrams showing a small molecule screen for feeder-free maintenance of naïve hESCs, see also FIG. 8.

FIG. 1A is a drawing showing a schematic of high-throughput screen performed to identify compounds supporting feeder-free culture of naïve hESCs. LTR7Y-zsGreen reporter cells cultured in 3 iL condition were seeded without feeders and then subjected to treatment with chemical libraries comprising 622 compounds. Dot plot presents mean z-scores for LTR7Y-zsGreen intensity results from the screen. The gray line indicates a stringent cut-off of z-scores >2, as primed cells freshly adapted to 3iL condition (green) mostly placed around this score Small molecules achieving this cut-off in at least 2 replicates were considered as hits (blue). Other samples (orange) and DMSO controls (red) did not pass this cut-off. Full list of scores in Table E1.

FIG. 1B is a diagram showing a summary table with hits from the small molecule screen including names, library, concentrations, mean LTR7Y-zsGreen z-scores and known pathway(s) targeted by each compound. *=compounds targeting pathways not previously demonstrated to play a role in establishment/maintenance of naïve pluripotency.

FIG. 1C is a diagram showing representative fluorescent microscopy images of LTR7Y-zsGreen cells after treatment with small molecule hits. Scale bar=50 μm.

FIG. 1D is a diagram showing a FACS quantification of LTR7Y-zsGreen signal after treatment with Dasatinib, Crenolanib, AZD5438 and at various concentrations.

FIGS. 2A to 2F are diagrams showing optimisation and establishment of FINE culture conditions.

FIG. 2A and FIG. 2B are diagrams showing gene expression analysis for naïve markers in (A) 3iL cultured cells and (B) 4 iLA cultured cells supplemented with various small molecules. Mean±SD of three independent experiments. RNA was collected after 6 days (3iL) or 12 days (4 iLA) in culture without feeders.

FIG. 2C is a diagram showing relative survival of hESC culture under 4 iLA supplemented with different chemical combinations conditions (C1-C21) over 9 passages without feeders. 4 iLA was included as control. When cells appear highly differentiated morphologically or when very little cells remain after passaging, the condition is dropped off; only cells cultured in C18-C21-4 iLA medium supplemented with AZD5438 (AZD) and Dasatinib (DASA) (in green) remain after 9 passages. Detailed conditions are shown in Table E2.

FIG. 2D is a diagram showing a heatmap presenting gene expression of naïve pluripotency associated markers in cells at passage 4 during adaptation to naïve feeder-free conditions (C1-C21). Mean of two biological replicates is shown. Euclidean distance from 4 iLA+feeder across all the genes tested was calculated for each condition and represented as the bar chart. C19 showed highest correlation (shortest distance) with 4 iLA+feeder condition (in green).

FIG. 2E is a diagram showing a schematic showing the process of adapting primed hESCs into FINE.

FIG. 2F is a diagram showing gene expression in hESCs throughout the course of adaptation from mTeSR1 to FINE up to passage 5. Mean±SD of two independent experiments.

FIGS. 3A to 3H are diagrams showing FINE cells display hallmarks of naïve pluripotency, see also FIG. 9 and FIG. 10.

FIG. 3A is a diagram showing brightfield images of hESCs cultured in 4 iLA (with and without feeders) and FINE at passage 8. Scale bar=50 μm.

FIG. 3B is a diagram showing expression of blastocyst-associated transcripts in hESCs cultured under mTeSR1, 4 iLA+feeder and FINE conditions. Mean±SD of three independent experiments.

FIG. 3C is a diagram showing immunofluorescence staining of pluripotency and blastocyst-associated proteins in hESCs under mTeSR1 and FINE conditions. Scale bar=50 μm.

FIG. 3D is a diagram showing FACS quantification of hESCs expressing naïve surface markers under mTeSR1, 4 iLA+feeder and FINE conditions.

FIG. 3E is a diagram showing qPCR analysis of LTR7Y and HERVH transcripts in hESCs cultured under mTeSR1, 4 iLA+feeder and FINE conditions. Mean±SD of three independent experiments.

FIG. 3F is a diagram showing measurement of proliferation rate of cells cultured in FINE conditions. 120,000 cells were seeded (D0 in white) and cell count was performed 4 days post-seeding (D4 in gray) at passage 8 to 12. Mean±SD of three independent experiments.

FIG. 3G is a diagram showing representative RNA FISH images detecting HUWEl (subject of X chromosome inactivation) and XACT control (escaping X chromosome inactivation) in mTeSR1, 4 iLA +feeders and FINE cells. Scale bar=10 μm.

FIG. 3H is a diagram showing immunofluorescence staining of H3K9me3 in hESCs under mTeSR1 and FINE conditions (left). Scale bar=50 μm. Intensity of H3K9me3 was quantified through a line (red) randomly drawn across images (right).

FIGS. 4A to 4E are diagrams showing FINE cells are dependent on both Dasatinib and AZD5438.

FIG. 4A is a diagram showing brightfield and immunofluorescence staining of KLF4, TFE3 and KLF17 in FINE culture after withdrawal of AZD, Dasa or both for 3 passages. Scale bar=50 μm.

FIG. 4B is a diagram showing quantification of fraction of nuclei positive for naïve-associated transcription factors in hESCs cultured in FINE after withdrawal of AZD, Dasa or both for 3 passages. Mean±SD of three independent experiments.

FIG. 4C is a diagram showing expression of blastocyst-associated transcripts in hESCs cultured in FINE after withdrawal of AZD, Dasa or both for 2 passages. Mean±SD of three independent qPCR experiments.

FIG. 4D and FIG. 4E are diagrams showing expression of blastocyst-associated transcripts in hESCs cultured in FINE after (D) replacement of Dasa with other Src and Bcr-Abl inhibitors or (E) replacement of AZD with Dinaciclib (in H9 line) for 2 passages. Mean±SD of three independent qPCR experiments.

FIGS. 5A to 5G are diagrams showing that the transcriptomic profile of FINE resembles the in vivo pre-implantation blastocyst, see also FIG. 11.

FIG. 5A is a diagram showing PCA based on top 1000 differentially expressed genes between mTeSR1, 4 iLA+feeder and FINE cultured cells. RNA-seq was performed in biological duplicates.

FIG. 5B is a diagram showing a heatmap of top 1000 differentially expressed genes between mTeSR1, 4 iLA+feeder and FINE cultured cells. 6 main clusters were defined by dendrogram (left). Representative genes from two main clusters (naïve-specific genes, primed-specific genes) are presented in smaller heatmaps (right).

FIG. 5C is a diagram showing scatter plots showing significantly upregulated (in red) and downregulated (in blue) genes between: mTeSR vs 4 iLA+feeder, mTeSR1 vs FINE and 4 iLA+feeder vs FINE conditions. Genes not differentially expressed are presented in black.

FIG. 5D is a diagram showing correspondence between gene expression (left) or TE expression (right) between our naïve/primed ESCs and single-cell human embryonic stages from (Yan et al., 2013). For each embryonic stage, the percentage of genes/TEs with expression upregulated in FINE (green), upregulated in mTeSR1 (dark gray) or unchanged between FINE and mTESR1 (light gray), is shown. Analysis was performed following (Theunissen et al., 2016).

FIG. 5E is a diagram showing PCA plot based on the top 2540 repeat elements differentially expressed across conditions. Single cell in vivo embryonic data (Yan et al., 2013) are represented as squares, while FINE, 4 iLA+feeder and mTeSR1 from our bulk RNA-seq data are drawn as circles.

FIG. 5F is a diagram showing boxplots representing mean normalized expression of different TEs in mTeSR1, 4 iLA+feeder and FINE cultured cells.

FIG. 5G is a diagram showing percentage of members in each TE family with expression upregulated in FINE (green), upregulated in mTeSR1 (dark gray) or unchanged between FINE and mTESR1 (light gray). TE families were ranked — specific to FINE conditions on the left and specific to mTeSR1 condition on the right.

FIGS. 6A to 6D are diagrams showing global DNA methylation profile of FINE confirms equivalence to feeder-dependent naïve pluripotent hESCs.

FIG. 6A is a diagram showing per chromosome comparison of CG methylation fraction between mTeSR1, 4 iLA+feeder and FINE conditions.

FIG. 6B is a diagram showing relative methylation tracks of chromosome 4 under mTeSR1, 4 iLA+feeder and FINE conditions.

FIG. 6C is a diagram showing correlation plot of methylated sites in FINE versus either mTeSR1 and 4 iLA+feeder. Red line represents fit based on linear regression modelling (off-center best-fit indicates lower correlation); blue line is based on LOESS weighted regression modelling (curved best-fit line indicates non-linear correlation).

FIG. 6D is a diagram showing box plots (top) for CG methylation fraction at select loci representing naïve-, differentiation-, 8C- and morula-associated genes, as well as relative methylation tracks of one representative gene per group (bottom). Differential peaks are highlighted in yellow for ZSCAN4 and DNAJCJ5.

FIGS. 7A to 7D are diagrams showing FINE cells offer advantages over other naïve culture conditions, see also FIG. 12.

FIG. 7A is a diagram showing representative images (left) and FACS quantification (right) of cells in FINE and 4 iLA+feeder culture conditions after transfection with mCherry-containing plasmids gRNA 1 (targeting EGFR) and gRNA 2 (targeting STAG2). Quantification was performed after staining with an anti-CD75 antibody to account for feeders; Mean±SD of two independent experiments.

FIG. 7B is a diagram showing summary of cytogenetic analysis of H9 cells (top) under various naïve culture conditions (rows) and passage numbers (columns). Representative karyotypes at various passage numbers in FINE (bottom).

FIG. 7C is a diagram showing qPCR analysis of naïve-associated transcripts in H1 hESCs cultured under mTeSR1, RSeT and FINE conditions. Mean±SD of three independent experiments.

FIG. 7D is a diagram showing heatmap showing rlog values for expression of 8-cell- and morula-stage-associated genes in mTeSR1, 4iL+feeder and FINE cultures based on RNA-seq.

FIGS. 8A to 8M are diagrams showing validation of LTR7Y-zsGreen reporter and quality control of small molecule screen (related to FIGS. 1A to 1D).

FIG. 8A is a diagram showing gene expression analysis of pluripotency associated genes: OCT4, NANOG, SOX2 and PRDM14 in WT-H1 (parental line) and LTR7Y-zsGreen reporter line. Mean±SD of three independent experiments.

FIG. 8B is a diagram showing immunofluorescence staining of pluripotency markers: OCT4, TRA-1-60, TRA-1-81 in WT-H1 (parental line) and LTR7Y-zsGreen reporter cells. Scale bar=50 μm.

FIG. 8C is a diagram showing LTR7Y-zsGreen reporter cells give rise to teratomas consisting of cells from mesodermal, ectodermal and endodermal lineages.

FIG. 8D is a diagram showing cytogenetic analysis of LTR7Y-zsGreen reporter cells confirms normal karyotype.

FIG. 8E is a diagram showing FACS analysis of LTR7Y-zsGreen reporter cells cultured in mTeSR1 (green), 3iL (orange) and mTeSR1 supplemented with retinoic acid (RA) culture conditions.

FIG. 8F is a diagram showing microscopy images showing induction of LTR7Y-zsGreen reporter activity in 3iL with feeder and 3iL without feeder culture conditions, compared to mTeSR1. Scale bar=50 μm.

FIGS. 8G and 8H is a diagram showing gene expression analysis for (FIG. 8G) pluripotency markers and (FIG. 8H) naïve markers in 3iL culture with or without feeders. Mean ±SD of three independent experiments. RNA was collected after 6 days in culture.

FIG. 8I is a diagram showing representative heatmap for z-scores of one plate from 3iL screen. No plate layout bias is evident.

FIG. 8J is a diagram showing boxplots showing the alignment of plates after z-score normalisation for 3iL LTR7Y-zsGreen small molecule screen.

FIG. 8K is a diagram showing scatterplot showing correlation between replicates for 3iL screen. Pearson correlation values between replicates are indicated.

FIG. 8L is a diagram showing descending plot of screen samples. Inflection point (denoted by arrow) is below the chosen stringent cut-off of z-score >2. Most positive controls (primed→3 iL) are above the inflection point.

FIG. 8M is a diagram showing representative dot plot of z-scores (y axis) versus cell count (x axis) from 3 iL chemical screen. No significant correlation is observed.

FIGS. 9A to 9H are diagrams which show supplementary data relating to FIG. 3.

FIG. 9A is a diagram showing immunofluorescence staining of OCT4, NANOG, KLF4, KLF17, TFE3 and CD75 in H1 cells cultured under mTeSR1, 4 iLA+feeder and FINE+WH-4-023 conditions. Scale bar=50 μm.

FIG. 9B is a diagram showing gene expression of lineage-specific markers in H1 cell culture under mTeSR1, 4iL+feeder and FINE conditions. Mean±SD of three independent experiments.

FIG. 9C is a diagram showing side-by-side comparison of immunofluorescence staining of NANOG, KLF4 and KLF17 in H1 cells cultured under 4 iLA+feeder and FINE conditions to demonstrate heterogeneity of expression in both naïve conditions. Arrows highlight representative cells that are positive in the green channel (green arrow), red channel (red arrow), both channels (yellow arrow) or negative for both channels (white arrow). Scale bar=50 μm.

FIG. 9D is a diagram showing FINE cultured cells give rise to teratomas consisting of cells from mesodermal, ectodermal and endodermal lineages.

FIG. 9E is a diagram showing immunofluorescence staining of Ki67 proliferation marker of hESCs under mTeSR1, 4 iLA+feeder and FINE conditions. Scale bar=50 μm.

FIG. 9F is a diagram showing measurement of proliferation rate of hESCs (H1 and H9) cultured in various conditions. 120,000 cells were seeded (DO in white) and cell count was performed 4 days post-seeding (D4 in gray) at passage 8. Mean±SD of three independent experiments.

FIG. 9G is a diagram showing quantification of percentage of HUWE1+cells in hESCs under mTeSR1, 4 iLA+feeder and FINE. Only cells showing biallelic XACT were included in the analysis. N=60 cells.

FIG. 9H is a diagram showing qPCR analysis of transcripts from the X-chromosome in hESCs cultured under mTeSR1, 4 iLA+feeder and FINE conditions, to determine X activation status. Mean±SD of three independent experiments.

FIGS. 10A to 10C are diagrams showing that FINE culture is applicable to multiple human pluripotent cell lines (related to FIGS. 3A to 3H).

FIG. 10A is a diagram showing gene expression of naïve-associated genes in H9 and HES3 cell lines, and in the GM23338 iPSC line cultured under mTeSR1 and FINE conditions. Mean±SD of three independent experiments.

FIG. 10B is a diagram showing qPCR analysis of LTR7Y and HERVH in H9 and HES3 cells cultured under mTeSR1 and FINE conditions for H9, HES3 and iPSC lines. Mean±SD of three independent experiments.

FIG. 10C is a diagram showing immunofluorescence staining of OCT4, KLF4, KLF17, TFE3 and CD75 in HES3 and H9 cells cultured under mTeSR1 and FINE conditions. Scale bar =50 μm.

FIGS. 11A to 11E are diagrams which show supplementary data relating to FIG. 5.

FIG. 11A is a diagram showing hierarchical clustering based on top 1000 differentially expressed genes between mTeSR1, 4 iLA+feeder and FINE cultured cells.

FIG. 11B is a diagram showing gene ontology analysis of terms enriched in 4 iLA+feeder in comparison to FINE cultured cells and FINE enriched terms in comparison to 4 iLA+feeder cultured cells.

FIG. 11C is a diagram showing representative genes from differentially expressed genes between 4 iLA+feeder and FINE cultures presented in heatmaps grouped based on putative roles (by gene ontology).

FIG. 11D is a diagram showing PCA plot based on the top 3489 genes differentially expressed across conditions. Single cell in vivo embryonic data (Yan et al., 2013) are represented as squares, while FINE, 4 iLA+feeder and mTeSR1 from our bulk RNA-seq data are drawn as circles.

FIG. 11E is a diagram showing a heatmap of RNA-seq expression data based on top 1000 differentially expressed transposable elements between mTeSR1, 4 iLA+feeder and FINE cultured cells.

FIGS. 12A to 12E are diagrams which show supplementary data relating to FIG. 7.

FIG. 12A is a diagram showing representative FACS gating for quantification of cells in FINE and 4 iLA+feeder culture conditions after transfection with mCherry-containing plasmid gRNA 2 and staining with an anti-CD75 antibody.

FIG. 12B is a diagram showing targeting efficiency for FINE and 4 iLA+feeder determined by T7 endonuclease assay. Gel image example from cells transfected with gRNA 2 (left), and quantification of 5 replicates (right).

FIG. 12C and FIG. 12D are diagrams showing qPCR analysis of naïve-associated transcripts in (FIG. 12C) H9 hESCs cultured under mTeSR1, FINE and FINE with low PD03 conditions, and (FIG. 12D) in H1 hESCs cultured under FINE (P5 and P24) and mTeSR1. Mean ±SD of three independent experiments.

FIG. 12E is a diagram showing qPCR analysis of 8-cell-stage-associated transcripts in H1 hESCs cultured under mTeSR1, 4 iLA+feeder and FINE conditions. Mean±SD of two independent experiments.

### DETAILED DESCRIPTION

In this study, we took advantage of the specific activity of LTR7Y in naïve pluripotency as a tool for establishment of feeder-free naïve culture conditions.

We performed a high-throughput screening to identify small molecules that are able to enhance feeder-independent culture of human naïve pluripotent stem cells. Using these small molecules, we derived a novel culture media for long term culture of feeder-independent human naïve pluripotent stem cells.

Specifically, we identify the presence of a CDK1/2/9 inhibitor such as AZD5438 and a Bcr-Abl/Src kinase inhibitor such as Dasatinib as enabling feeder-free naïve cell culture.

These compounds therefore facilitate chemically-defined establishment and maintenance of human feeder-independent naïve ESCs (or FINE). The culture media may also be used to reprogramme prime pluripotent stem cells to naïve pluripotent stem cells.

In summary, by combining a sensitive stage-specific ERV reporter with a high-throughput chemical screen, we identified novel molecules that we utilized to create human feeder-independent naïve ESCs (or FINE).

The expression profile in genic and repetitive elements of FINE cells resembles the 8-cell-to-morula stage in vivo, and only differs from feeder-dependent naïve cells in genes involved in cell-cell/cell-matrix interactions.

FINE cells offer several technical advantages such as increased amenability to transfection and a longer period of genomic stability compared to feeder-dependent cells. Thus, FINE cells will serve as an accessible and useful system for scientific and translational applications of naïve pluripotent stem cells.

The feeder-independent and chemically defined culture system will also be of great interest to pluripotent stem cells researchers.

### Cell Culture Medium

We describe a cell culture medium.

The cell culture medium comprises a CDK1/2/9 inhibitor and a Bcr-Abl/Src kinase inhibitor, such as AZD5438 and Dasatinib. These are described in further detail below.

The cell culture medium may be used to expand a population of pluripotent stem cells. We therefore describe a composition comprising: (a) a cell culture medium described here; and (b) pluripotent stem cells.

The cell culture medium is capable of growth and maintenance of pluripotent cells, without the requirement for feeder cells.

The cell culture medium is capable of maintaining pluripotency over an extended period of time, over multiple passages.

The cell culture medium may be capable of expanding a population of stem cells in a pluripotent, undifferentiated and proliferative state for at least 3 passages under appropriate conditions. Stem cells are considered to be in a pluripotent, undifferentiated and proliferative state if they exhibit certain characteristics as known in the art and also described elsewhere in this document. Appropriate conditions may be selected by a person skilled in the art from those normally used for pluripotent stem cell culture.

For example, a culture medium may be capable of expanding a population of stem cells in a pluripotent, undifferentiated and proliferative state for at least 3, at least 4, at least 5, at least 6, at least 7, at least 8, at least 9, at least 10, at least 20, at least 30, at least 40, at least 50, at least 60, at least 70, at least 80, at least 90 or at least 100, passages under appropriate conditions.

The culture medium may be capable of expanding a population of pluripotent stem cells in a pluripotent, undifferentiated and proliferative state for more than 3 passages, more than 4 passages, more than 5 passages, more than 10 passages, more than 15 passages, more than 20 passages, more than 25 passages, more than 30 passages, more than 40 passages, more than 50 passages, or more than 100 passages.

Accordingly, the stem cells may be cultured in a pluripotent, undifferentiated and proliferative state for at least 3, at least 4, at least 5, at least 6, at least 7, at least 8, at least 9, at least 10, at least 15, at least 20, at least 25, at least 30, at least 40, at least 50, at least 60, at least 70, at least 80, at least 90 or at least 100, passages under appropriate conditions.

A cell culture medium as disclosed in this document may be capable of expanding at least 2, at least 3, at least 4, at least 5, at least 6, at least 7, at least 8, at least 9, or at least 10 different pluripotent stem cell lines (e.g. different human ESC lines) in a pluripotent, undifferentiated and proliferative state for multiple passages under appropriate conditions. For example, a culture medium may be capable of expanding at least the H1 (WA-01), HES3 (ES-03), H9 (WA-09) and/or iPSCs (GM23338) cell lines in a pluripotent, undifferentiated and proliferative state for at least 3, at least 4, at least 5, at least 6, at least 7, at least 8, at least 9, or at least 10, passages under appropriate conditions.

The cull culture medium may be used for culture of different cell types. Cells grown in the cell culture medium express one or more characteristics of naïve pluripotent cells. The cell culture medium may therefore be used for the reprogramming of cells, such as primed pluripotent cells, into naïve pluripotent cells.

The cell culture medium may comprise a CDK1/2/9 inhibitor, such as AZD5438. The cell culture medium may also comprise a Bcr-Abl/Src kinase inhibitor, such as Dasatinib.

The cell culture medium may comprise AZD5438 and Dasatinib at any suitable concentration. The AZD5438 and Dasatinib may each independently be present at a concentration of 0.1 μM or more, such as 1μM to 0.504, such as 0.104 or 0.2 μM each.

The cell culture medium may also contain another compound. The cell culture medium may contain a plurality of other compounds.

The other compound or compounds may be present at any suitable concentration, such as 0.1 μM or more, 0.2 μM or more, 0.3 μM or more, 0.4 μM or more, 0.5 μM or more, 0.6 μM or more, 0.7 μM or more, 0.8 μM or more, 0.9 μM or more, 1.0 μM or more, 1.1 μM or more, 1.2 μM or more, 1.3 μM or more, 1.4 μM or more, 1.5 μM or more, 1.6 μM or more, 1.7 μM or more, 1.8 μM or more, 1.9 μM or more, 2.0 μM or more, 2.1 μM or more, 2.2 μM or more, 2.3 μM or more, 2.4 μM or more, 2.5 μM or more, 2.6 μM or more, 2.7 μM or more, 2.8 μM or more, 2.9 μM or more, 3.0 μM or more, 3.1 μM or more, 3.2 μM or more, 3.3 μM or more, 3.4 μM or more, 3.5 μM or more, 3.6 μM or more, 3.7 μM or more, 3.8 μM or more, 3.9 μM or more, 4.0 μM or more, 4.1 μM or more, 4.2 μM or more, 4.3 μM or more, 4.4 μM or more, 4.5 μM or more, 4.6 μM or more, 4.7 μM or more, 4.8 μM or more or 4.9 μM or more.

Higher concentrations of the compound or compounds are also possible, such as 5 μM or more, 6 μM or more, 7 μM or more, 8 μM or more, 9 μM or more, 10 μM or more, 11 μM or more, 12 μM or more, 13 μM or more, 14 μM or more, 15 μM or more, 16 μM or more, 17 μM or more, 18 μM or more, 19 μM or more or 20 μM or more.

It will be understood that, where the cell culture medium contains more than one additional compound, that the individual compounds may be present at different concentrations.

For example, the cell culture medium may comprise SB590885 ((NE)-N-[5-[2-[4-[2-(dimethylamino) ethoxy]phenyl]-5-pyridin-4-yl-1H-imidazol-4-yl]-2,3-dihydroinden-1-ylidene]hydroxylamine). The SB590885 may be present at a concentration of 0.1 to 2.5 μM, such as 0.5 μM of SB590885.

The cell culture medium may also comprise PD0325901 (N-[(2R)-2,3-dihydroxypropoxy]-3,4-difluoro-2-(2-fluoro-4-iodoanilino)benzamide). The PD0325901 may be present at a concentration of 0.2 to 10 μM, such as 1 μM of PD0325901.

The cell culture medium may also comprise Y-27632 (4-[(1R)-1-aminoethyl]-N-pyridin-4-ylcyclohexane-1-carboxamide). The Y-27632 may be present at a concentration of 5 to 20 μM, such as 10 μM of Y-27632.

The cell culture medium may also comprise recombinant human LIF (UniProtKB-P15018). The recombinant human LIF (UniProtKB-P15018) may be present at a concentration of 5 to 20 μg/ml.

The cell culture medium may also comprise PD0325901. The PD0325901 may be present at a concentration of 0.2 to 10 μM, such as 1 μM.

The cell culture medium may also comprise SB590885. The SB590885 may be present at a concentration of 0.1 to 2.5 μM, such as 0.5 μM.

The cell culture medium may also comprise WH4-023. The WH4-023 may be present at a concentration of 0.1 to 2.5 μM, such as 104.

The cell culture medium may also comprise Y-27632. The Y-27632 may be present at a concentration of 5 to 20 μM, such as 1004.

The cell culture medium may also comprise Activin A (UniProtKB-P08476). The Activin A (UniProtKB-P08476) may be present at a concentration of 5 to 20 ng/ml, such as 10 ng/ml.

The cell culture medium may be made up from a basal medium.

Basal media contain amino acids, glucose, and ions (calcium, magnesium, potassium, sodium, and phosphate) essential for cell survival and growth. Suitable basal media are known in the art and are available commercially.

The basal medium may comprise for example Dulbecco's Modified Eagle's Medium (DMEM or DMEM F12) and alpha-Minimum Essentials Medium (a-MEM).

For this purpose, a suitable amount of a CDK1/2/9 inhibitor such as AZD5438 and a Bcr-Abl/Src kinase inhibitor such as Dasatinib are added to the basal medium to achieve the required concentration.

The cell culture medium may comprise serum, such as foetal bovine serum, or be chosen to be serum free.

An example of a composition of a cell culture medium suitable for use in the methods and compositions described here consists of basal media and supplements.

The basal media may comprise
1:1 ratio of F12 DMEM and Neurobasal (Gibco) media
1× N2 supplement (Gibco)
1× B2 supplement (Gibco)
1× L-Glutamine (Gibco)
1× Non-essential amino acids (Gibco)
0.1 mM of B-mercaptoethanol (Sigma)
62.5 ng/ml of BSA (Sigma)

The DMEM/F12 and Neurobasal may be present at any suitable ratio, such as 1:2, 1:3, 1:4 or 1:5, or 5:1, 4:1, 3:1 or 2:1. The DMEM/F12 and Neurobasal may be present at 1:1 ratio, for example.

The basal media may be supplemented with
0.1 μM of Dasatinib (Selleckchem)
0.1 μM AZD5438 (TOCRIS)
0.1 04 SB590885 (Sigma)
0.1 04 of PD0325901 (Sigma)
10 μM of Y-27632 (STEMCELL Technologies)
20 ng/ml of human recombinant LIF (Peprotech)
20 ng/ml of Activin A (STEMCELL Technologies)
8 ng/ml of bFGF (Gibco)
The basal media and supplement combination described above (DMEM/F12 and Neurobasal in 1:1 ratio) may be referred to as FINE media.

### CDK1/2/9 Inhibitor

The cell culture medium may comprise a CDK1/2/9 inhibitor.

As used in this document, a CDK1/2/9 inhibitor should be taken to be anything that is capable of inhibiting an activity of any combination of CDK1, CDK2 and CDK9 (such as each of CDK1, CDK2 and CDK9). The activity may comprise a cyclin dependent kinase activity of CDK1, CDK2 and/or CDK9, as the case may be.

Assays for kinase activity and measurement of such activity, including inhibition of kinase activity, are well known in the art.

The cell culture medium may comprise two or more, three or more, four or more, five or more, six or more, seven or more, eight or more, nine or more, ten or more, eleven or more, or twelve or more, different CDK1/2/9 inhibitors.

An example of a CDK1/2/9 inhibitor suitable for use in the cell culture medium described here is the compound AZD5438.

### AZD5438

AZD5438 (4-[2-Methyl-1-(1-methylethyl)-1H-imidazol-5-yl]-N-[4-(methylsulfonyl)phenyl]-2-pyrimidinamine), also known as AZD-5438, AZD 5438 and AZD.

AZD5438 has CAS Number 602306-29-6 , an empirical formula (Hill Notation) of C₁₈H₂₁N₅O₂S and a molecular weight of 371.46.

AZD5438 is an inhibitor of cyclin-dependent kinases 1, 2, and 9 and glycogen synthase kinase GSK-3β. AZD5438 effectively inhibits cellular CDK substrates phosphorylation and displays antiproliferation activity against a broad spectrum of human cancer cultures by inducing cell cycle arrest at G2-M, S, and Gl. Oral administration (50 mg/kg/12 h or 75 mg/kg/day) is efficacious against human tumor xenograft growth in mice in vivo.

AZD5438 is described in detail in Hazlitt et al (2018) Development of Second-Generation CDK2 Inhibitors for the Prevention of Cisplatin-Induced Hearing Loss, J Med Chem. 2018 Sep 13; 61(17): 7700-7709 and Byth et al (2009), AZD5438, a potent oral inhibitor of cyclin-dependent kinases 1, 2, and 9, leads to pharmacodynamic changes and potent antitumor effects in human tumor xenografts. Mol Cancer Ther (8) (7) 1856-1866.

AZD5438 is available commercially from a number of sources, including Sigma-Aldrich under catalogue number SML1855, Selleckchem under catalogue number 52621 and Tocris under catalogue number 3968.

The cell culture medium described here may contain AZD5438 at any suitable concentration, for example, 0.1 μM or more, such as 0.1 μM or more, 0.2 μM or more, 0.3 μM or more, 0.4 μM or more, 0.5 μM or more, 0.6 μM or more, 0.7 μM or more, 0.8 μM or more or 0.9 μM or more, 1.1 μM or more, 1.2 μM or more, 1.3 μM or more, 1.4 μM or more, 1.5 μM or more, 1.6 μM or more, 1.7 μM or more, 1.8 μM or more, 1.9 μM or more or 2 μM or more.

The cell culture medium described here may contain AZD5438 at a concentration of 2.1 μM or more, 2.2 μM or more, 2.3 μM or more, 2.4 μM or more, 2.5 μM or more, 2.6 μM or more, 2.7 μM or more, 2.8 μM or more, 2.9 μM or more or 3.0 μM or more.

The cell culture medium described here may comprise 1 mM to 400 mM, 10 mM to 390 mM, 20 mM to 380 mM, 30 mM to 370 mM, 40 mM to 360 mM, 50 mM to 350 mM, 60 mM to 340 mM, 70 mM to 330 mM, 80 mM to 320 mM, 90 mM to 310 mM, 100 mM to 300 mM, 110 mM to 290 mM, 120 mM to 280 mM, 130 mM to 270 mM, 140 mM to 260 mM, 150 mM to 250 mM, 160 mM to 240 mM, 170 mM to 230 mM, 180 mM to 220 mM, 190 mM to 210 mM, such as 200 mM of AZD5438.

It will be understood that AZD5438 may be derivatised through means known in the art, and that AZD5438 derivatives may be used in addition to, or in place of, AZD5438, in the cell culture media described here. For example, derivates of AZD5438 are known from Diao et al (2019), Discovery of novel pyrimidine-based benzothiazole derivatives as potent cyclin-dependent kinase 2 inhibitors with anticancer activity. European Journal of Medicinal Chemistry 179, 196-207.

Such AZD5438 derivatives include for example the compound referred to as 10 s in Diao et al (2019), which may be used in the cell culture medium described here. Hazlitt et al (2018) also describes a number of CDK inhibitors which may also be used in the cell culture medium.

### Bcr-Abl/Src Kinase Inhibitor

The cell culture medium may comprise a Bcr-Abl/Src kinase inhibitor.

As used in this document, a Bcr-Abl/Src kinase inhibitor should be taken to be anything that is capable of inhibiting an activity of any combination of Bcr-Abl kinase and Src kinase (such as each of Bcr-Abl kinase and Src kinase).

Assays for kinase activity and measurement of such activity, including inhibition of kinase activity, are well known in the art.

The cell culture medium may comprise two or more, three or more, four or more, five or more, six or more, seven or more, eight or more, nine or more, ten or more, eleven or more, or twelve or more, different Bcr-Abl/Src kinase inhibitors.

An example of a Bcr-Abl/Src kinase inhibitor suitable for use in the cell culture medium described here is the compound Dasatinib.

### Dasatinib

Dasatinib (N-(2-chloro-6-methylphenyl)-2-[[6-[4-(2-hydroxyethyl)-1-piperazinyl]-2-methyl-4-pyrimidinyl]amino]-5-thiazole carboxamide monohydrate, DASA).

Dasatinib, also known as Sprycel, BMS-354825 and BMS 354825, has CAS Number 302962-49-8, an empirical formula (Hill Notation) of C₂₂H₂₆ClN₇O₂S and a molecular weight of 488 g/mol. It is a thiazole carboximide derivative, structurally related to imatinib.

Dasatinib is an orally potent, bioavailable inhibitor of BCR-ABL1. It was approved by the US Food and Drug Administration (FDA) in 2006 for the treatment of imatinib-resistant and -intolerant adults with CML-CP and advanced disease as well as Ph-positive acute lymphoblastic leukemia.

In addition to blocking BCR-ABLJ kinase activity, dasatinib inhibits a distinct spectrum of oncogenic kinases, including Src family kinases (SFKs), c-Kit, platelet-derived growth factor-receptor (PDGFR), and ephrin-A receptor.

Dasatinib is described in Dasatinib: BMS 354825, Drugs R D. 2006;7(2):129-32.

Dasatanib is available commercially from a number of vendors, for example AK Scientific, Inc. (AKSCI) (Catalogue Number: 2359AH), BioCrick (Catalogue Number: BCC1281), Boc Sciences (Catalogue Number: 1132093-70-9), Norris Pharm (Catalogue Number: NSTH-D29628), Specs (Catalogue Number: AR-270/43507994), Chemenu Inc. (Catalogue Number: CM110065), AbMole Bioscience (Catalogue Number: 1701) and King Scientific (Catalogue Number: KS-0000027F).

The cell culture medium described here may contain dasatanib at any suitable concentration, for example, 0.1 μM or more, such as 0.1 μM or more, 0.2 μM or more, 0.3 μM or more, 0.4 μM or more, 0.5 μM or more, 0.6 μM or more, 0.7 μM or more, 0.8 μM or more or 0.9 μM or more, 1.1 μM or more, 1.2 μM or more, 1.3 μM or more, 1.4 μM or more, 1.5 μM or more, 1.6 μM or more, 1.7 μM or more, 1.8 μM or more, 1.9 μM or more or 2 μM or more.

The cell culture medium described here may contain dasatanib at a concentration of 2.1 μM or more, 2.2 μM or more, 2.3 μM or more, 2.4 μM or more, 2.5 μM or more, 2.6 μM or more, 2.7 μM or more, 2.8 μM or more, 2.9 μM or more or 3.0 μM or more.

The cell culture medium described here may comprise 1 mM to 400 mM, 10 mM to 390 mM, 20 mM to 380 mM, 30 mM to 370 mM, 40 mM to 360 mM, 50 mM to 350 mM, 60 mM to 340 mM, 70 mM to 330 mM, 80 mM to 320 mM, 90 mM to 310 mM, 100 mM to 300 mM, 110 mM to 290 mM, 120 mM to 280 mM, 130 mM to 270 mM, 140 mM to 260 mM, 150 mM to 250 mM, 160 mM to 240 mM, 170 mM to 230 mM, 180 mM to 220 mM, 190 mM to 210 mM, such as 200 mM of dasatanib.

It will be understood that dasatanib may be derivatised through means known in the art, and that dasatanib derivatives may be used in addition to, or in place of, dasatanib, in the cell culture media described here.

### Cell Culture Methods

The methods and compositions described here enable the culture of a cell outside the body of an organism, i.e., artificial cell culture.

The methods and compositions described here allow for the cell to be cultured in the cell culture medium without the requirement for feeder cells (i.e., feeder-cell independent culture).

The cell may comprise a stem cell. The cell may comprise an embryonic stem cell. It may comprise a pluripotent cell. The cell may comprise a primed pluripotent cell or a naïve pluripotent cell.

The cell may comprise a vertebrate cell. The cell may in particular comprise a mammalian cell, such as a rodent cell, for example a hamster, guinea pig, mouse or rat cell. The cell may comprise a sheep, chicken, llama, cow, horse, pig, camel, dog, cat, rabbit, fish, or bird cell.

The cell may comprise a primate cell, such as a human cell. Such cells may be obtained from any suitable source, as understood by a person skilled in the art.

The cell may comprise an induced pluripotent stem cell (iPSC). iPSC may be produced by means known in the art, for example by inducing expression of Myc, Oct3/4, Sox2 and Klf4, as described in Takahashi and Yamanaka (2006). Induction of pluripotent stem cells from mouse embryonic and adult fibroblast cultures by defined factors. Cell. 126 (4): 663-76.

The cell may be cultured over an extended period of time. The cell culture medium may enable the culture of a cell for 3 or more passages, such as 4 or more passages, 5 or more passages, 6 or more passages, 7 or more passages, 8 or more passages, 9 or more passages, 10 or more passages, 11 or more passages, 12 or more passages, 13 or more passages, 14 or more passages, 15 or more passages, 16 or more passages, 17 or more passages, 18 or more passages, 19 or more passages or 19 or more passages.

The cultured cell may be capable of maintaining, such as expressing, one or more characteristics of a pluripotent cell (for example a naïve pluripotent cell) during such extended culture.

The cell culture medium may be used to expand a population of pluripotent stem cells. We therefore describe the use of any culture medium as disclosed in this document for expanding a population of pluripotent stem cells.

We also describe a method for expanding a population of pluripotent stem cells, comprising: (a) providing a population of pluripotent stem cells; (b) providing a culture medium as disclosed in this document; (c) contacting the stem cells with the culture medium; and (d) culturing the stem cells under appropriate conditions.

A method for ‘expanding’ a population of cells is one that involves increasing the number of stem cells in an initial population to generate an expanded population, whilst maintaining pluripotency and without significant differentiation, i.e. one that involves growth and division of stem cells, but not their differentiation.

A variety of substances have been used as extracellular matrix materials for pluripotent stem cell culture, and an appropriate material can readily be selected by a person skilled in the art. An extracellular matrix material may comprise fibronectin, vitronectin, laminin, collagen (particularly collagen II, collagen III or collagen IV), thrombospondin, osteonectin, secreted phosphoprotein I₅ heparan sulphate, dermatan sulphate, gelatine, merosin, tenasin, decorin, entactin or a basement membrane preparation from Engelbreth-Ho Im-S warm (EHS) mouse sarcoma cells (e.g. Matrigel®; Becton Dickenson). A synthetic extracellular matrix material, such as ProNectin (Sigma Z378666) may be used. Mixtures of extracellular matrix materials may be used, if desired.

For example, the extracellular matrix material may comprise fibronectin. Bovine fibronectin, recombinant bovine fibronectin, human fibronectin, recombinant human fibronectin, mouse fibronectin, recombinant mouse fibronectin or synthetic fibronectin may be used.

The extracellular matrix material will normally be coated onto a cell culture vessel, but may (in addition or alternatively) be supplied in solution. A fibronectin solution of about1 mg/ml may be used to coat a cell culture vessel. In some embodiments, a cell culture vessel is coated with fibronectin at about 1 μg/cm² to about 250 μg/cm², or at about 1 μg/cm to about 150 μg/cm . In come embodiments, a cell culture vessel is coated with fibronectin at 8 μg/cm² or 125 μg/cm².

We describe methods comprising culturing the cells in contact with an extracellular matrix material as described elsewhere in this document. For example, we describe a method for expanding a population of pluripotent stem cells, comprising: (a) providing a population of pluripotent stem cells; (b) providing a culture medium as disclosed in this document; (c) contacting the stem cells with the culture medium; and (d) culturing the cells under appropriate conditions and in contact with an extracellular matrix material. We also describe the use of a culture medium as disclosed in this document and an extracellular matrix material to expand a population of pluripotent stem cells.

The methods may comprise a step of passaging stem cells into a culture medium as disclosed in this document. For example, a method for expanding a population of pluripotent stem cells may comprise: (a) providing a population of pluripotent stem cells; (b) providing a culture medium as disclosed in this document; (c) contacting the stem cells with the culture medium; (d) culturing the cells under appropriate conditions; (e) passaging the cells into a culture medium as disclosed in this document; and (f) further culturing the cells under appropriate conditions.

It will be appreciated that the steps of the methods disclosed in this document may be performed in any suitable order or at the same time, as appropriate, and need not be performed in the order in which they are listed. For example, in the above method the step of providing a population of pluripotent stem cells may be performed before, after or at the same time as, the step of providing a culture medium.

Cells may be passaged using known methods, e.g. by incubating the cells with trypsin and EDTA for between 5 seconds and 15 minutes at 37° C. A trypsin substitute (e.g. TrypLE from Invitrogen) may be used, if desired. Collagenase, dispase, accutase or other known reagents may also be used to passage the cells. Passaging is typically required every 2-8 days, such as every 4-7 days, depending on the initial seeding density. In some embodiments, the cell culture methods do not comprise any step of manually selecting undifferentiated cells when the cells are passaged. In some embodiments, the cell culture methods comprise automated passaging of the stem cells, i.e. without manipulation by a laboratory worker.

The pluripotent stem cells will be seeded onto a support at a density that promotes cell proliferation but which limits differentiation. Typically, a plating density of at least 15,000 cells/cm² is used. A plating density of between about 15,000 cells/cm² and about 200,000 cells/cm² may be used. Single-cell suspensions or small cluster of cells will normally be seeded, rather than large clusters of cells, as in known in the art.

The environment used to culture the stem cells may be sterile and temperature stable.

The culture media may be used to expand pluripotent stem cells without the need to adapt the cells to the culture medium, as is commonly required when transferring stem cells into a new culture medium. Various different methods for adapting cell cultures to new media are known in the art. Accordingly, in some embodiments the methods do not include any step of adapting a population of stem cells to a new culture medium, e.g. by gradually changing the components of the medium. We therefore describe a method for expanding a population of pluripotent stem cells, comprising: (a) providing a population of pluripotent stem cells; (b) providing a first culture medium; (c) culturing the cells in the first culture medium under appropriate conditions; (d) providing a second culture medium, which is a culture medium as disclosed in this document, and which is different to the first culture medium; (e) replacing the first culture medium with the second culture medium, exchanging the first culture medium with the second culture medium or passaging the cells from the first culture medium into the second culture medium; and (f) further culturing the cells in the second culture medium under appropriate conditions, wherein the method does not comprise any step of adapting the population of stem cells to the second culture medium.

The methods and uses may involve any culture medium or supplement as described in this document. Accordingly, in some embodiments the methods may be serum and/or serum replacement-free methods. In some embodiments, the methods may be used to culture cells in the absence of contact with a layer of feeder cells.

The cell culture methods may be performed using any suitable cell culture vessel as a support. Cell culture vessels of various shapes and sizes (e.g. flasks, single or multiwell plates, single or multiwell dishes, bottles, jars, vials, bags, bioreactors) and constructed from various different materials (e.g. plastic, glass) are known in the art. A suitable cell culture vessel can readily be selected by a person skilled in the art.

When a cell culture medium described here is used to expand a population of pluripotent stem cells, the total number of undifferentiated, pluripotent stem cells in the population will preferably increase at least 1.5 fold, at least 2 fold, at least 3 fold, at least 4 fold, at least 5 fold, at least 10 fold, at least 20 fold, at least 30 fold, at least 40 fold or at least 50 fold, between the time when a cell culture medium described here is applied to an initial cell population and the end of the culture period.

It will be appreciated that the cells may be passaged one or more times during the culture period, after which the cells may be cultured in different cell culture vessels or cells may be discarded. If cells are cultured in different cell culture vessels after passaging, or if cells are discarded during passaging, this can be taken into account when calculating the fold difference in cell numbers obtained during a known culture period.

A ‘population’ of cells is any number of cells greater than 1 , but is preferably at least 1×10³ cells, at least 1×10⁴ cells, at least 1×10⁵ cells, at least 1×10⁶ cells, at least 1×10⁷ cells, at least 1×10⁸ cells, or at least 1×10⁹ cells.

For example, at least 50%, at least 55%, at least 60%, at least 70%, at least 80%, at least 85%, at least 90%, at least 91%, at least 92%, at least 93%, at least 94% or at least 95%, of the stem cells (% by cell number) in an initial cell population will be undifferentiated, pluripotent and proliferative cells.

In some embodiments, at least 50%, at least 55%, at least 60%, at least 70%, at least 80%, at least 85%, at least 90%, at least 91%, at least 92%, at least 93%, at least 94% or at least 95%, of the stem cells (% by cell number) in an expanded population (z. e. the population after expansion of the initial population using a culture medium or method as disclosed herein) will be undifferentiated, pluripotent and proliferative cells.

Methods for identifying undifferentiated, pluripotent and proliferative stem cells, and for identifying the % of such cells in a population, are known and suitable methods for use with the methods and compositions described here can be selected by a person skilled in the art depending on the stem cell type that is used.

Pluripotent stem cells may be identified by their ability to differentiate into cells of all three germ layers e.g. by determining the ability of the cells to differentiate into cells showing detectable expression of markers specific for all three germ layers. Stem cells can be allowed to form embryoid bodies in vitro, then the embryoid bodies studied to identify cells of all three germ layers. Alternatively, stem cells can be allowed to form teratomas in vivo (e.g. in SCID mice), then the teratomas studied to identify cells of all three germ layers. Accordingly, in preferred embodiments at least 50%, at least 55%, at least 60%, at least 70%, at least 80%, at least 85%, at least 90%, at least 91%, at least 92%, at least 93%, at least 94% or at least 95%, of the stem cells in an expanded population (or in an initial population) are capable of differentiating into cells of all three germ layers in vitro or in vivo.

The genomic integrity of stem cells can be confirmed by karyotype analysis. Stem sells can be karyotyped using known methods. A normal karyotype is where all chromosomes are present (i.e. euploidy) with no noticeable alterations. For example, at least 50%, at least 55%, at least 60%, at least 70%, at least 80%, at least 85%, at least 90%, at least 91%, at least 92%, at least 93%, at least 94% or at least 95%, of the stem cells in an expanded population (or in an initial population) exhibit normal karyotypes.

Pluripotent stem cells may be identified via phenotypic markers. Stem cell markers (both intracellular and extracellular) may be detected using known techniques, such as immunocytochemistry, flow cytometry (e.g. fluorescence-activated cell sorting) and reverse transcription-PCR (RT-PCR). For example, hES cells may be identified via detection of hES cell markers, such as such as OCT-4, stage-specific embryonic antigen 3 (SSEA-3), stage-specific embryonic antigen 4 (SSEA-4), tumour-rejecting antigen 1-60 (TRA-1-60) and tumour-rejecting antigen 1-81 (TRA-1-81). Accordingly, in preferred embodiments at least 50%, at least 55%, at least 60%, at least 70%, at least 80%, at least 85%, at least 90%, at least 91%, at least 92%, at least 93%, at least 94% or at least 95%, of the stem cells in an expanded population (or in an initial population) express OCT-4, SSEA-3, SSEA-4, TRA-1-60 and/or TRA-I -81 at levels appropriate for hES cells.

In some embodiments, at least 50%, at least 55%, at least 60%, at least 70%, at least 80%, at least 85%, at least 90%, at least 91%, at least 92%, at least 93%, at least 94% or at least 95%, of the cells in an expanded population (or in an initial population) will (i) have the ability to differentiate into cells of all three germ layers in vitro or in vivo; (ii) exhibit normal karyotypes; and/or (iii) express the markers OCT-4, SSEA-3, SSEA-4, TRA-1-60 and TRA-1-81 at levels appropriate for hES cells.

Undifferentiated, pluripotent and proliferative stem cells may also be identified by their morphological characteristics. Undifferentiated, pluripotent and proliferative stem cells are readily recognisable by a person skilled in the art. For example, in a normal microscope image hES cells typically have high nuclear/cytoplasmic ratios, prominent nucleoli and compact colony formation with poorly discernable cell junctions.

hES cells may also be identified by determining their alkaline phosphatase activity. hES cells have alkaline phosphatase activity, which can be detected by known methods.

### Culture Medium Supplements

We also describe a culture medium supplement that can be used to produce a culture medium as disclosed in this document.

A “culture medium supplement” is a mixture of ingredients that cannot itself support pluripotent stem cells, but which enables or improves pluripotent stem cell culture when combined with other cell culture ingredients. The supplement can therefore be used to produce a functional cell culture medium described here by combining it with other cell culture ingredients to produce an appropriate medium formulation. The use of culture medium supplements is well known in the art.

We describe a culture medium supplement that comprises a CDK1/2/9 inhibitor and a Bcr-Abl/Src kinase inhibitor. The supplement may contain any CDK1/2/9 inhibitor and Bcr-Abl/Src kinase (or combination of such inhibitors) as described in this document. The supplement may also contain one or more additional cell culture ingredients as disclosed in this document, e.g. one or more cell culture ingredients selected from the group consisting of amino acids, vitamins, inorganic salts, carbon energy sources and buffers.

A culture medium supplement may be a concentrated liquid supplement (e.g. a 2× to 250× concentrated liquid supplement) or may be a dry supplement. Both liquid and dry supplements are well known in the art. A supplement may be lyophilised.

A cell culture medium supplement will typically be sterilized prior to use to prevent contamination, e.g. by ultraviolet light, heating, irradiation or filtration. A culture medium supplement may be frozen (e.g. at −20° C. or 341 80° C.) for storage or transport.

We also describe a hermetically-sealed vessel containing such a culture medium supplement. Hermetically-sealed vessels may be preferred for transport or storage of the culture media supplements disclosed in this document, to prevent contamination. The vessel may be any suitable vessel, such as a flask, a plate, a bottle, a jar, a vial or a bag.

### Absence of Co-culture

The cell culture medium described here is particularly advantageous because it may be used to culture cells without feeder cell contact. The methods described here therefore do not require a layer of feeder cells to support the stem cells.

The culture medium may therefore enable a cell to be cultured in the absence of co-culture.

The term “co-culture” refers to a mixture of two or more different kinds of cells that are grown together. One of the cell types may comprise a feeder cell, for example, stromal feeder cells. The feeder cells may be present in the form of a feeder cell layer.

Thus, in typical pluripotent cell culture, the inner surface of the culture dish is usually coated with a feeder layer of mouse embryonic skin cells that have been treated so they will not divide. The feeder layer provides an adherent surface to enable the pluripotent cells to attach and grow. In addition, the feeder cells release nutrients into the culture medium which are required for pluripotent cell growth.

In the methods and compositions described here, the cell culture medium enables a cell such as a pluripotent cell to be cultured in the absence of such co-culture.

Feeder cell layers are often used to support the culture of pluripotent stem cells, and to inhibit their differentiation. A feeder cell layer is generally a monolayer of cells that is co-cultured with, and which provides a surface suitable for growth of, the pluripotent cells of interest. The feeder cell layer provides an environment in which the cells of interest can grow. Feeder cells are often mitotically inactivated (e.g. by irradiation or treatment with mitomycin C) to prevent their proliferation.

The cell may be cultured as a monolayer or in the absence of feeder cells in the cell culture medium. For example, a pluripotent cell may be cultured in the absence of feeder cells in the cell culture medium.

The pluripotent stem cell may be plated directly onto a culture substrate. The culture substrate may comprise a tissue culture vessel, such as a Petri dish. The vessel may be pre-treated. For example, the cells may be plated onto, and grow on, a gelatinised tissue culture plate.

The cell culture medium enables a cell to be maintained or grown in the absence of co-culture.

The cell culture medium may enable a composition of cultured cells to be feeder cell-free compositions. A composition is conventionally considered to be feeder cell-free if the pluripotent stem cells in the composition have been cultured for at least one passage in the absence of a feeder cell layer.

A feeder cell-free composition will normally contain less than about 5%, less than about 4%, less than about 3%, less than about 2%, or less than about 1% feeder cells (expressed as a % of the total number of cells in the composition).

Conversely, a feeder cell-free composition will normally contain more than about 95%, more than about 96%, more than about 97%, more than about 98%, or more than about 99% pluripotent cells (expressed as a % of the total number of cells in the composition).

### Naïve Pluripotent Cells

A cell cultured in the cell culture medium described here may display one or more characteristics of a pluripotent cell, such as a naïve pluripotent cell.

Such characteristics may include expression of a pluripotent cell marker, for example a naïve pluripotent cell marker. The naïve pluripotent cell marker may comprise a naïve-specific transcription factor.

The naïve pluripotent cell marker may comprise any one or more of the following: CD130 (Gene ID: 3572), CD75 (Gene ID: 6480), DNMT3L (Gene ID: 29947), DPPA5 (Gene ID: 340168), KLF5 (Gene ID: 688), TFCP2L1 (Gene ID: 29842), KLF4 (Gene ID: 9314), DPPA3 (Gene ID: 359787), NANOG (Gene ID: 79923), KLF17 (Gene ID: 128209), POU5F1 (Gene ID: 5460) or PRDM14 (Gene ID: 63978).

A cell cultured in the cell culture medium may display nuclear-specific localization of TFE3. It may preferentially utilize the distal POU5F1 enhancer.

The cell culture medium may therefore be used to reprogram primed pluripotent stem cells to naïve pluripotent stem cells.

### Feeder-independent Naïve ESCs (FINE)

We disclose a method of producing a feeder-independent culture of naïve embryonic stem cells.

We also disclose cells converted using such a process. Such converted cells may conveniently be termed FINE cells (feeder-independent naïve ESCs).

Using the methods and compositions described here, it is possible to convert a primed pluripotent stem cell into a naïve pluripotent stem cell.

The method comprises culturing a pluripotent cell in the cell culture medium described here. The cell may comprise a primed pluripotent stem cell, such as a primed embryonic stem cell. The cell may be cultured in the cell culture medium for a number of generations to promote the expression of naïve stem cell characteristics.

The cell may be cultured in FINE media, which consists of basal media (1:1 ratio of F12 DMEM and Neurobasal media, 1× N2 supplement and 1× B2 supplement, 1× L-Glutamine, 1× Non-essential amino acids, 0.1 mM of B-mercaptoethanol and 62.5 ng/ml of BSA) supplemented with 0.1 μM of Dasatinib, 0.1 μM AZD5438, 0.1 μLM SB590885, 1 μM of PD0325901, 10 04 of Y-27632, 20 ng/ml of human recombinant LIF , 20 ng/ml of Activin A and 8 ng/ml of bFGF.

The conversion may comprise culturing cells in FINE media under normoxia conditions, for example between 3-6 days, such as between 4-5 days, such as 4 days, 4.5 days or 5 days.

The conversion may comprise culturing cells in FINE media under a combination of normoxia and hypoxia. For example, the cells may be cultured under normoxia for a single passage (P0) followed by further passages, such as 4, 5, 6 or more passages, such as 5 further passages (denoted P1-P5) under hypoxia. The P0 culture under normoxia may be on any suitable substrate such as Matrigel. The further passages may be on reduced growth factor Matrigel.

Under these conditions, the expression of pluripotency markers such as POU5F1 and PRDM14 may be transiently downregulated. Such expression may return to normal levels by for example the 5^{th} passage.

The culture medium may be exchanged or replenished as needed, for example daily.

At the end of the conversion process, the feeder-independent naïve cells (FINE cells) may be further cultured. For example, the cells may be passaged as single cells.

The cells may be passaged further, for example at a 1:2, 1:3 or 1:4 ratio.

The cells may be cultured in FINE media for at least 3 passages, at least 4 passages or at least 5 passages.

The FINE cells converted by this process are capable of expressing one or more characteristics of naïve pluripotent cells, as described elsewhere in this document. For example, naïve-specific transcription factors KLF4, KLF17 and TFE3 may be localized in the cell nucleus in converted FINE cells.

The FINE cells may express of stage-specific ERVs, such as LTR7Y and HERVH, at higher levels than cells not exposed to conversion, such as the starting cells, such as primed pluripotent stem cells.

The FINE cells may exhibit heterogeneous expression of naïve transcription factors, such as NANOG, KLF4 and KLF17. The FINE cells may exhibit homogeneous expression of naïve surface markers, such as CD75 and CD130.

### Media and Feeder Cells

Media for isolating and propagating pluripotent stem cells can have any of several different formulas, as long as the cells obtained have the desired characteristics, and can be propagated further.

Cell culture media typically contain a large number of ingredients, which are necessary to support maintenance of the cultured cells. The cell culture medium will therefore normally contain many other ingredients in addition to a CDK1/2/9 inhibitor and a Bcr-Abl/Src kinase inhibitor.

Suitable combinations of ingredients may be readily be formulated by a person skilled in the art.

The cell culture medium will generally be a nutrient solution comprising standard cell culture ingredients, such as amino acids, vitamins, inorganic salts, a carbon energy source and a buffer.

The cell culture medium may be generated by modification of an existing cell culture medium. A person skilled in the art understands the types of culture media that might be used for pluripotent stem cell culture.

Potentially suitable cell culture media are available commercially, and include Dulbecco's Modified Eagle Media (DMEM), Minimal Essential Medium (MEM), Knockout-DMEM (KO-DMEM), Glasgow Minimal Essential Medium (G-MEM), Basal Medium Eagle (BME), DMEM/Ham's F 12, Advanced DMEM/Ham's F 12, Iscove's Modified Dulbecco's Media and Minimal Essential Media (MEM).

The cell culture medium may comprise one or more amino acids. A person skilled in the art would understand the appropriate types and amounts of amino acids for use in stem cell culture media. Amino acids which may be present include L-alanine, L-arginine, L-asparagine, L-aspartic acid, L-cysteine, L-cystine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, L-valine and combinations thereof. Some culture media will contain all of these amino acids. Generally, each amino acid when present is present at about 0.001 to about 1 g/L of medium (usually at about 0.01 to about 0.15 g/L), except for L-glutamine which is present at about 0.05 to about 1 g/L (usually about 0.1 to about 0.75 g/L). The amino acids may be of natural or synthetic origin.

The cell culture medium may comprise one or more vitamins. A person skilled in the art would understand the appropriate types and amounts of vitamins for use in stem cell culture media. Vitamins which may be present include thiamine (vitamin B1 ), riboflavin (vitamin B2), niacin (vitamin B3), D-calcium pantothenate (vitamin B5), pyridoxal/pyridoxamine/pyridoxine (vitamin B6), folic acid (vitamin B9), cyanocobalamin (vitamin B 12), ascorbic acid (vitamin C), calciferol (vitamin D2), DL-alpha tocopherol (vitamin E), biotin (vitamin H) and menadione (vitamin K).

The cell culture medium may comprise one or more inorganic salts. A person skilled in the art would understand the appropriate types and amounts of inorganic salts for use in stem cell culture media. Inorganic salts are typically included in culture media to aid maintenance of the osmotic balance of the cells and to help regulate membrane potential. Inorganic salts which may be present include salts of calcium, copper, iron, magnesium, potassium, sodium, zinc. The salts are normally used in the form of chlorides, phosphates, sulphates, nitrates and bicarbonates. Specific salts that may be used include CaCl₂, CuSO₄—5H₂P, Fe(NO₃)·9H₂O, FeSO₄·7H₂O, MgCl₂, MgSO₄, KCl, NaHCO₃, NaCl, Na₂HPO₄, Na₂HPO₄·H₂O and ZnSO₄7H₂O.

The osmolarity of the medium may be in the range from about 200 to about 400 mθsm/kg, in the range from about 290 to about 350 mθsm/kg, or in the range from about 280 to about 310 mθsm/kg. The osmolarity of the medium may be less than about 300 mθsm/kg (e.g. about 280 mθsm/kg).

The cell culture medium may comprise a carbon energy source, in the form of one or more sugars. A person skilled in the art would understand the appropriate types and amounts of sugars to use in stem cell culture media. Sugars which may be present include glucose, galactose, maltose and fructose. The sugar may comprise glucose, particularly D-glucose (dextrose). A carbon energy source will normally be present at between about 1 and about 10 g/L.

The cell culture medium may comprise a buffer. A suitable buffer can readily be selected by a person skilled in the art. The buffer may be capable of maintaining the pH of the culture medium in the range about 6.5 to about 7.5 during normal culturing conditions, such as around pH 7.0. Buffers that may be used include carbonates (e.g. NaHCO₃), chlorides (e.g. CaCl₂), sulphates (e.g. MgSO₄) and phosphates (e.g. NaH₂PO₄). These buffers are generally used at about 50 to about 500 mg/l. Other buffers such as N-[2-hydroxyethyl]-piperazine-N′-[2-ethanesul-phonic acid] (HEPES) and 3-[N-morpholinoj-propanesulfonic acid (MOPS) may also be used, normally at around 1000 to around 10,000 mg/l.

The cell culture medium may contain serum. Serum obtained from any appropriate source may be used, including foetal bovine serum (FBS), goat serum or human serum. For example, human serum is used. Serum may be used at between about 1% and about 30% by volume of the medium, according to conventional techniques.

Alternatively or in addition, the cell culture medium may contain a serum replacement. Various different serum replacement formulations are commercially available and are known to a person skilled in the art. Where a serum replacement is used, it may be used at between about 1% and about 30% by volume of the medium, according to conventional techniques.

The cell culture medium may be serum-free and/or serum replacement-free. A serum-free medium is one that contains no animal serum of any type. Serum-free media may be preferred to avoid possible xeno-contamination of the stem cells. A serum replacement-free medium is one that has not been supplemented with any commercial serum replacement formulation.

The culture medium may comprise cholesterol or a cholesterol substitute. Cholesterol may be provided in the form of the HDL or LDL extract of serum. Where the HDL or LDL extract of serum is used, the extract of human serum may be employed. The optimal amount of cholesterol or cholesterol substitute can readily be determined from the literature or by routine experimentation. A synthetic cholesterol substitute may be used rather than cholesterol derived from an animal source. For example, Synthecol™ (Sigma S5442) may be used in accordance with the manufacturer's instructions.

The culture medium may further comprise transferrin or a transferrin substitute. Transferrin may be provided in the form of recombinant transferrin or in the form of an extract from serum. Recombinant human transferrin or an extract of human serum may be used. An iron chelate compound may be used as a transferrin substitute. Suitable iron chelate compounds are known to a person skilled in the art, and include ferric citrate chelates and ferric sulphate chelates. The optimal amount of transferrin or transferrin substitute can readily be determined from the literature or by routine experimentation. In some embodiments, The cell culture medium may comprise transferrin at about 5.5 μg/ml.

The culture medium may further comprise albumin or an albumin substitute, such as bovine serum albumin (BSA), human serum albumin (HSA), a plant hydrolysate (e.g. a rice or soy hydrolysate), Albumax® I or Albumax® II. The optimal amount of albumin or albumin substitute can readily be determined from the literature or by routine experimentation. In some embodiments, The cell culture medium may comprise albumin at about 0.5 μg/ml.

The culture medium may further comprise insulin or an insulin substitute. Natural or recombinant insulin may be used. A zinc-containing compound may be used as an insulin substitute, e.g. zinc chloride, zinc nitrate, zinc bromide or zinc sulphate. The optimal amount of insulin or insulin substitute can readily be determined from the literature or by routine experimentation. In some embodiments, The cell culture medium may comprise insulin at about 10 μg/ml.

The culture medium may comprise progesterone, putrescine, and/or selenite. If selenite is present, it may be in the form of sodium selenite. The optimal amount of these ingredients can readily be determined from the literature or by routine experimentation.

The cell culture medium may comprise one or more additional nutrients or growth factors that have previously been reported to benefit pluripotent stem cell culture.

For example, a culture medium may comprise fibroblast growth factor (FGF), transforming growth factor beta 1 (TGFp1), leukaemia inhibitor factor (LIF), ciliary neurotrophic factor (CNTF), interleukin 6 (IL-6) or stem cell factor (SCF). Antibodies or other ligands that bind to the receptors for such substances may also be used. Any form of FGF suitable for pluripotent stem cell culture may be used, e.g. basic FGF (bFGF; FGF-2), FGF-4, or homologs or analogues thereof. In some embodiments, bFGF is used. bFGF may be used at from about 1 ng/ml to about 50 ug/ml, e.g. at about 5 ng/ml, at about 10 ng/ml, or at about 40 ng/ml.

The cell culture medium may comprise one or more trace elements, such as ions of barium, cobalt, iodine, manganese, chromium, copper, nickel, selenium, vanadium, titanium, germanium, molybdenum, silicon, iron, fluorine, silver, rubidium, tin, zirconium, cadmium, zinc and/or aluminium.

A culture medium may further comprise phenol red as a pH indicator, to enable the status of the medium to be easily monitored (e.g. at about 5 to about 50 mg/litre).

The medium may comprise a reducing agent, such as β-mercaptoethanol at a concentration of about 0.1 mM.

‘N2 Supplement’ (available from Invitrogen, Carlsbad, Calif.; catalogue no. 17502-048; and from PAA Laboratories GmbH, Pasching, Austria; www.paa.com; catalogue no. F005-004; Bottenstein & Sato, PNAS, 76(1):514-517, 1979) may be used to formulate a culture medium that comprises contains transferrin, insulin, progesterone, putrescine, and sodium selenite. N2 Supplement is supplied by PAA Laboratories GmbH as a 100×liquid concentrate, containing 500 μg/ml human transferrin, 500 μg/ml bovine insulin, 0.63 μg/ml progesterone, 1611 μg/ml putrescine, and 0.52 μg/ml sodium selenite. N2 Supplement may be added to a culture medium as a concentrate or diluted before addition to a culture medium. It may be used at a I× final concentration or at other final concentrations. Use of N2 Supplement is a convenient way to incorporate transferrin, insulin, progesterone, putrescine and sodium selenite into the cell culture medium.

‘B27 Supplement’ (available from Invitrogen, Carlsbad, Calif.; www.invitrogen.com; currently catalogue no. 17504-044; and from PAA Laboratories GmbH, Pasching, Austria; www.paa.com; catalogue no. F01-002; Brewer et al, J Neurosci Res., 35(5):567-76, 1993) may be used to formulate a culture medium that comprises biotin, cholesterol, linoleic acid, linolenic acid, progesterone, putrescine, retinol, retinyl acetate, sodium selenite, triiodothyronine (T3), DL-alpha tocopherol (vitamin E), albumin, insulin and transferrin. B27 Supplement is supplied by PAA Laboratories GmbH as a liquid 50× concentrate, containing amongst other ingredients biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin. Of these ingredients at least linolenic acid, retinol, retinyl acetate and tri-iodothyronine (T3) are nuclear hormone receptor agonists as described elsewhere in this document. B27 Supplement may be added to a culture medium as a concentrate or diluted before addition to a culture medium. It may be used at a Ix final concentration or at other final concentrations. Use of B27 Supplement is a convenient way to incorporate biotin, cholesterol, linoleic acid, linolenic acid, progesterone, putrescine, retinol, retinyl acetate, sodium selenite, tri-iodothyronine (T3), DL-alpha tocopherol (vitamin E), albumin, insulin and transferrin into the cell culture medium.

The cell culture medium will normally be formulated in deionized, distilled water. The cell culture medium will typically be sterilized prior to use to prevent contamination, e.g. by ultraviolet light, heating, irradiation or filtration. The culture medium may be frozen (e.g. at −200 C or −800 C) for storage or transport. The medium may contain one or more antibiotics to prevent contamination. The medium may have an endotoxin content of less that 0.1 endotoxin units per ml, or may have an endotoxin content less than 0.05 endotoxin units per ml. Methods for determining the endotoxin content of culture media are known in the art.

### Stem Cells

As used in this document, the term “stem cell” refers to a cell that on division faces two developmental options: the daughter cells can be identical to the original cell (self-renewal) or they may be the progenitors of more specialised cell types (differentiation).

The stem cell is therefore capable of adopting one or other pathway (a further pathway exists in which one of each cell type can be formed). Stem cells are therefore cells which are not terminally differentiated and are able to produce cells of other types.

Stem cells as referred to in this document may include totipotent stem cells, pluripotent stem cells, and multipotent stem cells.

Totipotent Stem Cells

The term “totipotent” cell refers to a cell which has the potential to become any cell type in the adult body, or any cell of the extraembryonic membranes (e.g., placenta). Thus, the only totipotent cells are the fertilized egg and the first 4 or so cells produced by its cleavage.

Pluripotent Stem Cells

“Pluripotent stem cells” are true stem cells, with the potential to make any differentiated cell in the body. However, they cannot contribute to making the extraembryonic membranes which are derived from the trophoblast. Several types of pluripotent stem cells have been found.

Embryonic Stem Cells

Embryonic Stem (ES) cells may be isolated from the inner cell mass (ICM) of the blastocyst, which is the stage of embryonic development when implantation occurs.

Embryonic Germ Cells

Embryonic Germ (EG) cells may be isolated from the precursor to the gonads in aborted fetuses.

Embryonic Carcinoma Cells

Embryonic Carcinoma (EC) cells may be isolated from teratocarcinomas, a tumor that occasionally occurs in a gonad of a fetus. Unlike the first two, they are usually aneuploid. All three of these types of pluripotent stem cells can only be isolated from embryonic or fetal tissue and can be grown in culture. Methods are known in the art which prevent these pluripotent cells from differentiating.

Adult Stem Cells

Adult stem cells comprise a wide variety of types including neuronal, skin and the blood forming stem cells which are the active component in bone marrow transplantation. These latter stem cell types are also the principal feature of umbilical cord-derived stem cells. Adult stem cells can mature both in the laboratory and in the body into functional, more specialised cell types although the exact number of cell types is limited by the type of stem cell chosen.

Multipotent Stem Cells

Multipotent stem cells are true stem cells but can only differentiate into a limited number of types. For example, the bone marrow contains multipotent stem cells that give rise to all the cells of the blood but not to other types of cells. Multipotent stem cells are found in adult animals. It is thought that every organ in the body (brain, liver) contains them where they can replace dead or damaged cells.

Methods of characterising stem cells are known in the art, and include the use of standard assay methods such as clonal assay, flow cytometry, long-term culture and molecular biological techniques e.g. PCR, RT-PCR and Southern blotting.

In addition to morphological differences, human and murine pluripotent stem cells differ in their expression of a number of cell surface antigens (stem cell markers). Antibodies for the identification of stem cell markers including the Stage-Specific Embryonic Antigens 1 and 4 (SSEA-1 and SSEA-4) and Tumor Rejection Antigen 1-60 and 1-81 (TRA-1-60, TRA-1-81) may be obtained commercially, for example from Chemicon International, Inc (Temecula, CA, USA).

The immunological detection of these antigens using monoclonal antibodies has been widely used to characterize pluripotent stem cells (Shamblott M. J. et. al. (1998) PNAS 95: 13726-13731; Schuldiner M. et. al. (2000). PNAS 97: 11307-11312; Thomson J. A. et. al. (1998). Science 282: 1145-1147; Reubinoff B. E. et. al. (2000). Nature Biotechnology 18: 399-404; Henderson J. K. et. al. (2002). Stem Cells 20: 329-337; Pera M. et. al. (2000). J. Cell Science 113: 5-10.).

### Sources of Stem Cells

Stem cells of various types, which may include the following non-limiting examples, may be used in the methods and compositions described here for producing progenitor cells, progenitor cell lines and differentiated cells.

U.S. Pat. No. 5,851,832 reports multipotent neural stem cells obtained from brain tissue. U.S. Pat. No. 5,766,948 reports producing neuroblasts from newborn cerebral hemispheres. U.S. Pat. Nos. 5,654,183 and 5,849,553 report the use of mammalian neural crest stem cells. U.S. Pat. No. 6,040,180 reports in vitro generation of differentiated neurons from cultures of mammalian multipotential CNS stem cells. WO 98/50526 and WO 99/01159 report generation and isolation of neuroepithelial stem cells, oligodendrocyte-astrocyte precursors, and lineage-restricted neuronal precursors. U.S. Pat. No. 5,968,829 reports neural stem cells obtained from embryonic forebrain and cultured with a medium comprising glucose, transferrin, insulin, selenium, progesterone, and several other growth factors.

Primary liver cell cultures can be obtained from human biopsy or surgically excised tissue by perfusion with an appropriate combination of collagenase and hyaluronidase. Alternatively, EP 0 953 633 A1 reports isolating liver cells by preparing minced human liver tissue, resuspending concentrated tissue cells in a growth medium and expanding the cells in culture. The growth medium comprises glucose, insulin, transferrin, T3, FCS, and various tissue extracts that allow the hepatocytes to grow without malignant transformation. The cells in the liver are thought to contain specialized cells including liver parenchymal cells, Kupffer cells, sinusoidal endothelium, and bile duct epithelium, and also precursor cells (referred to as “hepatoblasts” or “oval cells”) that have the capacity to differentiate into both mature hepatocytes or biliary epithelial cells (L. E. Rogler, Am. J. Pathol. 150:591, 1997; M. Alison, Current Opin. Cell Biol. 10:710, 1998; Lazaro et al., Cancer Res. 58:514, 1998).

U.S. Pat. No. 5,192,553 reports methods for isolating human neonatal or fetal hematopoietic stem or progenitor cells. U.S. Pat. No. 5,716,827 reports human hematopoietic cells that are Thy-1 positive progenitors, and appropriate growth media to regenerate them in vitro. U.S. Pat. No. 5,635,387 reports a method and device for culturing human hematopoietic cells and their precursors. U.S. Pat. No. 6,015,554 describes a method of reconstituting human lymphoid and dendritic cells.

U.S. Pat. No. 5,486,359 reports homogeneous populations of human mesenchymal stem cells that can differentiate into cells of more than one connective tissue type, such as bone, cartilage, tendon, ligament, and dermis. They are obtained from bone marrow or periosteum. Also reported are culture conditions used to expand mesenchymal stem cells. WO 99/01145 reports human mesenchymal stem cells isolated from peripheral blood of individuals treated with growth factors such as G-CSF or GM-CSF. WO 00/53795 reports adipose-derived stem cells and lattices, substantially free of adipocytes and red cells. These cells reportedly can be expanded and cultured to produce hormones and conditioned culture media.

Stem cells of any vertebrate species can be used. Included are stem cells from humans; as well as non-human primates, domestic animals, livestock, and other non-human mammals

Amongst the stem cells suitable for use in methods and compositions described here are primate pluripotent stem (pPS) cells derived from tissue formed after gestation, such as a blastocyst, or fetal or embryonic tissue taken any time during gestation. Non-limiting examples are primary cultures or established lines of embryonic stem cells.

### Media and Feeder Cells

Media for isolating and propagating pPS cells can have any of several different formulas, as long as the cells obtained have the desired characteristics, and can be propagated further. Suitable sources are as follows: Dulbecco's modified Eagles medium (DMEM), Gibco#11965-092; Knockout Dulbecco's modified Eagles medium (KO DMEM), Gibco#10829-018; 200 mM L-glutamine, Gibco#15039-027; non-essential amino acid solution, Gibco 11140-050; beta-mercaptoethanol, Sigma#M7522; human recombinant basic fibroblast growth factor (bFGF), Gibco#13256-029. Exemplary serum-containing embryonic stem (ES) medium is made with 80% DMEM (typically KO DMEM), 20% defined fetal bovine serum (FBS) not heat inactivated, 0.1 mM non-essential amino acids, 1 mM L-glutamine, and 0.1 mM beta-mercaptoethanol. The medium is filtered and stored at 4 degrees C. for no longer than 2 weeks. Serum-free embryonic stem (ES) medium is made with 80% KO DMEM, 20% serum replacement, 0.1 mM non-essential amino acids, 1 mM L-glutamine, and 0.1 mM beta-mercaptoethanol. An effective serum replacement is Gibco#10828-028. The medium is filtered and stored at 4 degrees C. for no longer than 2 weeks. Just before use, human bFGF is added to a final concentration of 4 ng/mL (Bodnar et al., Geron Corp, International Patent Publication WO 99/20741).

Feeder cells (where used) are propagated in mEF medium, containing 90% DMEM (Gibco#11965-092), 10% FBS (Hyclone#30071-03), and 2 mM glutamine. mEFs are propagated in T150 flasks (Coming#430825), splitting the cells 1:2 every other day with trypsin, keeping the cells sub confluent. To prepare the feeder cell layer, cells are irradiated at a dose to inhibit proliferation but permit synthesis of important factors that support human embryonic stem cells (.about.4000 rads gamma irradiation). Six-well culture plates (such as Falcon#304) are coated by incubation at 37 degrees C. with 1 mL 0.5% gelatin per well overnight, and plated with 375,000 irradiated mEFs per well. Feeder cell layers are typically used 5 h to 4 days after plating. The medium is replaced with fresh human embryonic stem (hES) medium just before seeding pPS cells.

Conditions for culturing other stem cells are known, and can be optimized appropriately according to the cell type. Media and culture techniques for particular cell types referred to in the previous section are provided in the references cited.

### Embryonic Stem Cells

Embryonic stem cells can be isolated from blastocysts of members of the primate species (Thomson et al., Proc. Natl. Acad. Sci. USA 92:7844, 1995). Human embryonic stem (hES) cells can be prepared from human blastocyst cells using the techniques described by Thomson et al. (U.S. Pat. No. 5,843,780; Science 282:1145, 1998; Curr. Top. Dev. Biol. 38:133 ff., 1998) and Reubinoff et al, Nature Biotech. 18:399,2000.

Briefly, human blastocysts are obtained from human in vivo preimplantation embryos. Alternatively, in vitro fertilized (IVF) embryos can be used, or one cell human embryos can be expanded to the blastocyst stage (Bongso et al., Hum Reprod 4: 706, 1989). Human embryos are cultured to the blastocyst stage in G1.2 and G2.2 medium (Gardner et al., Fertil. Steril. 69:84, 1998). Blastocysts that develop are selected for embryonic stem cell isolation. The zona pellucida is removed from blastocysts by brief exposure to pronase (Sigma). The inner cell masses are isolated by immunosurgery, in which blastocysts are exposed to a 1:50 dilution of rabbit anti-human spleen cell antiserum for 30 minutes, then washed for 5 minutes three times in DMEM, and exposed to a 1:5 dilution of Guinea pig complement (Gibco) for 3 minutes (see Solter et al., Proc. Natl. Acad. Sci. USA 72:5099, 1975). After two further washes in DMEM, lysed trophectoderm cells are removed from the intact inner cell mass (ICM) by gentle pipetting, and the ICM plated on mEF feeder layers.

After 9 to 15 days, inner cell mass-derived outgrowths are dissociated into clumps either by exposure to calcium and magnesium-free phosphate-buffered saline (PBS) with 1 mM EDTA, by exposure to dispase or trypsin, or by mechanical dissociation with a micropipette; and then replated on mEF in fresh medium. Dissociated cells are replated on mEF feeder layers in fresh embryonic stem (ES) medium, and observed for colony formation. Colonies demonstrating undifferentiated morphology are individually selected by micropipette, mechanically dissociated into clumps, and replated. embryonic stem cell-like morphology is characterized as compact colonies with apparently high nucleus to cytoplasm ratio and prominent nucleoli. Resulting embryonic stem cells are then routinely split every 1-2 weeks by brief trypsinization, exposure to Dulbecco's PBS (without calcium or magnesium and with 2 mM EDTA), exposure to type IV collagenase (.about.200 U/mL; Gibco) or by selection of individual colonies by micropipette. Clump sizes of about 50 to 100 cells are optimal.

Commercially available hES cell lines may also be used.

### Further Aspects

Further aspects and embodiments of the invention are now set out in the following numbered Paragraphs; it is to be understood that the invention encompasses these aspects:

Paragraph 1. A method of culturing a cell in the presence of: (a) a CDK1/2/9 inhibitor such as AZD5438 (4-[2-Methyl-1-(1-methylethyl)-1H-imidazol-5-yl]-N-[4-(methylsulfonyl)phenyl]-2-pyrimidinamine, AZD); and (b) a Bcr-Abl/Src kinase inhibitor such as Dasatinib (N-(2-chloro-6-methylphenyl)-2-[[6-[4-(2-hydroxyethyl)-1-piperazinyl]-2-methyl-4-pyrimidinyl]amino]-5-thiazole carboxamide monohydrate, DASA); in which the method is capable of maintaining or increasing the expression of a naïve pluripotent stem cell marker in the cell.

Paragraph 2. A method according to Paragraph 1, in which the method does not include co-culture with feeder cells, and in which the expression of a naïve pluripotent stem cell marker is increased compared to culture in the absence of each of (a), (b) and feeder cells.

Paragraph 3. A method according to Paragraph 1 or 2, in which the method comprises culturing the cell for 5 or more passages.

Paragraph 4. A method according to Paragraph 1, 2 or 3, in which the naïve pluripotent stem cell marker comprises CD130 (Gene ID: 3572), CD75 (Gene ID: 6480), DNMT3L (Gene ID: 29947), DPPA5 (Gene ID: 340168), KLF5 (Gene ID: 688), TFCP2L1 (Gene ID: 29842), KLF4 (Gene ID: 9314), DPPA3 (Gene ID: 359787), NANOG (Gene ID: 79923), KLF17 (Gene ID: 128209), POU5F1 (Gene ID: 5460) or PRDM14 (Gene ID: 63978). (gene and protein name)

Paragraph 5. A method according to any preceding Paragraph, in which the method is capable of decreasing the expression of a primed pluripotent stem cell marker such as ZIC2 (Gene ID: 7546) and B3GAT1 (Gene ID: 27087).

Paragraph 6. A method according to any preceding Paragraph, in which the cell culture medium comprises AZD5438 at a concentration of 0.1 μM or more, such as 0.1 μM to 0.5 μM and Dasatinib at a concentration of 0.1 μM or more, such as 0.1 μM to 0.404, preferably AZD5438 at 0.2 μM and Dasatinib at 0.2 μM.

Paragraph 7. A method according to any preceding Paragraph, in which the cell comprises a naïve pluripotent stem cell, preferably a mammalian naïve pluripotent stem cell, such as a human naïve pluripotent stem cell.

Paragraph 8. A method according to Paragraph 7, in which the method is capable of: (a) maintaining the naïve pluripotent stem cell in a naïve state; and/or (b) maintaining the survival of a naïve pluripotent stem cell preferably after at least 5 passages, preferably after at least 8 passages.

Paragraph 9. A method according to any preceding Paragraph, in which the cell comprises a primed pluripotent stem cell, preferably a mammalian primed pluripotent stem cell, such as a human primed pluripotent stem cell, in which the method re-programs the primed pluripotent stem cell into a naïve pluripotent stem cell.

Paragraph 10. A method according to any of Paragraphs 1 to 6, in which the cell comprises a somatic cell, preferably a mammalian somatic cell, such as a human somatic cell, in which the method re-programs the somatic cell into a naïve pluripotent stem cell, and in which the method preferably further comprises up-regulating the expression of Oct4 (Pou5f1), Sox2, Klf4 and c-Myc in the somatic cell.

Paragraph 11. A method according to any preceding Paragraph, in which the method comprises culturing the cell in the further presence of any one or more of SB590885 ((NE)-N-[5-[2-[4-[2-(dimethylamino) ethoxy]phenyl]-5-pyridin-4-yl-1H-imidazol-4-yl]-2,3-dihydroinden-1-ylidene]hydroxylamine), PD0325901 (N-[(2R)-2,3-dihydroxypropoxy]-3,4-difluoro-2-(2-fluoro-4-iodoanilino)benzamide) and Y-27632 (4-[(1R)-1-aminoethyl]-N-pyridin-4-ylcyclohexane-1-carboxamide), such as one or more of 0.5 μM SB590885, 1 μM of PD0325901 and 10 04 of Y-27632.

Paragraph 12. A method according to any preceding Paragraph, in which the method comprises culturing the cell in the further presence of any one or more of LIF (UniProtKB-P15018), Activin A (UniProtKB-P08476), and bFGF (UniProtKB-P09038), such as one or more of 20 ng/ml of human recombinant LIF, 20 ng/ml of Activin A and 8 ng/ml of bFGF.

Paragraph 13. A cell culture medium comprising a CDK1/2/9 inhibitor such as AZD5438 (AZD) such as at a concentration of 0.1 μM or more, such as 0.1 μM to 0.5 μM, preferably 0.2 μM and a Bcr-Abl/Src kinase inhibitor such as Dasatinib (DASA) such as at a concentration of 0.1 μM or more, such as 0.1 μM to 0.504, preferably 0.2 μM .

Paragraph 14. A cell culture medium according to Paragraph 13, in which the cell culture medium is capable of maintaining or increasing the expression of a naïve pluripotent stem cell marker in a cell in the absence of co-culture.

Paragraph 15. A cell culture medium according to Paragraph 13 or 14, in which the cell culture medium further comprises one or more of SB590885, PD0325901, Y-27632, LIF, Activin A and bFGF, such as one or more of 0.5 μM SB590885, 1 μM of PD0325901, 10 04 of Y-27632, 20 ng/ml of human recombinant LIF, 20 ng/ml of Activin A and 8 ng/ml of bFGF.

Paragraph 16. A cell culture medium according to Paragraph 13, 14 or 15, in which the cell culture medium comprises basal media comprising 1:1 ratio of F12 DMEM (STEMCELL

Technologies) and Neurobasal media (Gibco), 1× N2 supplement (Gibco) and 1× B2 supplement (Gibco), 1× L-Glutamine (Gibco), 1× Non-essential amino acids (Gibco), 0.1 mM of B-mercaptoethanol (Sigma) and 62.5 ng/ml of bovine serum albumin (BSA, Sigma).

Paragraph 17. A cell culture medium according to any of Paragraphs 13 to 16, in which the cell culture medium comprises the components shown in Table El, Table E2 and Table E3 at the concentrations set out in the tables.

Paragraph 18. A method of propagation of a naïve pluripotent stem cell, the method comprising culturing the naïve pluripotent stem cell in a cell culture medium according to any of Paragraphs 13 to 17.

Paragraph 19. A method of re-programming a primed pluripotent stem cell into a naïve pluripotent stem cell, the method comprising culturing the primed pluripotent stem cell in a cell culture medium according to any of Paragraphs 13 to 17.

### EXAMPLES

### Example 1. Materials and Methods—Experimental Procedures

Cell Lines

H1 (WA-01, passage 23-40) line was used for all experiments unless specified otherwise. Other lines used are HES3 (ES-03, passage 79-90), H9 (WA-09, passage 35) and iPSCs (GM23338, passage 35) cells.

Primed mTeSR1 Culture

hESCs were propagated in mTeSR1 (STEMCELL Technologies). Cells were cultured on 30× diluted Matrigel matrix (Corning) coated dishes under normoxia (37° C., 21% 02, 5% CO₂). Cell culture plates were coated with Matrigel for at least 1 hr in the incubator before use. Culture medium was refreshed daily. Cells were subcultured using 1 mg/ml Dispase in DMEM/F12 (STEMCELL Technologies) every 3-6 days according to manufacturer's protocol.

Naïve 3 iL Culture

3iL cultured cells were propagated as previously described (Chan et al., 2013).

Naïve 4 iLA +Feeder Culture

4 iLA+feeder cells were cultured as previously described (Theunissen et al., 2016). Cells were cultured in hypoxia conditions (5% O₂, 5% CO₂). Medium was refreshed daily. Cells were subcultured using TrypLE (Life Technologies) every 4-7days.

Conversion of the Primed hESC to Naïve hESC with Fine Culture

Primed cells were seeded on 30× diluted Matrigel at a passage ratio of 1:6. Cells were seeded in clumps and keep in mTeSR1 culture for 48 hr. For conversion to naïve cell state, we removed mTeSR1 media and added FINE culture media, which consists of a basal media (1:1 ratio of F12 DMEM and Neurobasal (Gibco) media, 1× N2 supplement (Gibco) and 1× B2 supplement (Gibco), 1× L-Glutamine (Gibco), 1× Non-essential amino acids (Gibco), 0.1 mM of B-mercaptoethanol (Sigma) and 62.5 ng/ml of BSA (Sigma)) supplemented with 0.1 μM of Dasatinib (Selleckchem), 0.1 μM AZD5438 (TOCRIS), 0.1 04 SB590885 (Sigma), 1 μM of PD0325901 (Sigma), 10 μM of Y-27632 (STEMCELL Technologies), 20 ng/ml of human recombinant LIF (Peprotech), 20 ng/ml of Activin A (STEMCELL Technologies) and 8 ng/ml of bFGF (Gibco). Cells are incubated at normoxia conditions (21% O₂, 5% CO₂) for 4-5 days. FINE culture media is replenished daily. At the end of conversion, cells are passaged as single cells using TrypLE (Gibco) solution on Reduce Growth Factor Matrigel (Corning) coated plates (dishes are coated for at least 1 hr before use). Briefly, cells are washed with 1× PBS and 500 μl of TrypLE is added to each 3.5 cm well (6 well plate, Falcon) of hESCs. Cells were incubated at 37° C. for 1-2 mins. When cells start to detach from each other and remains adherent to the plate, aspirate TrypLE thoroughly and wash with 1× PBS. Add 1 ml of FINE media, gently detach cells using a cell scraper and dissociate clumps to single cells with the 1 ml pipette. Seed cells at high ratio of 1:2 in coated plates and transfer to a hypoxia (5% O₂, 5% CO₂) incubator for subsequent culture. Media is refreshed daily. FINE culture cells are subsequently passaged at 1:2 to 1:4 ratio. For most cell lines, differentiated cells are observed in the first 2-3 passages and gradually decreased over passages. For experiments described, FINE cells are cultured in media for at least 5 passages before use, unless otherwise described.

FINE PD03 Low Culture

FINE low PD03 cells were adapted from FINE conditions at passage 12. PD0325901 (Sigma) concentration was reduced from 1 μM to 0.3 μM.

RSeT Culture

H1 mTeSR cells were adapted to RSeT feeder-free culture conditions following manufacturer's protocol (STEMCELL Technologies). Cells were cultured in hypoxia conditions (5% O₂, 5% CO₂).

Small Molecules Treatment

3iL and 4 iLA media were supplemented with small molecule compounds at various concentrations for single and combinatory treatments. Small molecules used in the study: Dasatinib (Selleckchem), AZD5438 (TOCRIS), CHIR-98014 (Sigma), Crenolanib (Selleckchem), Saracatinib (Selleckchem), Src Inhibitor-1 (Sigma), Nilotinib (STEMCELL Technologies), Imatinib (STEMCELL Technologies), Dinaciclib (Selleckchem), WH-4-023 (Sigma).

High-throughput Small Molecule Screen

3,000 cells cultured in mTeSR1 (STEMCELL Technologies) or 3iL were seeded per well into 384-well plates (Greiner) coated with 30× Matrigel (Corning) or 30× growth factor reduced Matrigel (Corning) in 45μ1 of medium. 4 hours after seeding, cells were treated with anti-cancer and anti-kinase libraries (Selleckchem; kinase inhibitor screening library—customised collection of 273 kinase inhibitors, anti-cancer compound library—customized collection of 349 bioactive compounds). Small molecules were used at 3 different concentrations: 100 nM, 1 μM and 10 μM for each condition. 48 hours after the treatment, culture media was renewed concomitant with a second round of treatment with the compounds. 48 hours after the second round of treatment, cells were fixed with 4% formaldehyde (Sigma) and stained with Hoechst 33342 dye (1:4000, Invitrogen). Images were taken using Opera Phenix High-Content Screening System (PerkinElmer) at 20× magnification. Images were processed and fluorescence signal was quantified using Columbus Image Data Storage and Analysis System (PerkinElmer). Screen analyses was done using Screensifter software(Kumar et al., 2013). Z-score for zsGreen fluorescence was calculated using formula: z=(X−μ)/s.d. where μ—mean, s.d.—standard deviation of whole population, X−integrated intensity of zsGreen divided by total number of cells.

RNA-seq Analysis

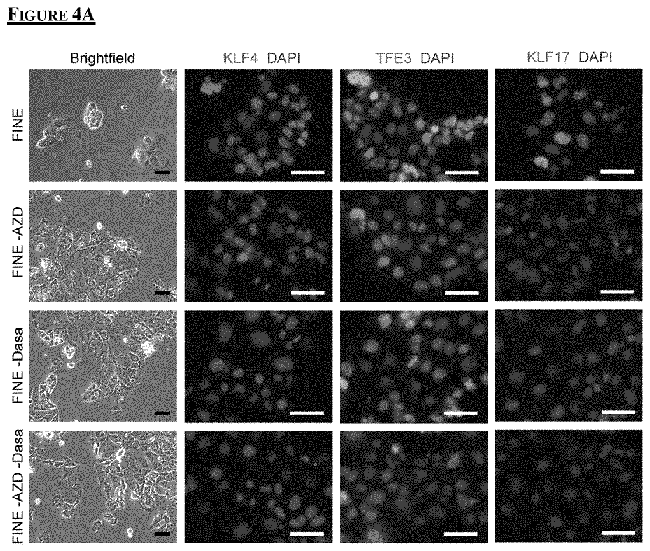

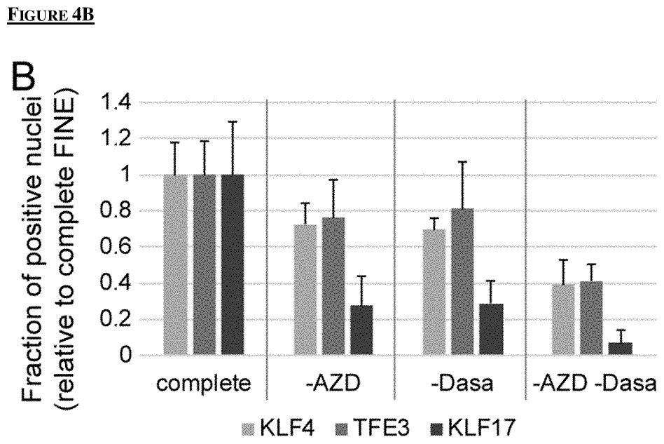

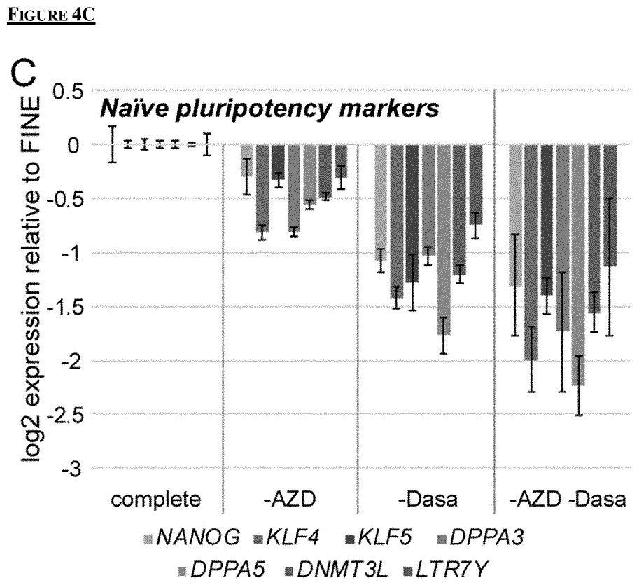

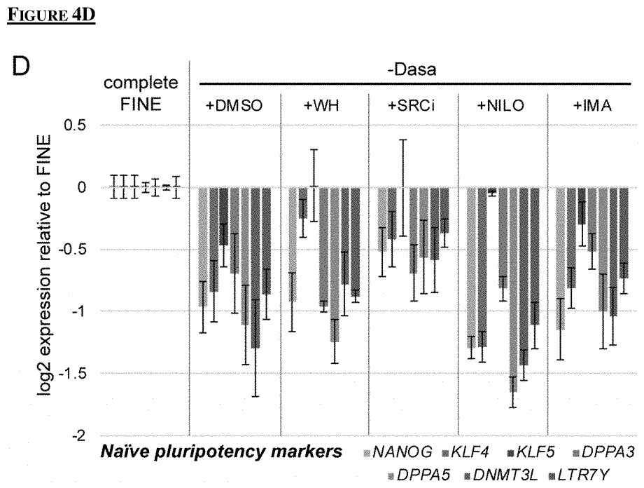

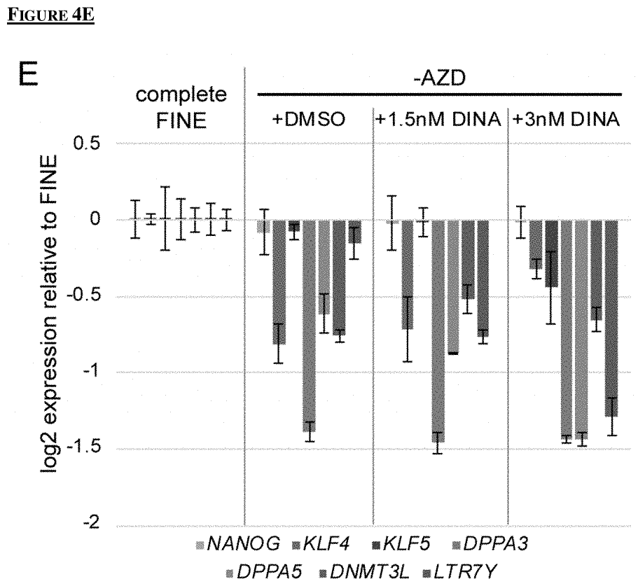

RNA-seq data were mapped against the human genome version hg19 with STAR-2.5.2b (Dobin et al., 2013). R-3.4.1 (R Development Core Team, 2014) and Bioconductor 3.6(Gentleman et al., 2004) were used for the RNA-Seq analysis. Reads were counted using the R package GenomicAlignments (Lawrence et al., 2013) (mode=‘Union’, inter.feature=FALSE), only primary read alignments were retained. Rlog transformed values of the counts, sample normalization factor of the samples, and differential expression values of genes were calculated using DESeq2 (Love et al., 2014). Plots in FIG. 4 were created using ggplot2_2.2.1 (Wickham, 2016).

Normalized values of repeats were calculated by dividing read counts to both sample normalization factor and per kb of the repeat.

For every stages of single cell data, Wilcoxon test is performed against the other stages in order to find the differentially expressed repeats. Afterwards the p-values were corrected by using Benjamini & Hochberg method. Significantly expressed repeats should have at least average 20 RNA-Seq reads in one of the development stages, log 2 change value should be higher than 1 (or lower than -1), and their adjusted p-values should be smaller than 0.05. RNA-seq data have been deposited in GEO under accession number GEO: get number E-MTAB-8216.

RNA Extraction, Reverse Transcription and qPCR

Total RNA was extracted using TRIzol reagent (Invitrogen) according to manufacturer's protocol followed by DNAase I treatment (Ambion).

250-1000 ng of DNAase treated RNA was reverse transcribed using SuperScript II (Invitrogen) and oligo-dT primers (Invitrogen) according to manufacturer's instructions. Reactions were performed in final volume of 20μ1. cDNA was diluted before qPCR analysis.

TABLE E3

| Gene | | Catalogue | Application and |
|---|---|---|---|
| name | Company | number | dilution |
| OCT4 | Abeam | ab19857 | IF (1:5000) |
| NANOG | R&D | AF1997 | IF (1:100) |
| KLF4 | Santa Cruz | sc-20691 | IF (1:400) |
| KLF17 | Sigma | HPA024629 | IF (1:500) |
| CD75 | Abeam | ab77676 | IF (1:100) |
| TFE3 | Sigma | HPA023881 | IF (1:700) |
| Ki67 | BD Pharmingen | 550609 | IF (1:300) |
| H3K9me3 | Active Motif | 39765 | IF (1:500) |
| CD75 - eFluor 660 | eBioscience | 50-0759-42 | FACS 5ul/test |
| CD130 - PE | BD Biosciences | 555757 | FACS 20ul/test |

List of antibodies used in this study

qPCR was performed using KAPA SYBR FAST master mix (KAPA Biosystem) following standard procedures. qPCR reactions were performed in biological duplicates or triplicates in 384-well plates on the ViiA™ 7 Real-Time PCR System (Life Technologies). Two technical replicates were carried out for each qPCR reaction and data was normalised to GAPDH. The relative abundance of transcripts was calculated using ΔΔC(T) method. Primer sequences used in this study are listed in Table E3 (below).

Immunofluorescence

Before fixation with 4% formaldehyde (Sigma) cells were washed with PBS (Gibco) in tissue culture plates (Falcon) for 30 minutes at room temperature. Permeabilization was performed with 1% of Triton-X 100 in PBS followed by blocking step performed in blocking buffer (blocking buffer—8% FBS in PBS-T (PBS-T 1% Tween in PBS)) each for 30 minutes at room temperature. Cells were incubated with primary antibodies, diluted in blocking buffer, overnight at 4° C. with gentle agitation. Primary antibodies details used in the study are provided in Table E4 (below). Cells were washed three times with PBS-T, following 2 hours incubation in room temperature with secondary antibodies (Alexa Fluor-couple, Invitrogen) followed by washes with PBS-T. Nuclei were counterstained with DAPI or Hoechst. After washing three times images were taken using Zeiss Axiovert Epifluorescence microscope. Images were processed using ImageJ and Illustrator. Antibodies used in this study are listed in Table E4, below.

TABLE E4
Sequences of qPCR primers used in this study

| Gene name | Primer sequence Forward | Primer sequence Reverse |
|---|---|---|
| POU5F1/OCT4 | CTTCGCAAGCCCTCATTTCACCA | GCACTAGCCCCACTCCAACCTG |
| NANOG | TTCTGCTGAGATGCCTCACACGG | TCTTGACCGGGACCTTGTCTTCC |
| SOX2 | AACCCCAAGATGCACAACTC | CGGGGCCGGTATTTATAATC |
| PRDM14 | GATGGCGCCTCCCTTGCTGA | CGCAGGGGGCGGTGGAATTA |
| LTR7Y | GCCATTTTATAGGATTTGGGAAG | TAACTGATGACATTCCACCATTG |
| HERVH | GCCTCTGCTCCTCCACCCTATAA | CGTTTAGCTCCAGCCACCTTTTT |
| KLF2 | CACCAAGAGTTCGCATCTGAAGG | TACATGTGCCGTTTCATGTGCAG |
| KLF4 | CTGGGTCTTGAGGAAGTGCTGAG | GTGGCATGAGCTCTTGGTAATGG |
| KLF5 | TCAGACAGCAGCAATGGACACTC | GTGGCCTGTTGTGGAAGAAACTG |
| KLF17 | GGGATGGTGCGATAGATTCA | GCCTCACCCTCACCTAACAA |
| DPPA3 | ATCGGAAGCTTTACTCCGTCGAG | CCCTTAGGCTCCTTGTTTGTTGG |
| DPPA5 | ACATCGAGCAGGTGAGCAAGG | CATGGCTTCGGCAAGTTTGAG |
| DNMT3L | CTGCGGAAGTCTCCAGGTTCA | GTAGCATCGGGTGCAATCAGG |
| GATA2 | GGTGCCCATAGTAGCTAGGC | GACAAGGACGGCGTCAAGTA |
| GATA6 | AGCCCAGGCTGCAGTTTTCCG | AGTCAAGGCCATCCACGGTCC |
| GAPDH | GGCTGTGGGCAAGGTCATCCCTGAG | GTCGCTGTTGAAGTCAGAGGAGACCACCTG |
| THOC2 | GCCACCGGACTTAACCAAGA | CTGTGCTTGTCCGAGGACTT |
| HUWE1 | ACTGGTGCAACTTCCTCCTTC | CCAAGTGCAGCTCCCATTCT |
| ATRX | ATGTAGGTGGTGTGCGGAAG | ACAGCATCCATCGCTCGAAA |
| ZSCAN4 | CACCAGAGAAGACACAGGAATG | ATGCACCCGTAGGTCTGATA |
| KDM4E | CAGGGAGGTGTGTTTACTCAAT | GTGTGGCGGAGTCTGATATTT |
| MBD3L2 | AACCTGCGTTCACCTCTTT | GCCATGTGGATTTCTCGTTTC |

Virus Production

Virus packaging was performed using the third-generation viral packaging system with plasmids: pMDLg/pRRE (Addgene # 12251), pRSV-Rev (Addgene # 12253), pMD2.G (Addgene # 12259). HEK-293T cells were transfected using Lipofectamine2000 (Invitrogen). Briefly, culture medium was changed 8h post-transfection and virus-containing supernatant was collected 30-56 h post-transfection. Supernatant was filtered through a 0.45 mm filter. Virus was concentrated using filter units following manufacturer's instructions (Amicon Ultra-15 Centrifugal Filter Units). For virus transduction, cells were seeded at 30-40% confluency 16-24 h before infection. Cells were transduced with the lentivirus in the presence of 4 μg/ml Polybrene (Sigma).

Reporter Line Generation

LTR7Y element (chr17:32,515,593-32,516,013, hg19) was cloned into modified pLVTH-zsGreen plasmid (Addgene # 12262). LTR7Y element was inserted between PacI and SalI cloning site, replacing Efl-alpha promoter. H1 hESCs were seeded at clonal density and transduced with lentivirus in presence of 4 m/ml of Polybrene (Sigma) for generation of LTR7Y-zsGreen reporter cells. Cells were re-seeded as single cells for generation of clonal lines for further study.

850 k DNA Methylation Profiling

Genomic DNA was isolated by DNeasy Blood & Tissue Kit (Qiagen) kit and processed using Zymo EZ DNA Methylation kit (Zymo Research Corp., CA, USA) following the manufacturer's recommendations for bisulfide conversion. Infinium® MethylationEPIC BeadChip (Illumina Inc.) was used to interrogate the genome wide methylation profile following the Infinium HD Methylation Assay Protocol.

The resulting raw data were normalized and processed using the ChAMP package under R statistical environment (v.3.1.1). The probes were aligned to the hg19 genome. Percentage of CG methylation was calculated by pooling all probes from individual chromosomes or different categories of genes. Pairwise methylation correlation plot was generated by linear regression model or Lowess weight model using methylation percentage from all probes in the sample. The chromosome and gene methylation track view was generated from Integrative Genomics Viewer (v.2.5.x).

FISH

Cells on 22×22 mm² coverslips were fixed with methacarn fixative (3 absolute methanol:1 glacial acetic acid) at room temperature for 10 mins. The cells were hybridized with custom synthesized Stellaris® RNA FISH probes (Table E5, below) and Human XIST with Quasar® 570 Dye (Cat nb: SMF-2038-1) (Biosearch Technologies) according to manufacturer instructions for hybridization of adherent cells.

TABLE E5
Customised FISH probes

| FISH | Probe sequence | Probe sequence |
|---|---|---|
| probe | (5′ to 3′) | (5′ to 3′) |
| HUWE1 | gaaccagtgagaaacgctgaag | cataaaggcagatagccaacac |
| | tatatgcaacgcctagtagtta | caacggacaagaaacgaggtgg |
| | aactattgatatttgcctattt | cagaaaaggtaggggaaagggg |
| | gtccgttctaatttaaatagtt | ctcgagaaaaaccagggtattc |
| | ttttccattctaatgcattgtt | ctaagccttagctctaaaaccg |
| | atccaatctggcttgatttgtg | gaagattagatgggacgacaga |
| | ccaacagtgtttctccaataaa | ttaaataccagcctcaactatc |
| | aagccaccaattttaactactg | aggacttaggctaaactcgaat |
| | aaaggccatatcattagttcta | tatggcaccatccacaaagatg |
| | acctgaatccatcttaatctaa | agacttgaggaaatggaaggct |
| | ctgtctccaggaataacatatt | cggatcagagtcatacaaacat |
| | attctggaagcggagcaaagag | agcagcatgcagagctaagaaa |
| | aaggctgtaccaattagccaaa | tcatagtttcgcttaatagtgg |
| | aaaatgactgggagtttttcgg | aaactgcaatatccaaacaccg |
| | ctcctaaaaaggagaaaggcgg | taatggccgtaaacgaaaaggc |
| | ggaaacatgagatatcgcgaga | ggggaggaatgaggaaggcaag |
| | cttcttaattcaccgcaggatg | tttactggagagttatcctcta |
| | cgttgagaactatcgcgatatt | tcagcagcaaaaatagatgtcc |
| | cacaacctaacgaagcagtgag | gaagtgagaagcaggtaagagg |
| | ctacgcgaagcgaaaagcaaat | tggaaaaggagtatggggagtg |
| | aaagccgaagtagctacagctt | ttatcttccttctaagggattc |
| | aacctctaccggacgggaaaag | aaggggtaaaatgtagtggagc |
| | gagaattctccggcttagaacg | acaatcaatgctgttttctagt |
| | aacgaatcccacgaggacgtaa | tgatagggaattaactgcctat |
| XACT | acatccaactacttacagtttc | ggtactaccattttgaatcatt |
| | acatacccactttcataatttt | aaacatgctgctctaagactat |
| | ttctaacactatttaattgccc | acttgattatattcagagtttt |
| | actggaatgatgattgcaatca | agatcattcaagtaagtctcaa |
| | atggtattccatgttattcgac | ggtgttacattatagccaatta |
| | tctttaaggtgataattcctga | atctggcagaaactctcattac |
| | atagcttaaggtactgaaagca | acacagtgtgttcattataacc |
| | agttttatagtacttacttggt | tactcagttactagcttcatta |
| | tcatttagatggcatccaaaga | gcaatggattctagtgaaatct |
| | tttctagctctactttgtgtaa | tcttaactgggctaccataaaa |
| | aaagtggcattttcaacctatt | ctggcagaattctaaactcata |
| | tttggataatacagcaaatgcc | tatggtttattaactactgaca |
| | atttctatgtgttgcagatgag | agtccttctgattttgtgaaag |
| | tggcaaataaaggaagctgaca | cttggacaaatcaacccaggag |
| | ggaagtcagggtgttaaaatgg | catgtggatggtcaaagaatct |
| | ggggactgaaaagtaaacattt | aaagaaagaacttgccagctgg |
| | gatgtatgagtagacatagctc | acaaaaccaggaatagtagaca |
| | aacagccacttttagttgaatt | gtagctgaaagtctgggaaaga |
| | cgttgttttatttcaatgttgt | ccagaacttatgactgtcaata |
| | caccgacaaattgttgcaattc | gaagatatgtggatagcagcat |
| | ctttaatgttgatggtgctaat | ttcatgtgagttactctctact |
| | gtacagttatgagtatatttcc | ccattaaaactgtccaagtctg |
| | tgctatgctattctctgaatta | ttaggatatatacagatatcca |

Briefly, 1 μL of reconstituted FISH probe stock was added to 100 μL of hybridization buffer (90 μL of Stellaris RNA FISH Hybridization buffer (Biosearch Technologies, cat# SMF-HB1-10) and 10 μL of deionized formamide) to make a working RNA FISH probe solution of 125 nM. Cells were washed with Wash Buffer A (2 mL of Stellaris RNA FISH Wash Buffer A (Biosearch Technologies, cat# SMF-WA1-60), 7 mL of nuclease-free water and 1 mL of deionized formamide) at room temperature for 5 min and incubated with RNA FISH probe solution in the dark at 37° C. for 16 h. Cells were then transferred to 6 well plate containing fresh Wash Buffer A and incubated in the dark at 37° C. for 30 min. Wash solution was aspirated and cells were incubated with DAPI nuclear stain (Wash Buffer A containing 5 ng/mL DAPI) to counterstain the nuclei in the dark at 37° C. for 30min. After that, cells were washed with Wash Buffer B (Biosearch Technologies, cat# SMF-WB1-20) for 5 min and then mounted onto glass microscope slides with mounting medium. Images were acquired by the automated slide scanner system (MetaSystems), using classifier MetaCyte SpotCount.Link.Quasar 570-670-63x-BIG. Images were then analyzed using the proprietary software Metafer 4 v3.11.8. A total of 250 cells were captured for each sample. Cells with poor probe hybridization were excluded from analysis and only cells with 2 spot staining present for control RNA FISH probe XACT were analyzed.

Teratomas

hESCs were dissociated with TrypLE Express (Life Technologies) and resuspended in 2× matrigel (Corning) diluted in DMEM:F12 (Nacalai Tesque) at the concentration cells 10⁶ cells/ml. 200 μl of cell suspension was injected into dorsal flanks of SCID nude mice. 4-8 weeks post injection, teratomas were surgically harvested for Mallory's Tetrachome staining.

Karyotyping

Various hESCs lines were seeded into glass cover slip slides as single cells. Karyotyping service including colcemid treatment and G-band analysis was outsourced with Parkway Laboratory Services.

Flow Cytometry Analysis

LTR7Y-ZsGreen, mTeSR1, 4 iLA +feeder and FINE cells were dissociated with TrypLE and resuspended as single cells in staining solution (2% FBS in PBS) with Thiazovivin (1 μM). Staining with CD75 and CD130 (Table E4) was performed on ice for 30min followed by washes.

Fluorescence intensity was analysed on BD LSRFortessa. FACS analysis was performed using FlowJo software.

RA Differentiation

mTeSR1 (STEMCELL Technologies) medium was supplemented with retinoic acid (Sigma, 10 μM) to induce exit from pluripotent state. Medium was refreshed daily. Cells were lysed for RNA work or FACS analysis 4 days after treatment.

CRISPR/Cas9 Targeting

Transfection of mTeSR1, FINE and 4 iLA+feeders cells with single plasmid co-expressing Cas9, gRNA and mCherry (GeneArt CRISPR EF1a-SpCas9-mCherry+gRNA) was performed using Mirus TransIT-LT1 (MirusBio). CRISPR/gRNA plasmids are gift from Meng How Tan laboratory. Cells were FACS sorted using BD FACS Aria II 48 h post-transfection for mCherry positive cells. DNA for PCR was extracted from sorted cells using QuickExtract (Epicentre) according to manufacturer's protocol. Genes targeted: EGFR (gRNA 1) and STAG2 (gRNA 2).

T7 Assay

PCR for T7 endonuclease assay was performed using Q5 High-Fidelity DNA Polymerase (NEB) with primers spanning region targeted by gRNA. T7 assay was performed according to manufacturer's protocol. Quantification was performed as previously described in (Ran et al., 2013).

Data and Software Availability

RNA-seq data have been deposited in GEO under accession number GEO: get number E-MTAB-8216.

### Example 2. Results: Small Molecule Screening for Conditions Supporting Maintenance of the Human Nave Pluripotent State in the Absence of Feeders

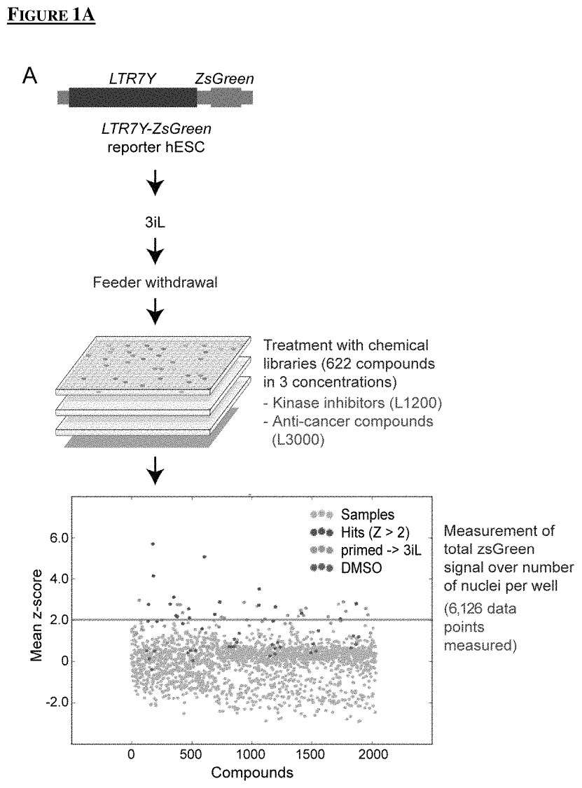

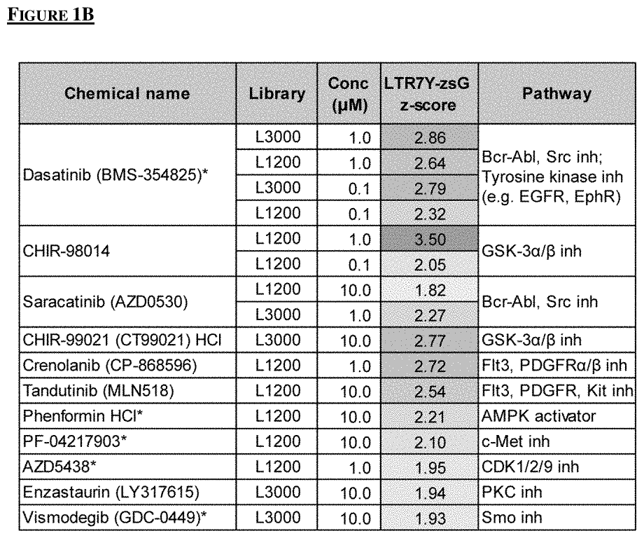

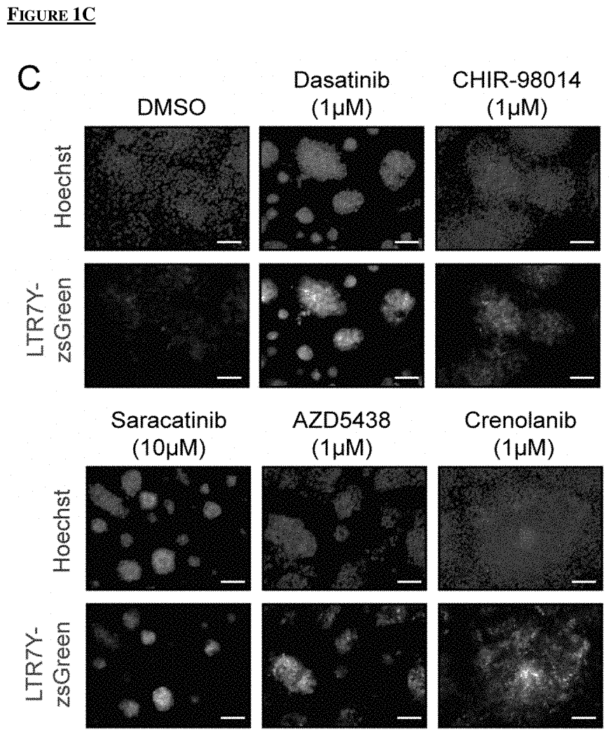

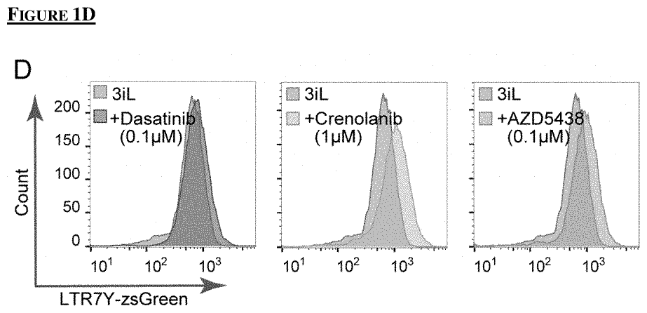

We sought a culture condition that would enable the propagation of naïve hESCs without feeders through a high-throughput small molecule screen (FIG. 1A). To visualize the naïve state, we developed a zsGreen reporter cell line driven by the ERV element LTR7Y, whose expression has been shown to be specific to pre-implantation blastocyst stage embryos (Goke et al., 2015).

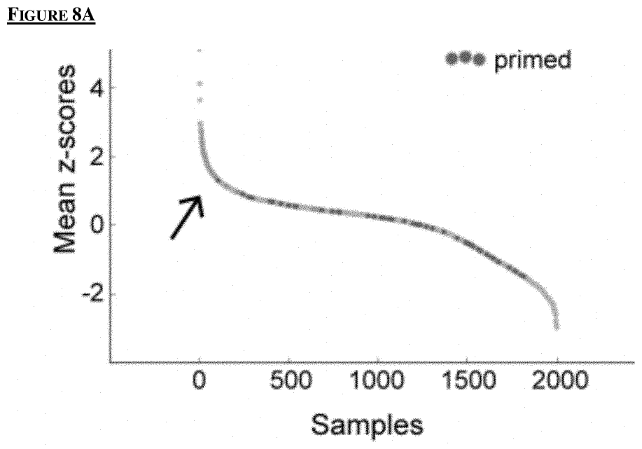

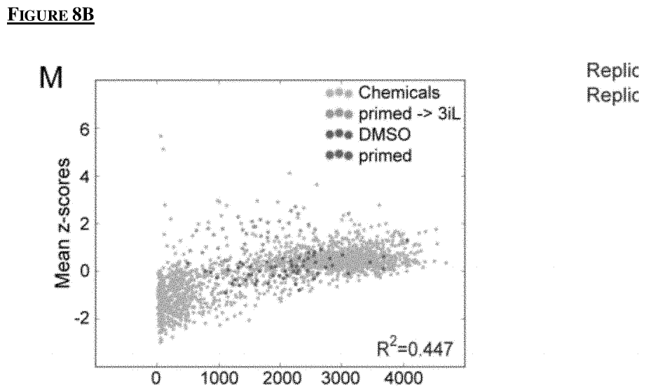

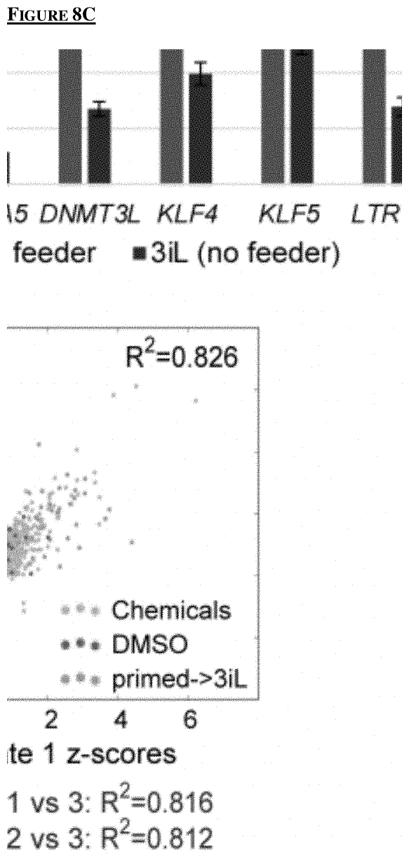

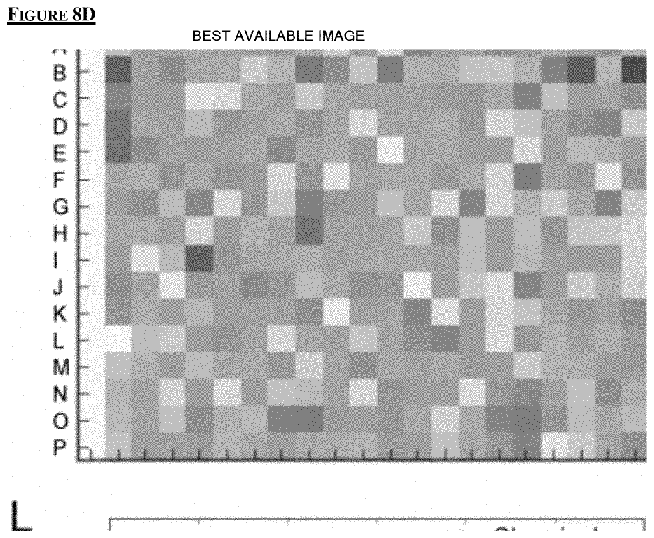

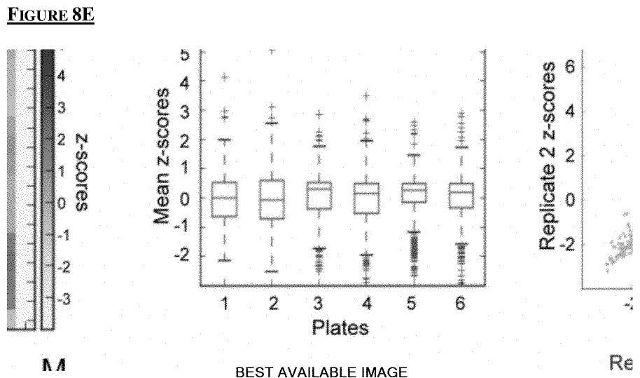

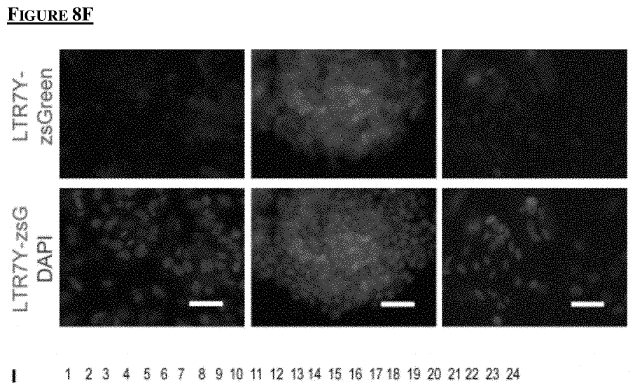

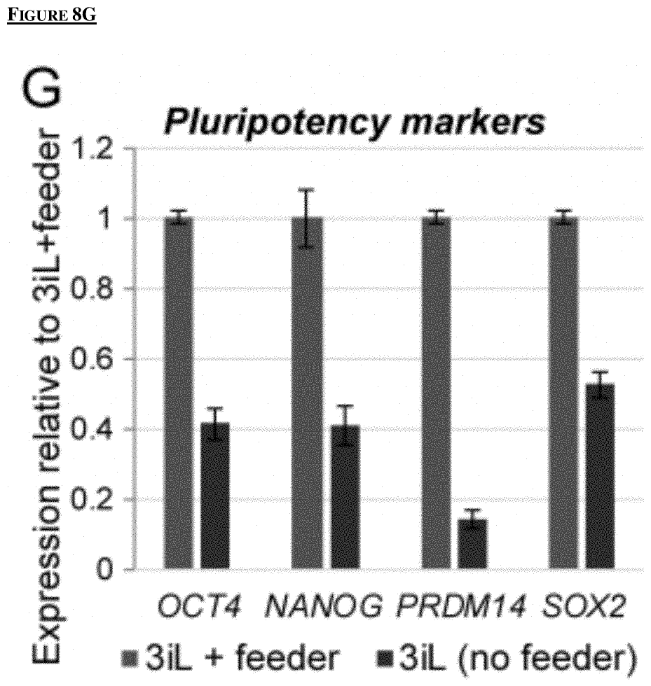

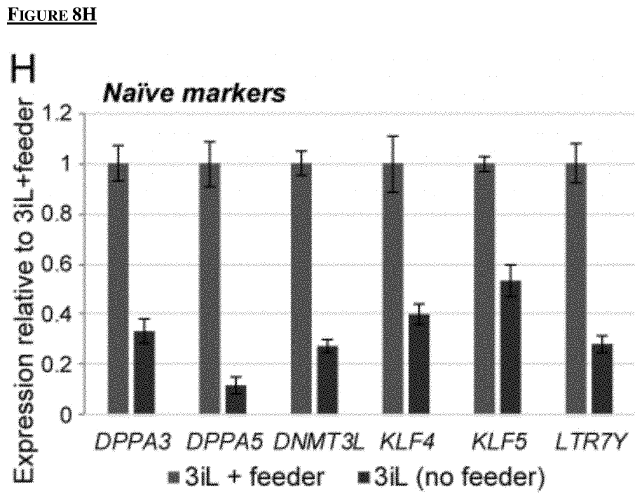

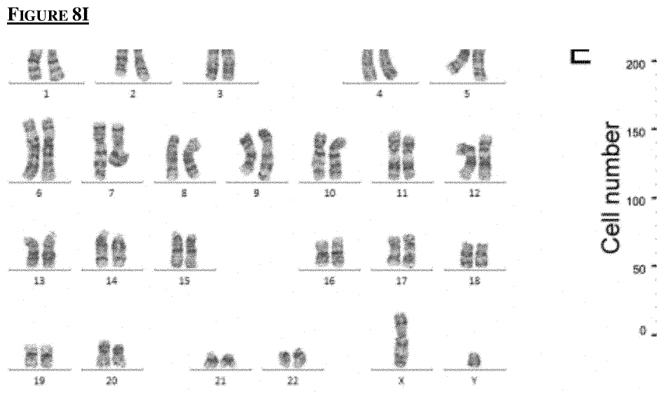

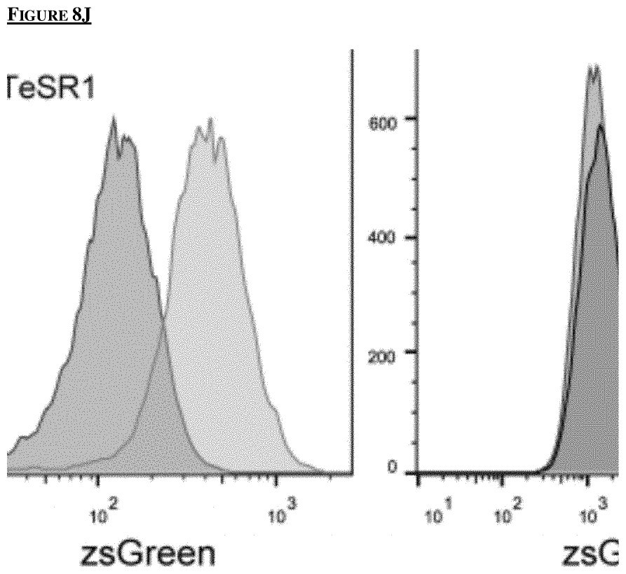

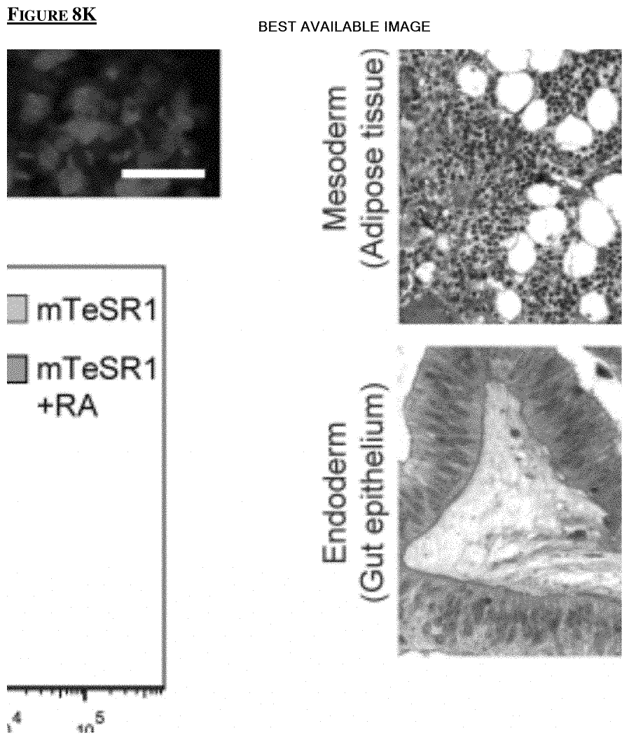

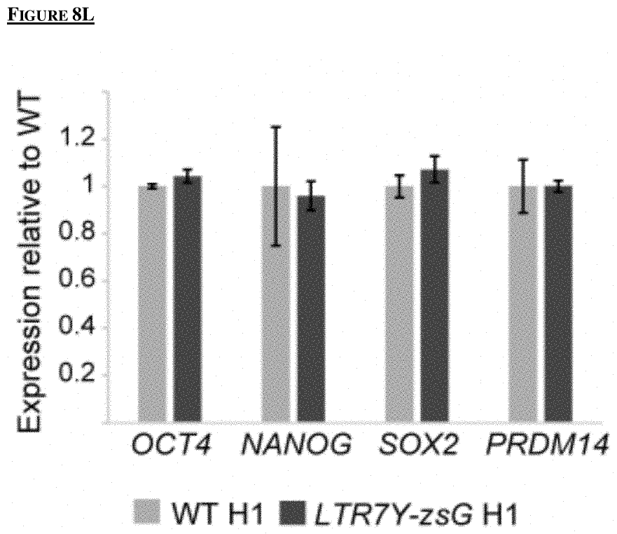

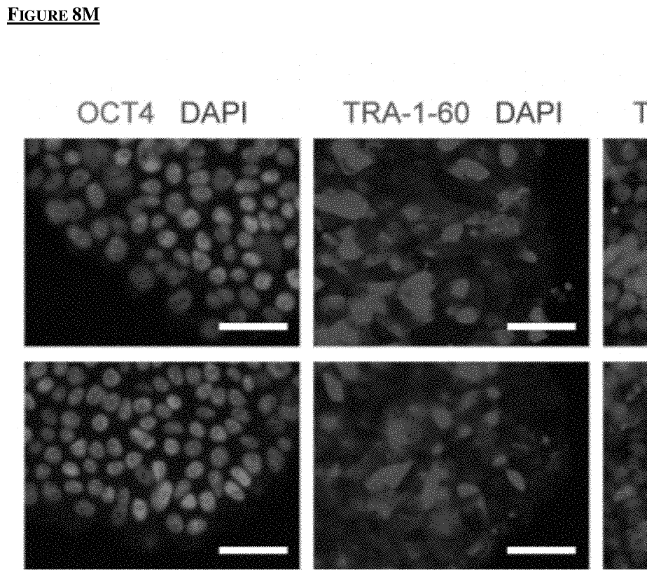

We confirmed that this reporter line is pluripotent (FIGS. 8A to 8C), is karyotypically normal (FIG. 8D), fluoresces only in naïve cells [3iL (Chan et al., 2013)] and not in primed or differentiated cells (FIG. 8E and FIG. 8F), and loses naïve markers and zsGreen fluorescence upon transfer to feeder-free culture (FIG. 8F to FIG. 8H).

To identify chemicals that can prevent collapse of naïve hESCs upon feeder withdrawal, we passaged hESCs cultured in 3iL (Chan et al., 2013) onto reduced Matrigel, and after attachment, added small molecules into the medium (FIG. 1A). For controls, we designated wells for hESCs treated only with DMSO vehicle, hESCs cultured in mTeSR (primed, thus showing baseline fluorescence), and primed hESCs freshly transferred to 3 iL (primed→3 iL; this initial conversion exhibits an increase in signal despite the absence of feeders). We screened a total of 622 compounds targeting signalling pathways governing embryonic development, cell proliferation and cell survival. The degree of preservation of the naïve state was measured through the average LTR7Y-zsGreen fluorescence intensity per cell, 4 days after feeder withdrawal. The screen was performed across 3 concentrations for each compound and in triplicate, summing up to 5,967 data points (Table E1, below).

We first ensured the quality of the screen by certifying the absence of intra-plate layout biases (FIG. 8I), proper inter-plate alignment (FIG. 8J) and good correlation between replicates (FIG. 8K). Z-scores were then calculated, and compounds that reproducibly scored above noise (z>2 in at least 2 replicates) were regarded as hits (FIG. 1B, S1L). We also detected no significant cell number bias in hit selection (FIG. 8M). Finally, we manually excluded compounds that auto-fluoresced in the green channel as false positives.

We observe that certain pathways are targeted by multiple compounds detected as hits (FIG. 1B), including those previously implicated in naïve pluripotency (e.g. GSK3, Src, PDGFR) (Takashima et al., 2014; Theunissen et al., 2014). Collectively, these ascertain that we rigorously and reliably identified compounds that could retain a naïve signature upon feeder withdrawal (FIG. 1B). Indeed, reporter activity of hits can be validated by visual inspection (FIG. 1C) as well as by flow cytometry (FIG. 1D).

In addition to small molecules targeting pathways implicated in naïve pluripotency, our screen identified novel regulators of naïve pluripotency such as Bcr-Abl/Src inhibitors Dasatinib and Saracatinib, as well as cyclin-dependent kinase inhibitor AZD5438 which warrant further investigation.

### Example 3. Results: Development of a Stable Feeder-free Human Naïve Pluripotent Culture Condition

The screen provided us a list of hits that can potentially substitute for fibroblast feeders in culturing naïve hESCs.

To identify which of the hits might be useful for long-term culture, we first tested the effect of short-term supplementation of individual hits on naïve pluripotency marker expression upon feeder withdrawal from published naïve culture protocols.

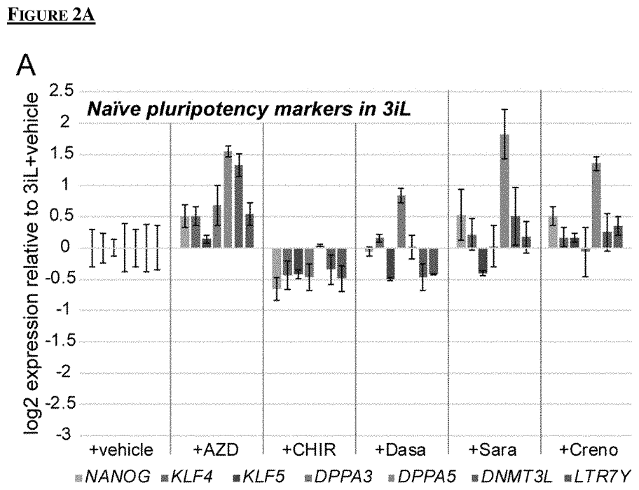

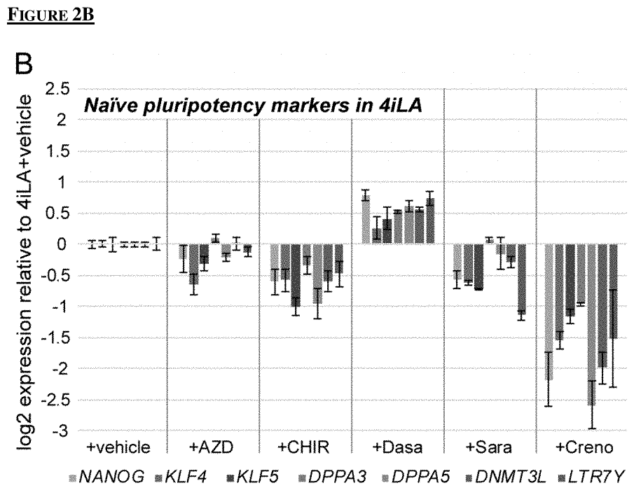

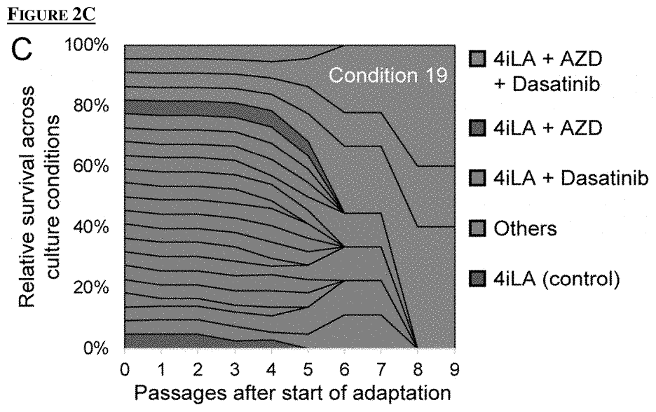

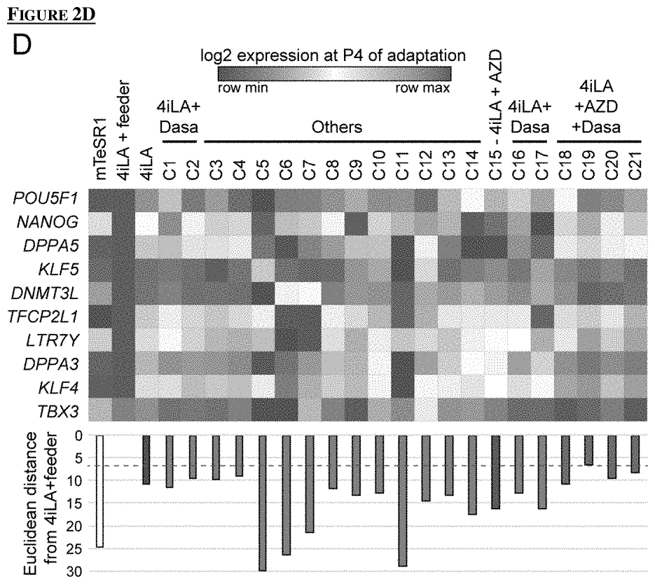

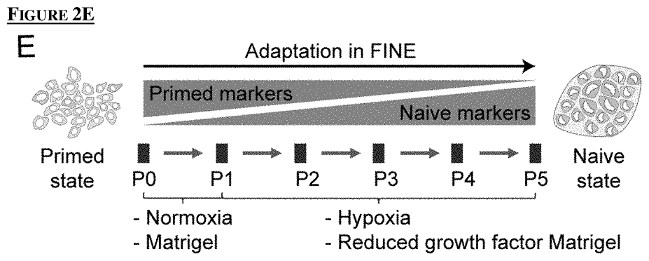

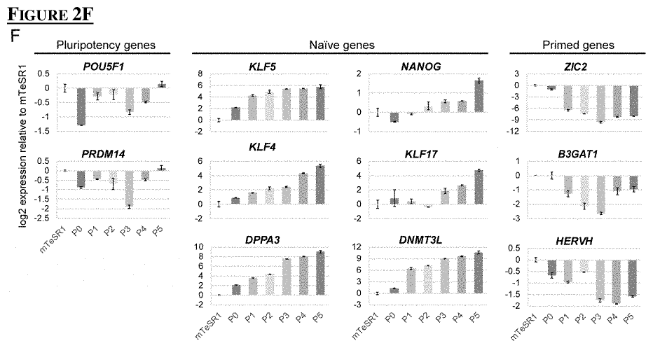

In 3 iL (Chan et al., 2013), only AZD5438 (AZD; CDK1/2/9 inhibitor) consistently attenuated downregulation of naïve pluripotency markers including LTR7Y (FIG. 2A), while in 4 iLA (Theunissen et al., 2016), only Dasatinib (Dasa; Bcr-Abl/Src kinase inhibitor) had the same effect (FIG. 2B).

We next sought to find a condition that enables stable naïve culture by adapting feeder-free primed hESCs onto media supplemented by one or more of the hits at various concentrations (FIG. 2C, Table E2, below).

TABLE E2
Formulations for the 21 conditions used to optimize
feeder-free naïve hESC culture, related to Figure 2.

| Conditions: | Details: |
|---|---|
| Cl | 4iLA + Dasatinib 0.2 μM |
| C2 | 4iLA + Dasatinib 0.5 μM |
| C3 | 4iLA + SRCi |
| C4 | 4iLA + Dasatinib 0.5 μM -LIF |
| C5 | 4iLA + Dasatinib 0.5 μM - ActivinA |
| C6 | 4iLA + Dasatinib 0.5 μM - PD0325901 |
| C7 | 4iLA + Dasatinib 0.5 μM - SB590885 |
| C8 | 4iLA + Dasatinib 0.5 μM - bFGF |
| C9 | 4iLA + Dasatinib 0.5 μM + CHIR99021 1.0 μM |
| C10 | 4iLA + Dasatinib 0.5 μM + PD0325901 0.5 μM |
| C11 | 4iLA + Dasatinib 0.5 μM + SB590885 0.25 μM |
| C12 | 4iLA + Dasatinib 0.5 μM + PD0325901 0.5 μM + |
| | SB590885 0.25 μM |
| C13 | 4iLA + Saracatinib 0.5 μM |
| C14 | 4iLA + Saracatinib 0.2 μM |
| C15 | 4iL + AZD5438 0.2 μM |
| C16 | 4iLA + Dasatinib 1.0 μM |
| C17 | 4iLA + Dasatinib 2.5 μM |
| C18 | 4iLA + Dasatinib 0.5 μM + AZD5438 0.1 μM |
| C19 | 4iLA + Dasatinib 0.2 μM + AZD5438 0.2 μM |
| C20 | 4iLA + Dasatinib 0.2 μM + AZD5438 0.1 μM |
| C21 | 4iLA + Dasatinib 0.5 μM + AZD5438 0.2 μM |
| 4iLA control | 4iLA |

We tested more combinations including AZD5438 and Dasatinib due to their favourable effects on short-term feeder withdrawal (FIG. 2A-B). From this point onwards, we solely utilized 4 iLA (Theunissen et al., 2016) as our basal medium, since it is the transgene-free culture condition shown to resemble the in vivo epiblast most closely at the time of the experiment (Nakamura et al., 2016).

At passage 4, we collected RNA from all conditions and quantified transcripts of genes associated with naïve pluripotency (FIG. 2D), and found that condition 19 had the closest profile to 4 iLA hESCs on feeder as measured by Euclidean distance. Despite retained expression of many naïve pluripotency genes, culture in 4 iLA without any feeders or supplementary molecules had very few cells surviving at passage 5.

Upon culturing for 8 passages, only conditions with both Dasatinib and AZD5438 are still actively proliferating (FIG. 2C). Since condition 19 exhibits the best survival profile while maintaining naïve gene expression signature, we decided to focus on the long-term propagation of cells in this medium.

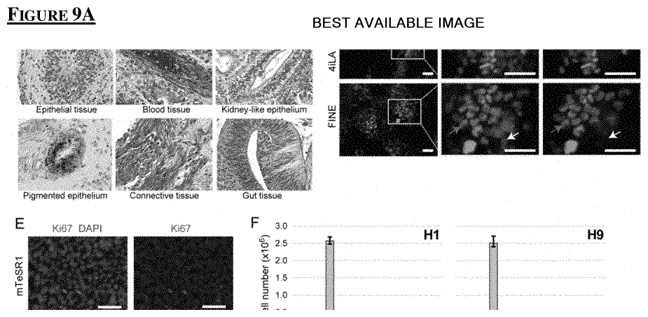

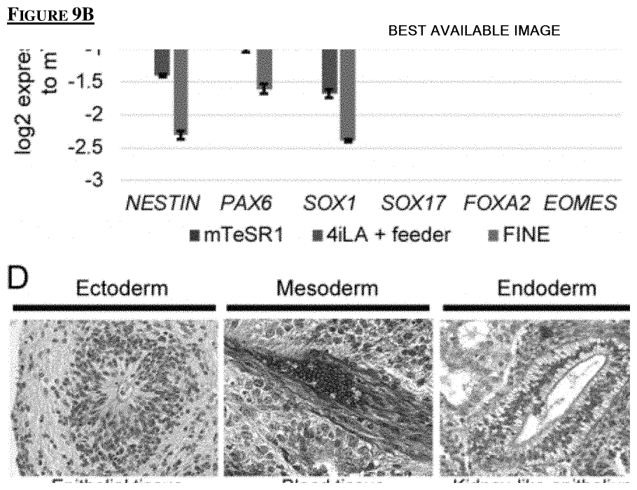

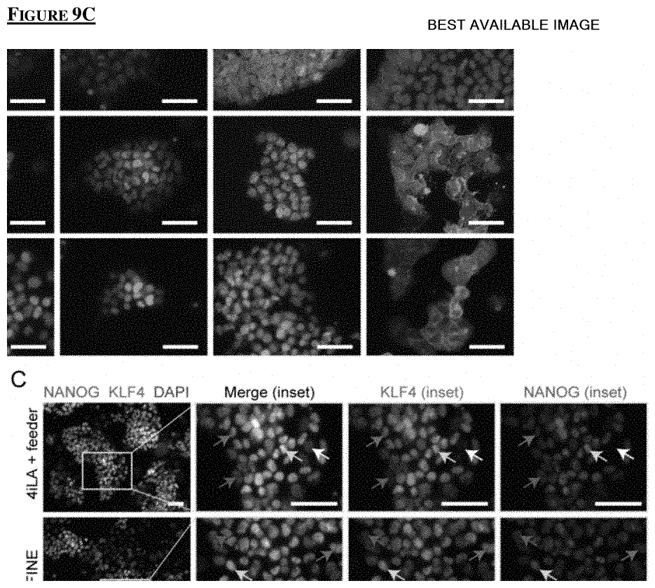

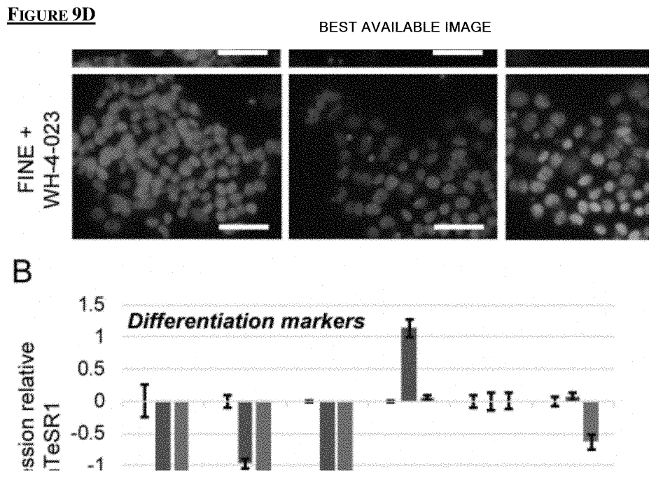

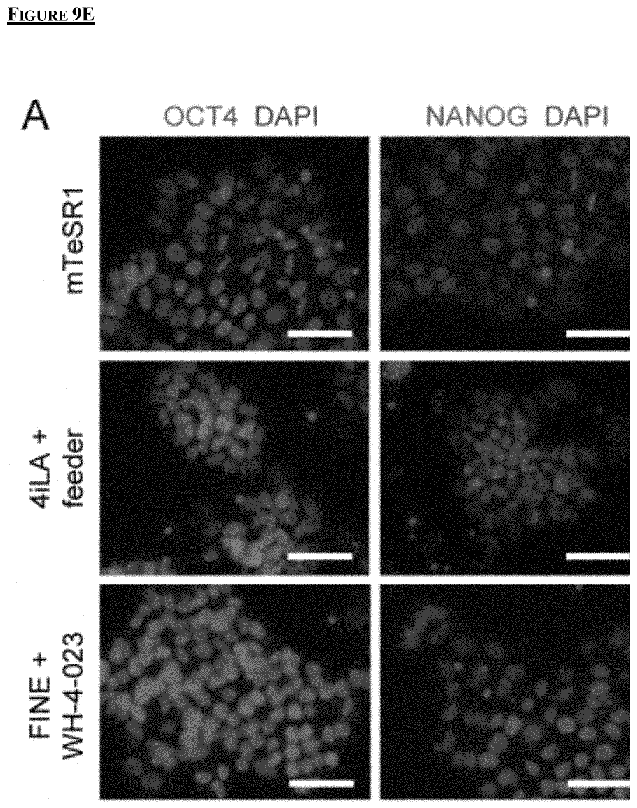

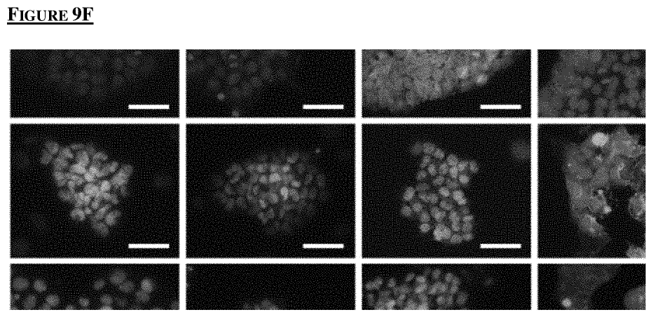

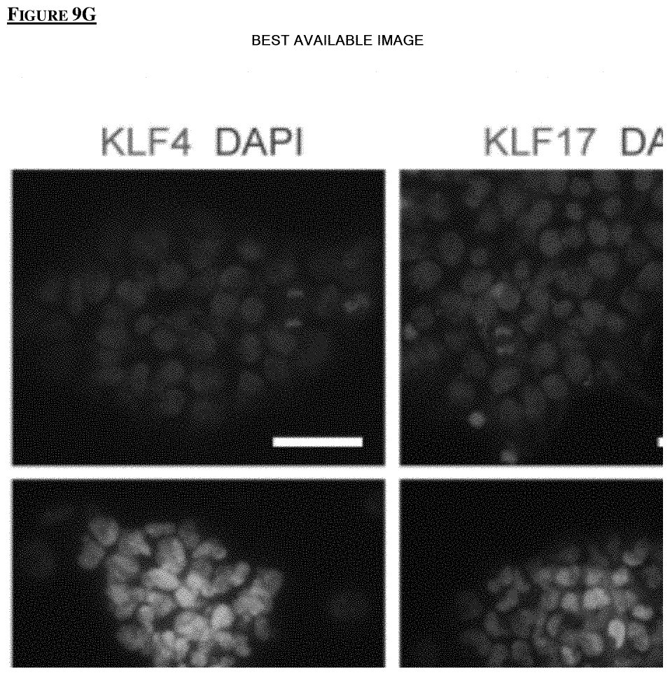

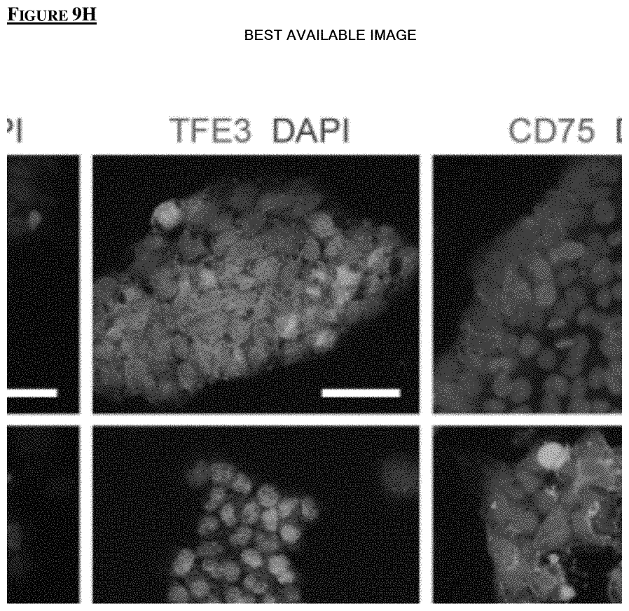

Further optimization showed that WH-4-023, a Src kinase inhibitor originally present in 4 iLA feeder-dependent culture (Theunissen et al., 2016) is dispensable for both adaptation and maintenance of feeder-free naïve cells (FIG. 9A), likely due to the presence of another Src inhibitor, Dasatinib (Araujo and Logothetis, 2010), in condition 19. Thus, we excluded it from the final formulation and called this feeder-independent naïve ESCs or FINE.

Our optimized protocol for adaptation in FINE culture conditions involves an initial conversion step from mTeSR1 medium on Matrigel to FINE under normoxia (P0), plus an additional 5 passages (P1-P5) under hypoxia on a reduced growth factor Matrigel substrate (FIG. 2E). During this course, human pluripotency markers such as POU5F1 and PRDM14 are transiently downregulated, but return to normal levels by P5 (FIG. 2F). Two trends of naïve-specific marker upregulation can be observed (FIG. 2F): those which are upregulated as soon as PO and gradually increase across passages (such as KLF4 and DPPA3), and those which are not upregulated until P3 onwards (such as NANOG and KLF17). Primed-specific markers are generally downregulated early on at P0-P1 (FIG. 2F). Taken together, adaptation in FINE does not require feeders at any step of the process, and the naïve pluripotent signature is established and stabilized by P5.

### Example 4. Results: Fine Cells Exhibit Hallmarks of Nave Pluripotent Cells

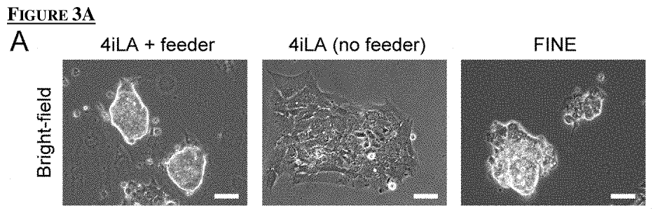

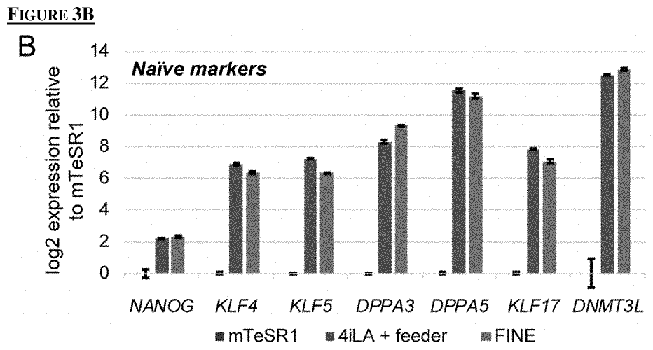

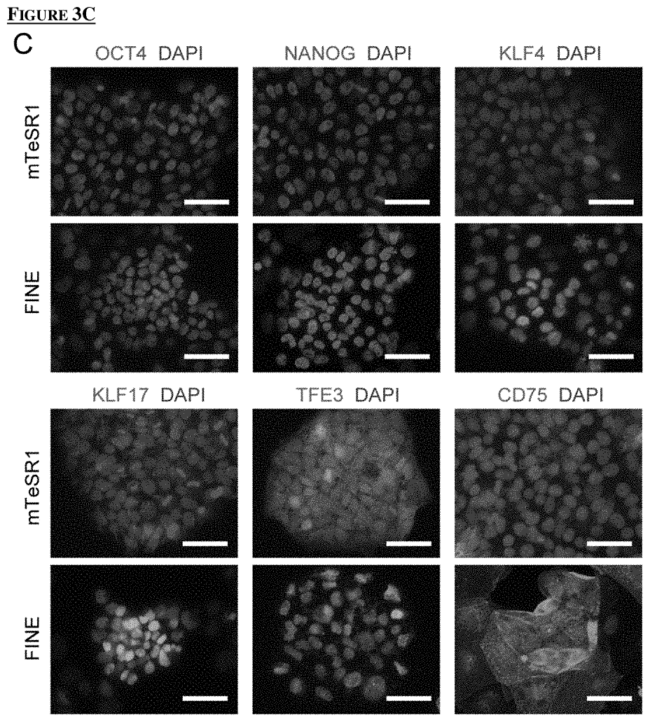

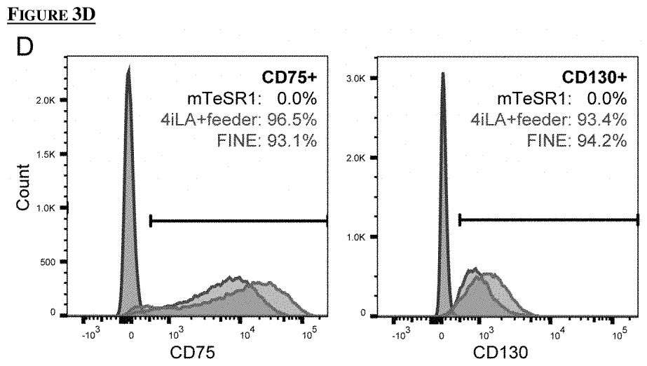

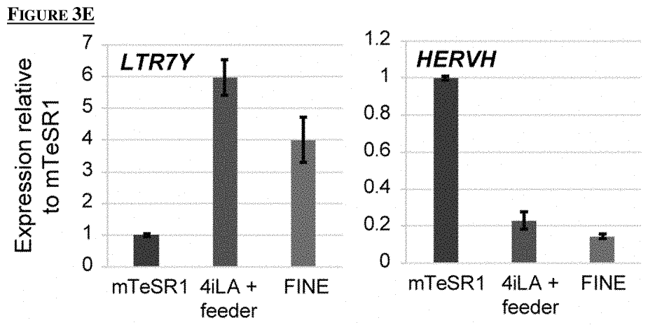

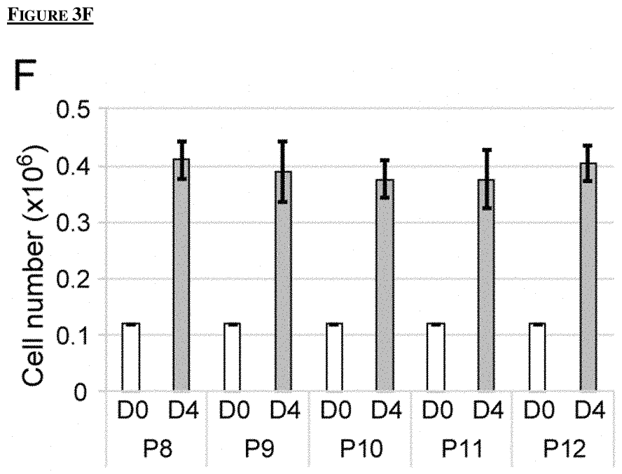

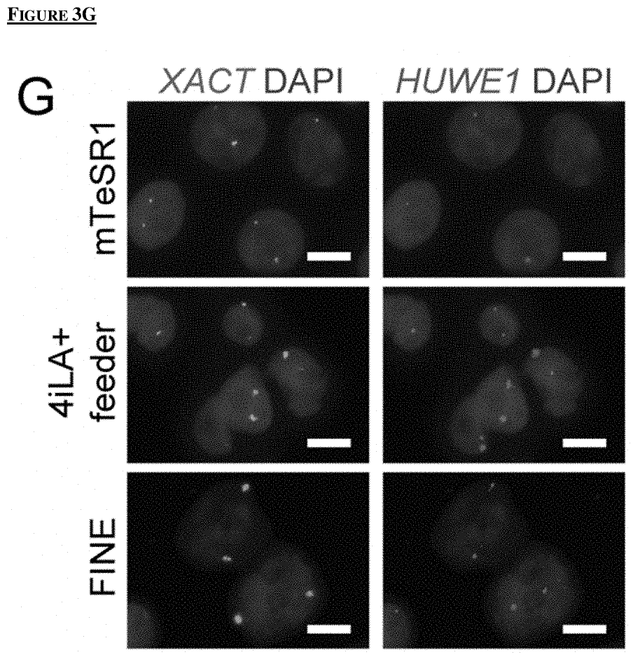

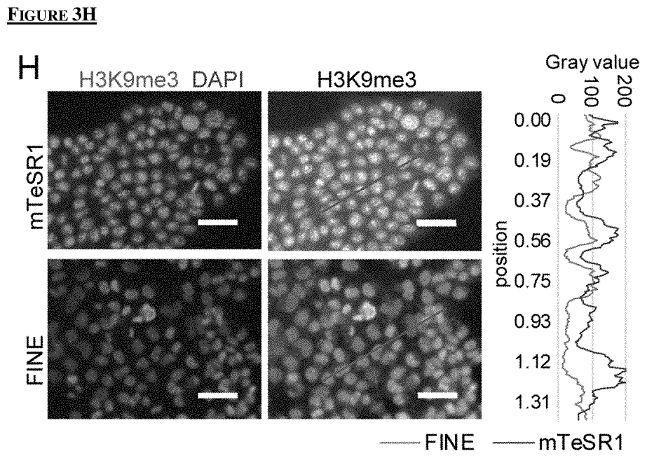

Throughout propagation (P5 onwards), FINE cells maintain compact morphology characteristic of naïve cells (FIG. 3A).

After sustained culture (>8 passages), we assessed the expression pattern of FINE compared to 4 iLA on feeders and observed comparable expression levels of blastocyst markers on both transcript and protein levels (FIG. 3B-C, S2A), and comparable or lower expression of lineage markers (FIG. 9B).

Nuclear localization of naïve-specific transcription factors KLF4, KLF17 and TFE3 is also observed in FINE, as in 4 iLA on feeders (FIG. 3C, S2A). Note that while some of these naïve transcription factors exhibit heterogeneous expression, the same is observed for 4 iLA on feeders (FIG. 9C). Nevertheless, naïve cells express naïve surface markers homogeneously (FIG. 3D), suggesting that all cells in culture are of naïve pluripotent identity, but transcription factor levels fluctuate as observed in non-ground-state naïve ESCs in mouse (Chambers et al., 2007; Hayashi et al., 2008; Niwa et al., 2009; Torres-Padilla and Chambers, 2014; van den Berg et al., 2008).

Importantly, expression of stage-specific ERVs LTR7Y and HERVH in FINE mimics levels that of 4 iLA on feeders (FIG. 3E) and functional pluripotency is preserved as demonstrated by teratoma formation (FIG. 9D).

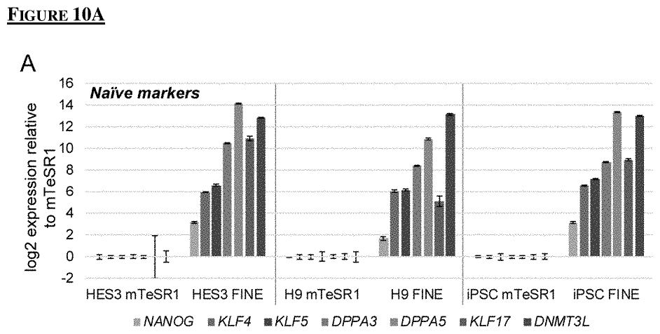

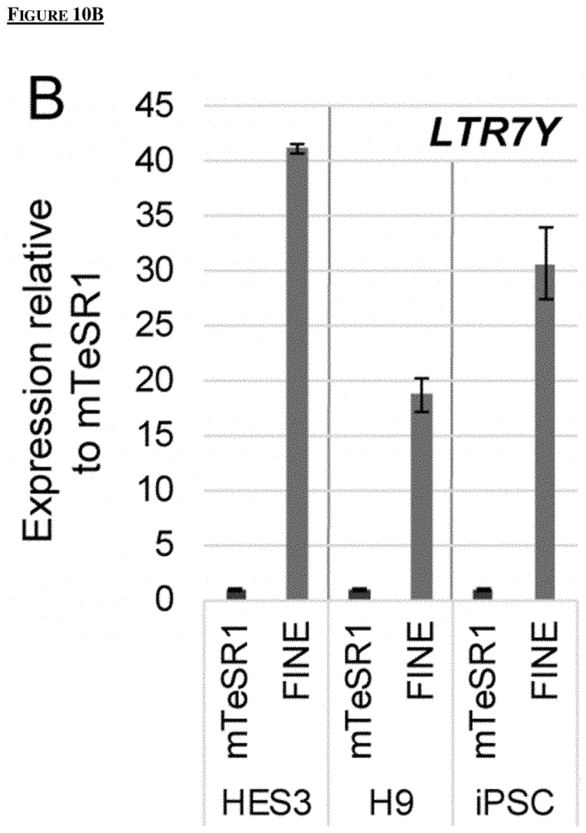

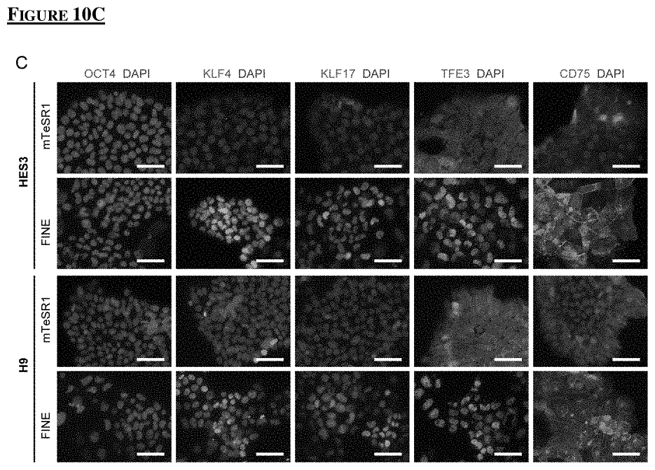

FINE conditions induce naivety similarly across multiple human pluripotent cell lines (FIG. 10A to FIG. 10C), confirming the robustness of this culture system.

To assess self-renewal capability, we quantified the cell number of FINE cells across passages and observed ˜4-fold propagation every 4 days (FIG. 3F), consistent with positive staining for proliferation marker Ki67 (FIG. 9E). This doubling rate (˜60 hr) is comparable to 4 iLA +feeder (60-96hr), but is slower than primed cells in mTeSR1 (˜22 hr) (FIG. 9F). X-chromosome status through in situ hybridization of the HUWE 1 locus (Sahakyan et al., 2017) indicate XaXa for both FINE and 4 iLA+feeder, while primed cells exhibit XaXi (FIG. 3G, S2G). Transcripts indicative of X-chromosome activation are similarly upregulated by approximately 2-fold or more in 4 iLA+feeder and FINE versus primed cells (FIG. 9H), consistent with the switch from monoallelic to biallelic expression in XaXa cells (Lin et al., 2007). We also observe that FINE cells exhibit lower levels of H3K9 trimethylation compared to primed mTeSR1 culture, typical of the higher proportion of euchromatin in ground-state naïve pluripotent cells (Tosolini et al., 2018) (FIG. 3H).

Taken together, these results indicate that FINE is a bona fide human naïve pluripotent culture system independent of feeder support.

### Example 5. Results: Fine Cells Are Dependent on Both Dasatinib and Azd5438

Our data showed that both AZD5438 and Dasatinib are crucial for the establishment of FINE cells. Therefore, we wanted to test if both are also important for the maintenance of naivety in FINE.

Withdrawal of either or both AZD5438 and Dasatinib caused dispersal of the compact morphology typical of naïve cultures, indicating exit from naïve pluripotency (FIG. 4A). This is corroborated by the reduction in nuclear staining of naïve-specific transcription factors (FIG. 4A-B), as well as the downregulation of pluripotency and naïve transcripts (FIG. 4C). Dasatinib withdrawal had a more pronounced effect in loss of naivety than AZD5438 withdrawal, but more importantly, markers were lost most significantly upon withdrawal of both compounds. These results indicate that these two compounds act on distinct pathways in parallel to maintain the naïve pluripotent state in the absence of feeders, and supplementation of either compound alone is insufficient to sustain naivety long-term.

Dasatinib is a kinase inhibitor with a broad range of targets including Bcr-Abl, Src family kinases and multiple receptor and non-receptor tyrosine kinase families (Li et al., 2010). We know that Dasatinib affects Src, as its addition allowed removal of WH-4-023 from the FINE formulation (FIG. 3C, S2A); However, replacement of Dasatinib with other Src inhibitors like WH-4-023 and SRCi, as well as other Bcr-Abl inhibitors like Nilotinib and Imatinib (FIG. 4D) failed to sustain feeder-free naïve hESCs. Similarly, replacement of AZD5438 with another multi-cyclin dependent kinase inhibitor Dinaciclib (which also inhibits CDK1/2/9, but also CDK5) did not sustain FINE cells (FIG. 4E). These results establish the essential role of these two compounds in sustaining feeder-free naïve culture, and imply that these compounds might have other unknown targets that confer such effects.

### Example 6. Results: Analysis of the Global Transcriptome of Fine Cells

To confirm the efficacy of FINE in converting hESCs to a feeder-free naïve state, we performed RNA-seq analysis on hESCs cultured in mTeSR1 (primed), 4 iLA with feeders and FINE.

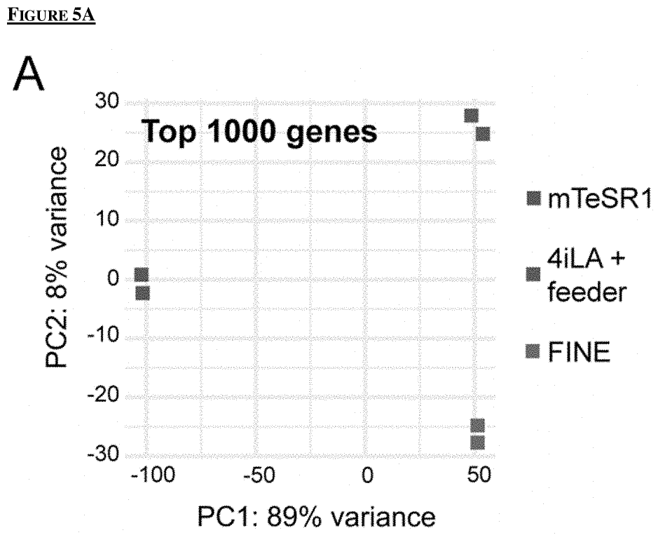

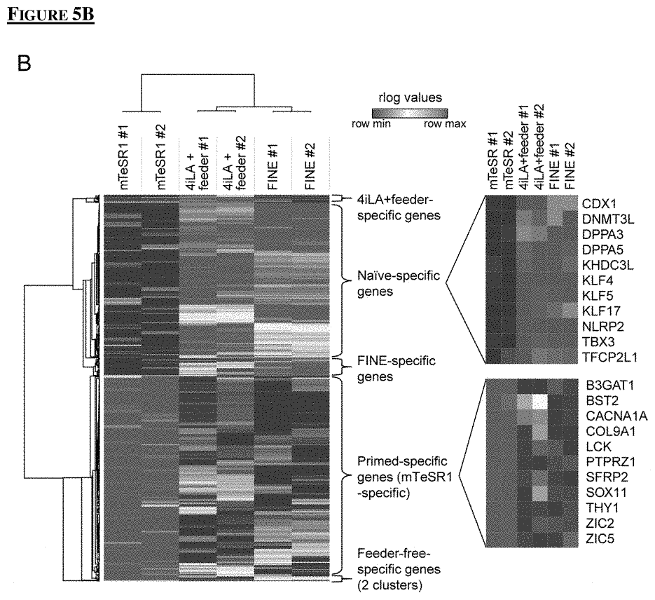

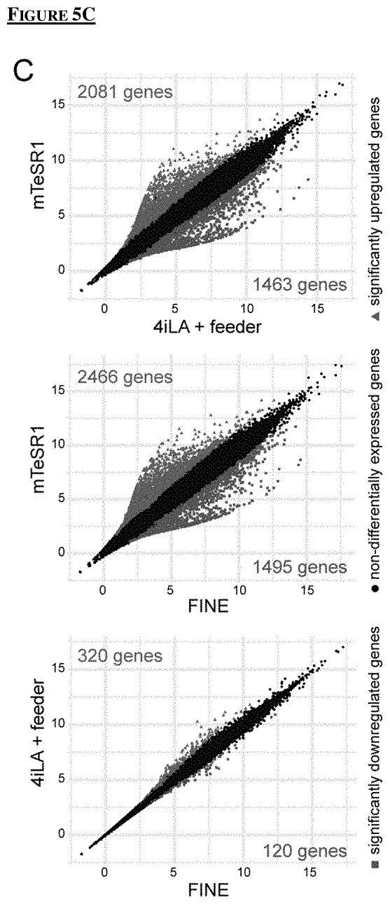

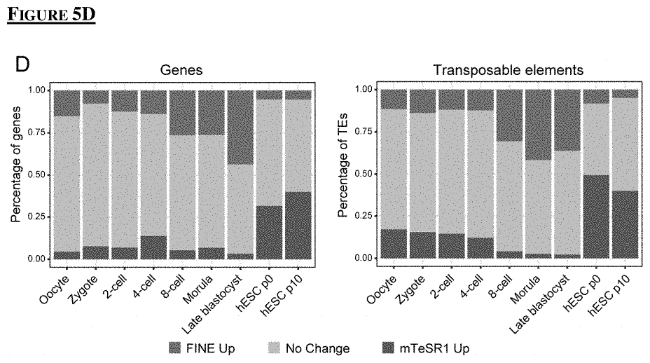

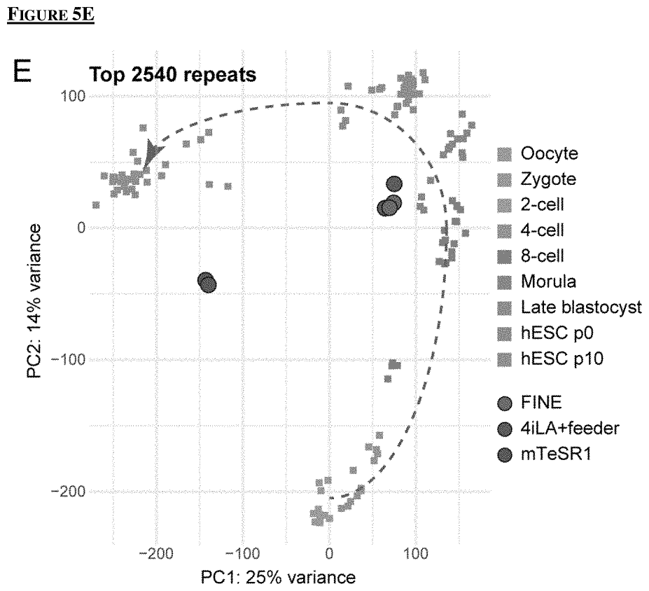

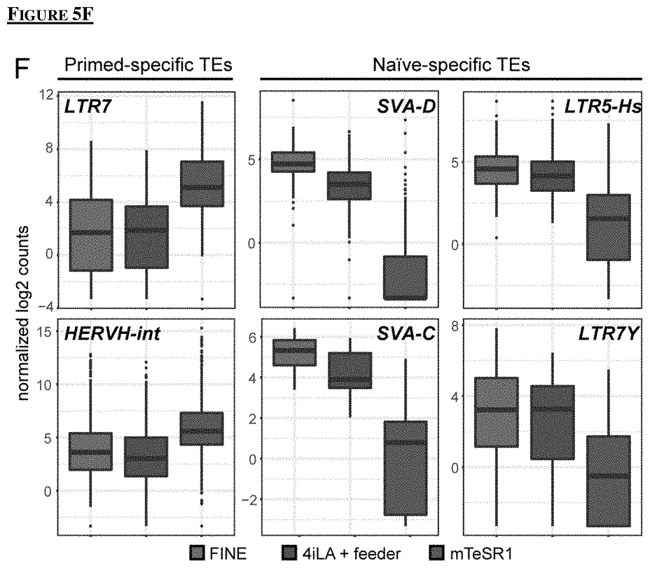

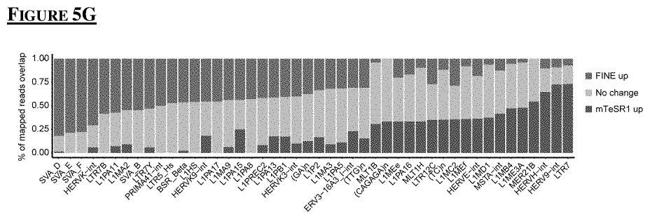

Principal component analysis and correlation confirms that FINE very closely resembles naïve cells on feeders (FIG. 5A, S4A). Analysis of the top 1000 differentially expressed genes show 6 major clusters, with the two biggest clusters comprising genes specifically expressed in either primed (mTeSR1) or naïve (4 iLA+feeder, FINE) states including well-known markers such as DNMT3L, DPPA5, KLF4 and TFCP2L1 (Chan et al., 2013; Takashima et al., 2014; Theunissen et al., 2014) (FIG. 5B).

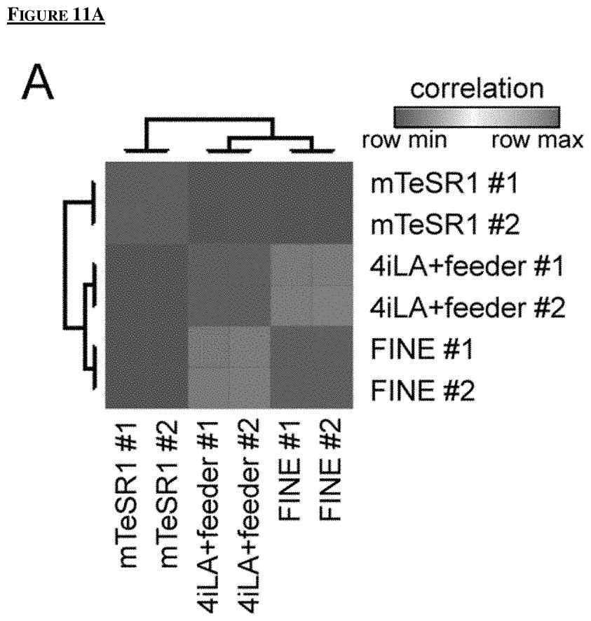

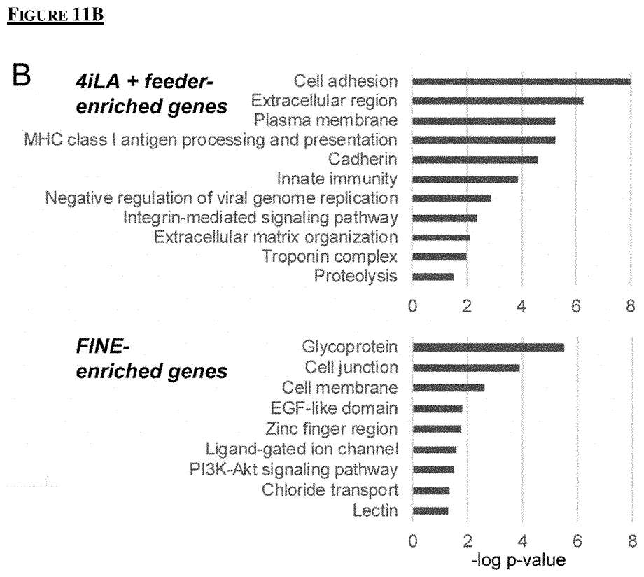

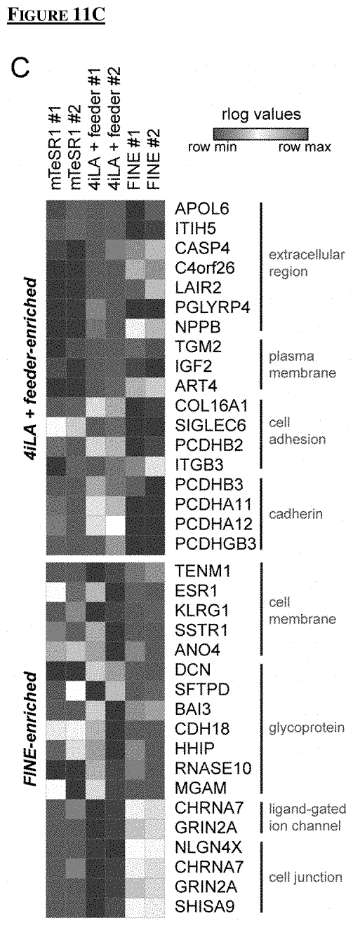

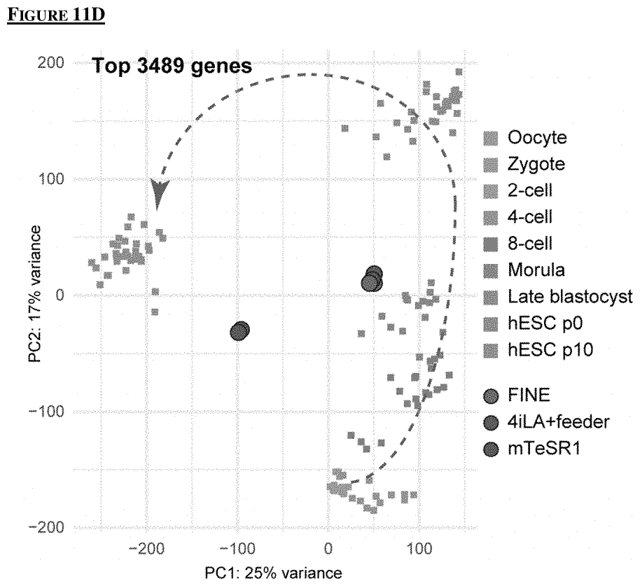

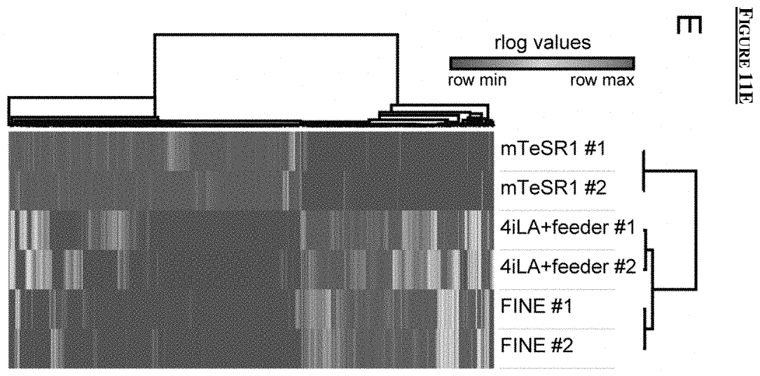

In fact, differential gene expression analysis between FINE and 4 iLA+feeder only generates 440 genes (FIG. 5C), most of which are involved in cell adhesion, cell-cell junctions and extracellular matrix interactions (FIG. 11B and FIG. 11C), reflective of the replacement of feeders with reduced Matrigel. Most of the FINE-enriched genes are also upregulated in feeder-free primed mTeSR1 culture (FIG. 11C), suggesting that these genes play a role in adaptation to feeder-independent in vitro culture. Allocation of differentially expressed genes between FINE and mTeSR1 onto published stage-specific genes of the human developing embryo (Xue et al., 2013; Yan et al., 2013) assigns FINE closest to the late blastocyst stage in vivo, and far from primed pluripotent cultures (FIG. 5D left, S4D).

Expression profiles of repetitive and transposable elements have been demonstrated to be highly stage-specific (Goke et al., 2015), and can be used as a sensitive barometer for matching pluripotent cultures with stages of in vivo early human development (Theunissen et al., 2016). Analysis of this ‘transposcriptome’ matched FINE cells with the 8-cell to blastocyst stages of the human embryo (FIG. 5D right, 5E), with FINE cells very closely resembling hESCs cultured in 4 iLA+feeder (FIG. 5E-F, S4E). Transposable elements upregulated in FINE versus primed cells include known naïve-specific families such as LTR5-Hs, LTR7Y and HERVK (Goke et al., 2015; Grow et al., 2015; Theunissen et al., 2016) (FIG. 5F-G).

Overall, FINE cells are transcriptionally equivalent to naïve pluripotent culture with feeders, and closely resemble the in vivo pre-implantation blastocyst.

### Example 7. Results: Analysis of Global DNA Methylation in Fine Cells

To examine the chromatin status of FINE cells, we profiled global DNA methylation status in FINE cells compared to mTeSR1 and 4 iLA+feeder cells.

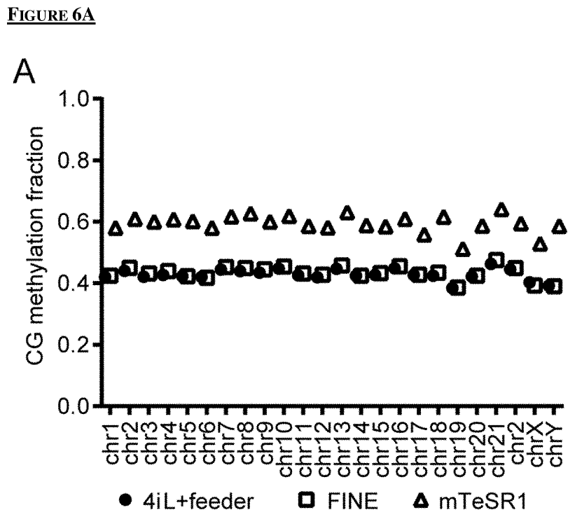

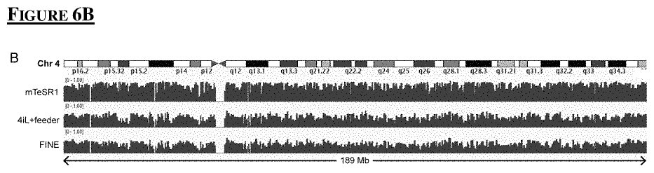

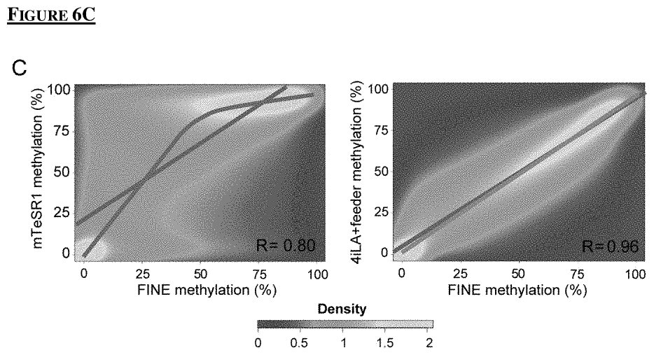

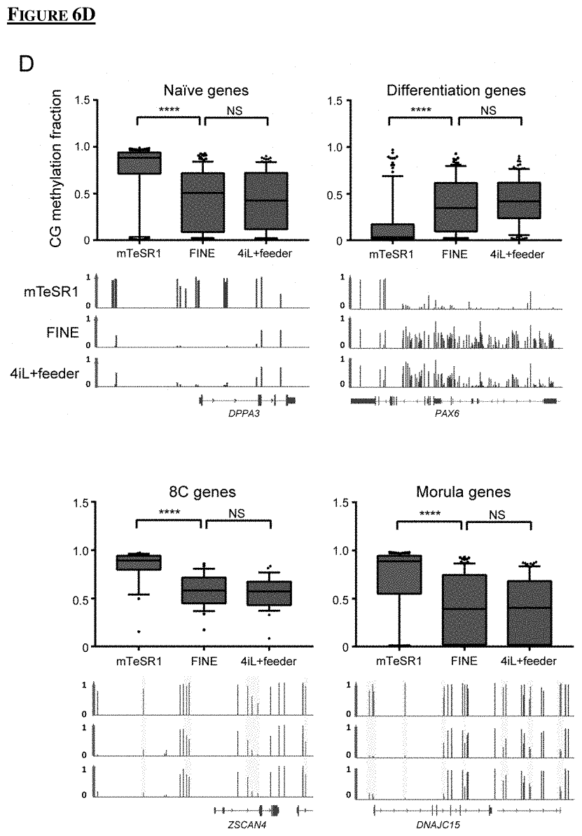

Across all chromosomes, the percentage of methylated CG sites is equivalently lower in cells cultured in both naïve conditions compared to primed hESCs (FIG. 6A-B), consistent with previous reports of global DNA hypomethylation in the naïve pluripotent state (Takashima et al., 2014; Theunissen et al., 2016). In fact, the methylated regions for FINE and 4 iLA+feeder are very highly correlated (FIG. 6B-C), corroborating that these naïve pluripotent states are equivalent.

Consistent with transcript expression patterns, DNA at naïve marker loci, as well as at 8C and morula-associated gene loci, are less methylated in FINE and 4 iLA+feeders, while differentiation genes display higher DNA methylation (FIG. 6D).

All in all, global DNA methylation profiling supports that FINE represents a feeder-free equivalent of 4 iLA-cultured naïve hESCs.

### Example 8. Results: Advantages and Applications of Fine Cells

To test the utility of FINE cells for applications such as genetic targeting, we compared the amenability of naïve cells to DNA delivery via transfection.

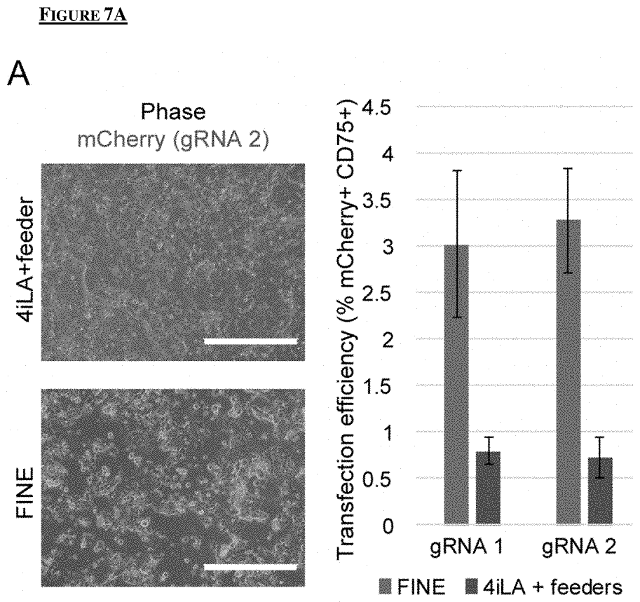

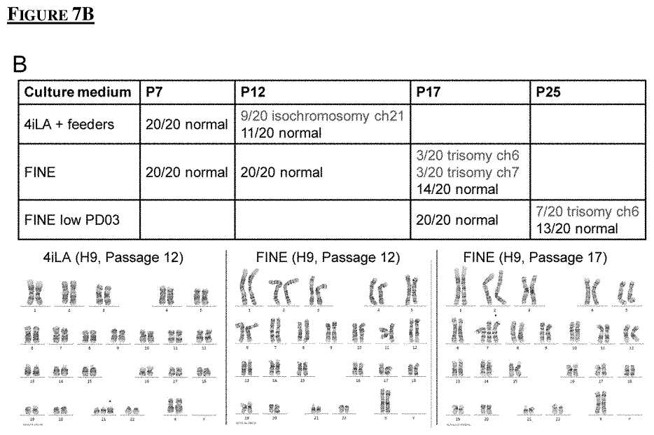

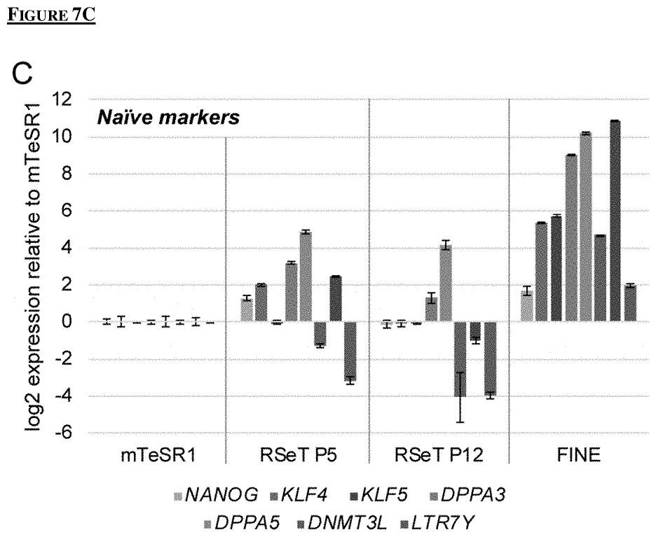

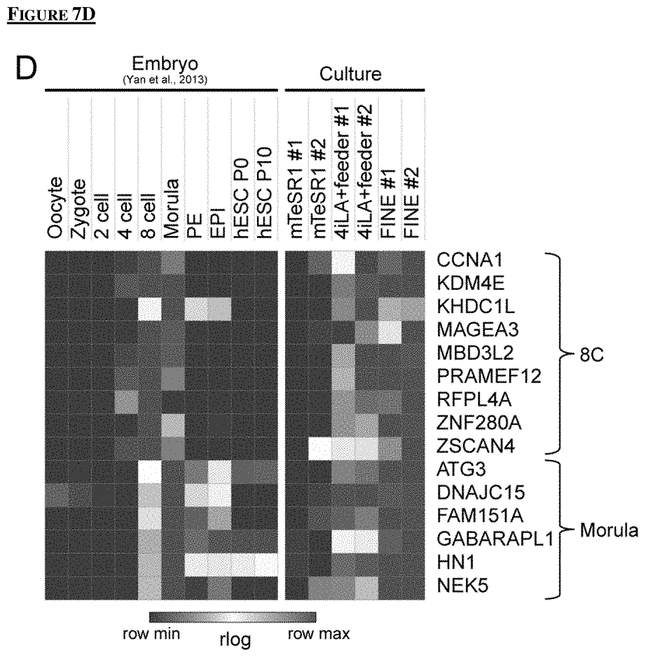

Using a mCherry-expressing plasmid to report transfection efficiency and by co-staining with CD75 to exclude feeders from quantification, we observe more than 5× double-positive cells in FINE than in 4 iLA+feeder cells (FIG. 7A, S5A).

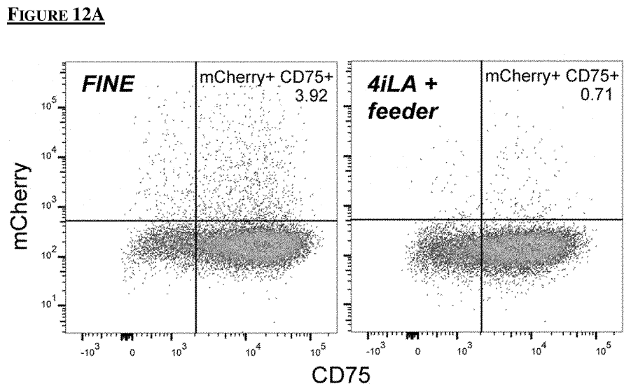

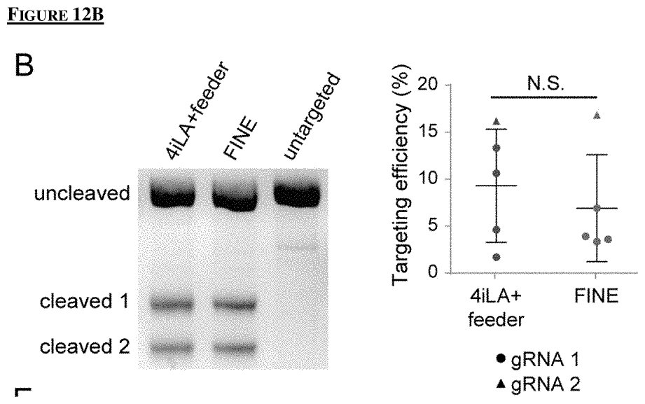

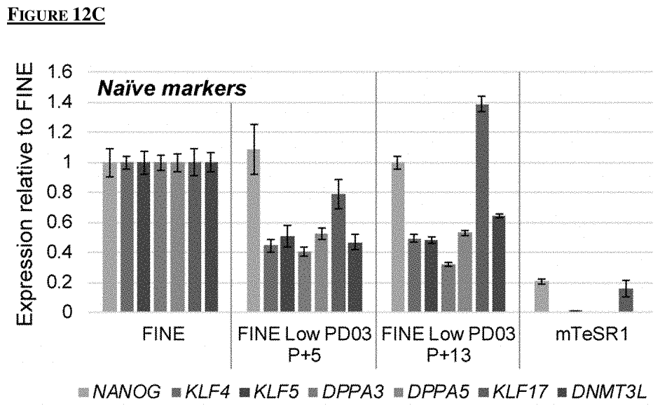

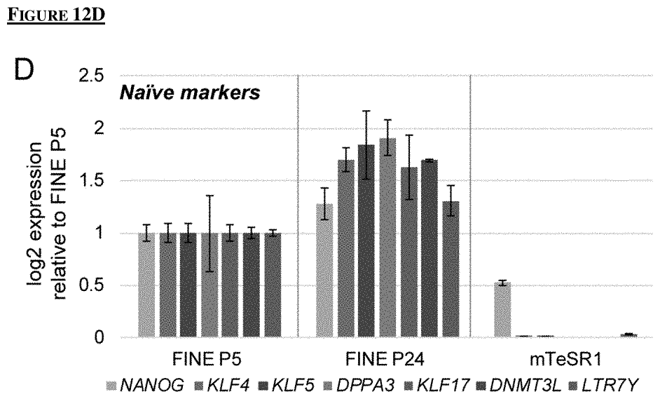

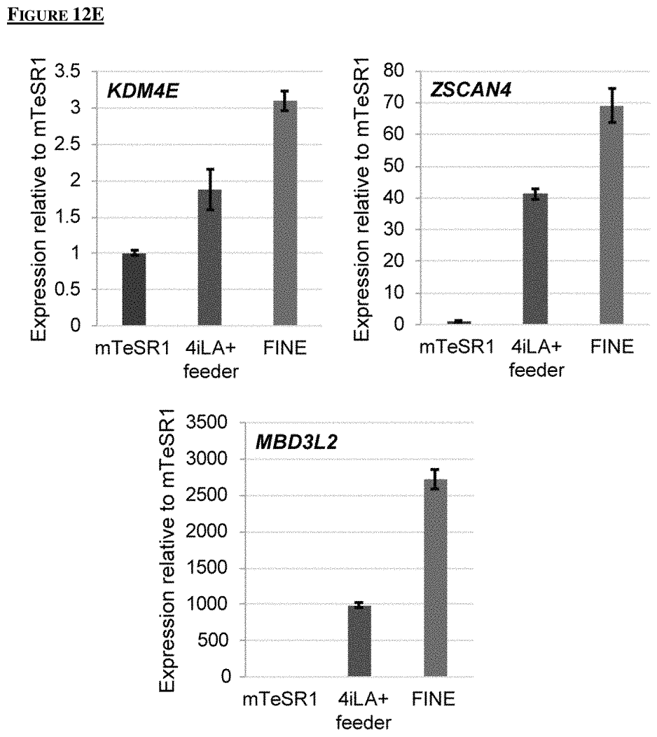

Moreover, we tested ease of genome editing under FINE using the CRISPR-Cas9 system. Using gRNAs targeting human-specific sequences, we observe that hESCs had no significant difference in terms of gene editing efficiency between FINE and 4 iLA+feeder conditions (independent of transfection efficiency, as only positively transfected cells were utilized) (FIG. 12B).

Thus, combined with the general simplicity of handling associated with the absence of feeders, these results indicate that FINE culture allows for easier genetic targeting of naïve hESCs.

One of the main disadvantages of current human naïve culture conditions is its inherent genomic instability (Theunissen et al., 2014). FINE cells are karyotypically normal up to passage 12, indicating that FINE cells are not an artefact of spontaneous genetic abnormalities (FIG. 7B). Importantly, comparative cytogenetic analysis across various passage numbers indicate that FINE cells acquire chromosomal abnormalities at a slower rate than 4 iLA+feeder cells (FIG. 7B), albeit having similar proliferation kinetics (FIG. 9F). To further improve this, we tried lowering the concentration of PD0325901 in FINE, following a recent study that demonstrated enhanced genetic stability upon lower dosage of MEK inhibition (Di Stefano et al., 2018). While this further delayed acquisition of chromosomal abnormalities (FIG. 7B), this had a slight adverse effect on naïve pluripotent marker expression (FIG. 12C). Conversely, extended culture in FINE (P24) does not repress (but actually even slightly increases) naïve marker expression, despite chromosomal defects (FIG. 12D).

We also compared how FINE fares against RSeT, a commercially-available feeder-free media for naïve hESCs based on (Gafni et al., 2013). We find that upregulation of naïve markers is greater and more consistent in FINE (FIG. 7C), including markers that have been reported to be upregulated by RSeT culture. Finally, we also observe that markers associated with earlier stages of development (like the 8-cell stage) are slightly enhanced in FINE compared to 4 iLA on feeder (FIG. 7D, S5E). Taken together, FINE improves on existing naïve culture conditions by offering robust naïve marker expression, including 8-cell stage-specific transcripts, under feeder-free conditions, while improving genomic stability and amenability to gene editing techniques.

### Example 9. Discussion: ERVs as Molecular Landmarks for Cellular States

Here, we exploited the stage-specific transcription of ERVs during embryogenesis to generate an ERV-based LTR7Y fluorescent reporter and demonstrated its utility in an unbiased chemical screen that led us to establishment of a feeder-free naïve medium composition.

Our results and research in mouse ESCs (Macfarlan et al., 2012) indicate that with the help of accurate and sensitive reporters like ERVs, it is possible to isolate a cell state beyond existing in vitro models. Application of a similar strategy can be used for generation of cellular models that corresponds to cells from stages of development other than the blastocyst, and enable further understanding of the requirements for establishment of cellular potency and initiation of cell fate decisions, which is still largely inaccessible to research.

### Example 10. Discussion: Mechanism of Action of Effective Compounds in Fine

The unique ability of FINE to support naïve hESCs in the absence of feeders is endowed by the synergistic action of two compounds: AZD5438 and Dasatinib.

Dasatinib is a broad kinase inhibitor affecting multiple receptor and non-receptor tyrosine kinase families (Li et al., 2010). One of its known targets that play a role in naïve pluripotency is Src, but it must target other additional pathways as other Src inhibitors are unable to sustain feeder-free naïve hESCs (FIG. 4D).

On the other hand, AZD5438 inhibits cyclin-dependent kinases 1 and 2, which largely act in checkpoints of the S and G2 phases of the cell cycle. We have demonstrated before that prolonging these phases of the cell cycle restricts exit from pluripotency in primed hESCs (Gonzales et al., 2015), and AZD5438 might act in this manner to maintain pluripotency in the absence of feeders. Yet, like Dasatinib, replacement of AZD5438 with another CDK1/2/9 inhibitor was not sufficient to sustain FINE cells.

Thus, while beyond the scope of this paper, it is tempting to speculate that these compounds, together or alone, affect unprecedented pathways to induce and maintain feeder-free naivety. Furthermore, several of these compounds' known target pathways have not been studied in the context of naïve pluripotency, and as such, dissecting the mechanisms of these compounds will be an exciting avenue to pursue in the future.

Finally, given that these compounds were discovered in an unbiased screen, it is possible that the combination of AZD5438 and Dasatinib, and perhaps other hits from our screen, may be applicable to endow feeder independence in other culture systems beyond naïve hESCs.

### Example 11. Discussion: Applications of Feeder-free Naïve hESCs

Until now a number of protocols using various cocktails of molecules has been reported to induce naïve states mimicking in vitro human pre-implantation epiblast (Chan et al., 2013; Gafni et al., 2013; Takashima et al., 2014; Theunissen et al., 2014; Ware et al., 2014).

In these studies, establishment of human naïve medium composition was guided by previous knowledge obtained from the mouse. However, species-specific differences exist between mouse and human pluripotency. For example, GSK3 inhibitor is commonly used in naïve cocktails, and in mESCs, acts by elevating levels of Esrrb (Martello et al., 2012). In contrast, ESRRB is not expressed in human pluripotent states (Weinberger et al., 2016) either in vivo (blastocyst's inner cell mass) or in vitro (primed and naïve hESCs), suggesting that GSK3 inhibition might act via different mechanism in human naïve pluripotent culture.

In addition, naïve hESCs hitherto have been dependent on feeders, which introduces a non-defined component and hampers both their acceptance in clinical use and the application of certain technical approaches for dissection of mechanisms behind the state. So far, there are only two feeder-free alternatives for naïve culture of hESCs: first is RSeT medium [based on (Gafni et al., 2013)], which we and others have shown to have divergent transcriptional and epigenetic profiles from best-in-class naïve culture systems (Barakat et al., 2018; Nakamura et al., 2016) (FIG. 7C); second is the protocol from Smith and colleagues (Guo et al., 2017), whose caveats include the requirement for HDAC inhibitors, which are known to increase susceptibility to genomic instability (Eot-Houllier et al., 2009), and the multi-step derivation process that still undergoes temporary culture on feeders for stabilization.

Here, we developed a simple feeder-independent system called FINE that can be used for both the establishment and sustenance of the human naïve state. Conversion to naïve cells in FINE is a one-step process that does not require use of non-defined components.

Therefore, FINE provides a purely chemically-defined xeno-free platform for further dissection of the mechanisms controlling human early development. This is especially useful in dissecting the role of the various small molecules that define human naïve pluripotent culture.

Absence of feeders also allows for unbiased high-throughput screens for identification of novel contributors to the naïve state without the complication of secondary phenotypes from extraneous supporting cells not normally found in embryonic development in vivo. FINE also enables easy genetic targeting of naïve hESCs, not only through the ease of handling due to its feeder-free nature (e.g. removing the requirement for antibiotic-resistant feeders for selection), but also to its inherent amenability to such techniques (FIG. 7A, S5A-B). We also observe that FINE cells acquire chromosomal abnormalities slower (FIG. 7B), suggesting that our system also improves genetic stability compared to its feeder-dependent counterparts. Thus, FINE culture has the potential to be the go-to system for establishment, propagation and examination of human naïve pluripotent cells.

In conclusion, we first identified novel molecules that facilitate feeder independence of naïve hESC culture, which could be useful to guide studies in understanding how fibroblast feeders provide an artificial niche for stem cell culture in vitro. Second and more importantly, through rigorous optimization, we developed a simple chemically-defined xeno-free method of establishing and maintaining naïve hESCs called FINE. This platform offers technical advantages for the mechanistic dissection of naïve identity and will serve as a useful foundation for translational applications of naïve pluripotent stem cells.

### REFERENCES

Araujo, J., and Logothetis, C. (2010). Dasatinib: a potent SRC inhibitor in clinical development for the treatment of solid tumors. Cancer Treat Rev 36, 492-500.

Barakat, T. S., Halbritter, F., Zhang, M., Rendeiro, A. F., Perenthaler, E., Bock, C., and Chambers, I. (2018). Functional Dissection of the Enhancer Repertoire in Human Embryonic Stem Cells. Cell Stem Cell 23, 276-288 e278.

Betschinger, J., Nichols, J., Dietmann, S., Corrin, P. D., Paddison, P. J., and Smith, A. (2013). Exit from Pluripotency Is Gated by Intracellular Redistribution of the bHLH Transcription Factor Tfe3. Cell 153, 335-347.

Brons, I. G., Smithers, L. E., Trotter, M. W., Rugg-Gunn, P., Sun, B., Chuva de Sousa Lopes, S. M., Howlett, S. K., Clarkson, A., Ahrlund-Richter, L., Pedersen, R. A., et al. (2007). Derivation of pluripotent epiblast stem cells from mammalian embryos. Nature 448, 191-195.

Chambers, I., Silva, J., Colby, D., Nichols, J., Nijmeijer, B., Robertson, M., Vrana, J., Jones, K., Grotewold, L., and Smith, A. (2007). Nanog safeguards pluripotency and mediates germline development. Nature 450, 1230-1234.

Chan, Y. S., Goke, J., Ng, J. H., Lu, X., Gonzales, K. A., Tan, C. P., Tng, W. Q., Hong, Z. Z., Lim, Y. S., and Ng, H. H. (2013). Induction of a human pluripotent state with distinct regulatory circuitry that resembles preimplantation epiblast. Cell Stem Cell 13, 663-675.

Di Stefano, B., Ueda, M., Sabri, S., Brumbaugh, J., Huebner, A. J., Sahakyan, A., Clement, K., Clowers, K. J., Erickson, A. R., Shioda, K., et al. (2018). Reduced MEK inhibition preserves genomic stability in naïve human embryonic stem cells. Nat Methods 15, 732-740.

Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinformatics 29, 15-21.

Eot-Houllier, G., Fulcrand, G., Magnaghi-Jaulin, L., and Jaulin, C. (2009). Histone deacetylase inhibitors and genomic instability. Cancer Lett 274, 169-176.

Evans, M. J., and Kaufman, M. H. (1981). Establishment in culture of pluripotential cells from mouse embryos. Nature 292, 154-156.

Gafni, O., Weinberger, L., Mansour, A. A., Manor, Y. S., Chomsky, E., Ben-Yosef, D., Kalma, Y., Viukov, S., Maza, I., Zviran, A., et al. (2013). Derivation of novel human ground state naïve pluripotent stem cells. Nature 504, 282-286.

Gardner, R. L. (1998). Contributions of blastocyst micromanipulation to the study of mammalian development. Bioessays 20, 168-180.

Gardner, R. L., and Beddington, R. S. (1988). Multi-lineage ‘stem’ cells in the mammalian embryo. J Cell Sci Suppl 10, 11-27.

Gentleman, R. C., Carey, V. J., Bates, D. M., Bolstad, B., Dettling, M., Dudoit, S., Ellis, B., Gautier, L., Ge, Y., Gentry, J., et al. (2004). Bioconductor: open software development for computational biology and bioinformatics. Genome Biol 5, R80.

Goke, J., Lu, X., Chan, Y. S., Ng, H. H., Ly, L. H., Sachs, F., and Szczerbinska, I. (2015). Dynamic transcription of distinct classes of endogenous retroviral elements marks specific populations of early human embryonic cells. Cell Stem Cell 16, 135-141.

Gonzales, K. A., Liang, H., Lim, Y. S., Chan, Y. S., Yeo, J. C., Tan, C. P., Gao, B., Le, B., Tan, Z. Y., Low, K. Y., et al. (2015). Deterministic Restriction on Pluripotent State Dissolution by Cell-Cycle Pathways. Cell 162, 564-579.

Grow, E .J., Flynn, R. A., Chavez, S. L., Bayless, N. L., Wossidlo, M., Wesche, D. J., Martin, L., Ware, C. B., Blish, C. A., Chang, H. Y., et al. (2015). Intrinsic retroviral reactivation in human preimplantation embryos and pluripotent cells. Nature 522, 221-225.

Guo, G., von Meyenn, F., Rostovskaya, M., Clarke, J., Dietmann, S., Baker, D., Sahakyan, A., Myers, S., Bertone, P., Reik, W., et al. (2017). Epigenetic resetting of human pluripotency. Development 144, 2748-2763.

Hayashi, K., Lopes, S. M., Tang, F., and Surani, M. A. (2008). Dynamic equilibrium and heterogeneity of mouse pluripotent stem cells with distinct functional and epigenetic states. Cell Stem Cell 3, 391-401.

Kumar, P., Goh, G., Wongphayak, S., Moreau, D., and Bard, F. (2013). ScreenSifter: analysis and visualization of RNAi screening data. BMC Bioinformatics 14, 290.

Lawrence, M., Huber, W., Pages, H., Aboyoun, P., Carlson, M., Gentleman, R., Morgan, M. T., and Carey, V. J. (2013). Software for computing and annotating genomic ranges. PLoS Comput Biol 9, e1003118.

Li, J., Rix, U., Fang, B., Bai, Y., Edwards, A., Colinge, J., Bennett, K. L., Gao, J., Song, L., Eschrich, S., et al. (2010). A chemical and phosphoproteomic characterization of dasatinib action in lung cancer. Nat Chem Biol 6, 291-299.

Lin, H., Gupta, V., Vermilyea, M. D., Falciani, F., Lee, J. T., O'Neill, L. P., and Turner, B. M. (2007). Dosage compensation in the mouse balances up-regulation and silencing of X-linked genes. PLoS Biol 5, e326.

Love, M. I., Huber, W., and Anders, S. (2014). Moderated estimation of fold change and dispersion for RNA-seq data with DESeq2. Genome Biol 15, 550.

Lu, X., Sachs, F., Ramsay, L., Jacques, P. E., Goke, J., Bourque, G., and Ng, H. H. (2014). The retrovirus HERVH is a long noncoding RNA required for human embryonic stem cell identity. Nat Struct Mol Biol 21, 423-425.

Macfarlan, T. S., Gifford, W. D., Driscoll, S., Lettieri, K., Rowe, H. M., Bonanomi, D., Firth, A., Singer, O., Trono, D., and Pfaff, S. L. (2012). Embryonic stem cell potency fluctuates with endogenous retrovirus activity. Nature 487, 57-63.

Martello, G., Sugimoto, T., Diamanti, E., Joshi, A., Hannah, R., Ohtsuka, S., Gottgens, B., Niwa, H., and Smith, A. (2012). Esrrb is a pivotal target of the gsk3/tcf3 axis regulating embryonic stem cell self-renewal. Cell Stem Cell 11, 491-504.

Martin, G. R. (1981). Isolation of a pluripotent cell line from early mouse embryos cultured in medium conditioned by teratocarcinoma stem cells. Proc Natl Acad Sci U S A 78, 7634-7638.

Nakamura, T., Okamoto, I., Sasaki, K., Yabuta, Y., Iwatani, C., Tsuchiya, H., Seita, Y., Nakamura, S., Yamamoto, T., and Saitou, M. (2016). A developmental coordinate of pluripotency among mice, monkeys and humans. Nature 537, 57-62.

Nichols, J., and Smith, A. (2009). Naïve and primed pluripotent states. Cell Stem Cell 4, 487-492.

Nishizawa, M., Chonabayashi, K., Nomura, M., Tanaka, A., Nakamura, M., Inagaki, A., Nishikawa, M., Takei, I., Oishi, A., Tanabe, K., et al. (2016). Epigenetic Variation between Human Induced Pluripotent Stem Cell Lines Is an Indicator of Differentiation Capacity. Cell Stem Cell 19, 341-354.

Niwa, H., Ogawa, K., Shimosato, D., and Adachi, K. (2009). A parallel circuit of LIF signalling pathways maintains pluripotency of mouse ES cells. Nature 460, 118-122.

R Development Core Team (2014). R: A language and environment for statistical computing (Vienna, Austria: R Foundation for Statistical Computing).

Ran, F. A., Hsu, P. D., Wright, J., Agarwala, V., Scott, D. A., and Zhang, F. (2013). Genome engineering using the CRISPR-Cas9 system. Nat Protoc 8, 2281-2308.

Reubinoff, B. E., Pera, M. F., Fong, C. Y., Trounson, A., and Bongso, A. (2000). Embryonic stem cell lines from human blastocysts: somatic differentiation in vitro. Nat Biotechnol 18, 399-404.

Sahakyan, A., Kim, R., Chronis, C., Sabri, S., Bonora, G., Theunissen, T. W., Kuoy, E., Langerman, J., Clark, A. T., Jaenisch, R., et al. (2017). Human Naïve Pluripotent Stem Cells Model X Chromosome Dampening and X Inactivation. Cell Stem Cell 20, 87-101.

Takashima, Y., Guo, G., Loos, R., Nichols, J., Ficz, G., Krueger, F., Oxley, D., Santos, F., Clarke, J., Mansfield, W., et al. (2014). Resetting transcription factor control circuitry toward ground-state pluripotency in human. Cell 158, 1254-1269.

Tesar, P. J., Chenoweth, J. G., Brook, F. A., Davies, T. J., Evans, E. P., Mack, D. L., Gardner, R. L., and McKay, R. D. (2007). New cell lines from mouse epiblast share defining features with human embryonic stem cells. Nature 448, 196-199.

Theunissen, T. W., Friedli, M., He, Y., Planet, E., O'Neil, R. C., Markoulaki, S., Pontis, J., Wang, H., Iouranova, A., Imbeault, M., et al. (2016). Molecular Criteria for Defining the Naïve Human Pluripotent State. Cell Stem Cell 19, 502-515.

Theunissen, T. W., Powell, B. E., Wang, H., Mitalipova, M., Faddah, D. A., Reddy, J., Fan, Z. P., Maetzel, D., Ganz, K., Shi, L., et al. (2014). Systematic identification of culture conditions for induction and maintenance of naïve human pluripotency. Cell Stem Cell 15, 471-487.

Thomson, J. A., Itskovitz-Eldor, J., Shapiro, S. S., Waknitz, M. A., Swiergiel, J. J., Marshall, V. S., and Jones, J. M. (1998). Embryonic stem cell lines derived from human blastocysts. Science 282, 1145-1147.

Torres-Padilla, M. E., and Chambers, I. (2014). Transcription factor heterogeneity in pluripotent stem cells: a stochastic advantage. Development 141, 2173-2181.

Tosolini, M., Brochard, V., Adenot, P., Chebrout, M., Grillo, G., Navia, V., Beaujean, N., Francastel, C., Bonnet-Gamier, A., and Jouneau, A. (2018). Contrasting epigenetic states of heterochromatin in the different types of mouse pluripotent stem cells. Sci Rep 8, 5776.

van den Berg, D. L., Zhang, W., Yates, A., Engelen, E., Takacs, K., Berstarosti, K., Demmers, J., Chambers, I., and Poot, R. A. (2008). Estrogen-related receptor beta interacts with Oct4 to positively regulate Nanog gene expression. Mol Cell Biol 28, 5986-5995.

Ware, C. B. (2017). Concise Review: Lessons from Naïve Human Pluripotent Cells. Stem Cells 35, 35-41.

Ware, C. B., Nelson, A. M., Mecham, B., Hesson, J., Zhou, W., Jonlin, E. C., Jimenez-Caliani, A. J., Deng, X., Cavanaugh, C., Cook, S., et al. (2014). Derivation of naïve human embryonic stem cells. Proc Natl Acad Sci U S A 111, 4484-4489.

Weinberger, L., Ayyash, M., Novershtern, N., and Hanna, J. H. (2016). Dynamic stem cell states: naïve to primed pluripotency in rodents and humans. Nat Rev Mol Cell Biol 17, 155-169.

Wickham, H (2016). Ggplot2—elegant graphics for data analysis.

Xue, Z., Huang, K., Cai, C., Cai, L., Jiang, C. Y., Feng, Y., Liu, Z., Zeng, Q., Cheng, L., Sun, Y. E., et al. (2013). Genetic programs in human and mouse early embryos revealed by single-cell RNA sequencing. Nature 500, 593-597.

Yan, L., Yang, M., Guo, H., Yang, L., Wu, J., Li, R., Liu, P., Lian, Y., Zheng, X., Yan, J., et al. (2013). Single-cell RNA-Seq profiling of human preimplantation embryos and embryonic stem cells. Nat Struct Mol Biol 20, 1131-1139.

Ying, Q. L., Wray, J., Nichols, J., Batlle-Morera, L., Doble, B., Woodgett, J., Cohen, P., and Smith, A. (2008). The ground state of embryonic stem cell self-renewal. Nature 453, 519-523.

Zernicka-Goetz, M., Morris, S. A., and Bruce, A. W. (2009). Making a firm decision: multifaceted regulation of cell fate in the early mouse embryo. Nat Rev Genet 10, 467-477.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A cell culture medium comprising a CDK1/2/9 inhibitor and a Bcr-Abl/Src kinase inhibitor.

2. The cell culture medium according to claim 1, in which the CDK1/2/9 inhibitor comprises AZD5438 (4-[2-Methyl-1-(1-methylethyl)-1H-imidazol-5-yl]-N-[4-(methylsulfonyl) phenyl]-2-pyrimidinamine, AZD) or in which the Bcr-Abl/Src kinase inhibitor comprises Dasatinib (N-(2-chloro-6-methylphenyl)-2-[[6-[4-(2-hydroxyethyl)-1-piperazinyl]-2-methyl-4-pyrimidinyl]amino]-5-thiazole carboxamide monohydrate, DASA).

3. The cell culture medium according to claim 1, in which the cell culture medium comprises AZD5438 and Dasatinib, each independently at a concentration of 0.1 μM or more, such as 1 μM to 0.5 μM, preferably 0.1 μM.

4. The cell culture medium according to claim 1, in which the cell culture medium comprises one or more of: SB590885 ((NE)-N-[5-[2-[4-[2-(dimethylamino) ethoxy]phenyl]-5-pyridin-4-yl-1H-imidazol-4-yl]-2,3-dihydroinden-1-ylidene]hydroxylamine); PD0325901 (N-[(2R)-2,3-dihydroxypropoxy]-3,4-difluoro-2-(2-fluoro-4-iodoanilino)benzamide); and Y-27632 (4-[(1R)-1-aminoethyl]-N-pyridin-4-ylcyclohexane-1-carboxamide) such as 0.1 to 2.5 μM, preferably 0.5 μM of SB590885, 0.2 to 10 μM, preferably 1 μM of PD0325901 or 5 to 20 μM, preferably 10 μM of Y-27632.

5. The cell culture medium according to claim 1, in which the cell culture medium comprises: 5 to 20 m/ml of recombinant human LIF (UniProtKB-P15018); 0.2 to 10 μM, preferably 1 μM of PD0325901; 0.1 to 2.5 μM, preferably 0.5 μM of SB590885; 0.1 to 2.5 μM, preferably 1 82 M of WH4-023; 5 to 20 μM, preferably 10 μM of Y-27632; and 5 to 20 ng/ml, preferably 10 ng/ml of Activin A (UniProtKB-P08476).

6. The cell culture medium according to claim 1, in which the cell culture medium comprises: DMEM/F12 (Invitrogen; 11320), Neurobasal (Invitrogen; 21103), N2 supplement (Invitrogen; 17502048) (100× dilution), B27 supplement (Invitrogen; 17504044) (50× dilution), 2 mM L-glutamine, 1% non-essential amino acids, 0.1 mM β-mercaptoethanol, 1% penicillin-streptomycin, 50 μg/ml BSA, supplemented with 10 μg/mL recombinant human LIF, 1 μM PD0325901, 0.5 μM SB590885, 1 μM WH4-023, 10 μM Y-27632 and 10 ng/mL Activin A, preferably in which the cell culture medium comprises a 1:1 ratio of F12 DMEM (STEMCELL Technologies) and Neurobasal media (Gibco), 1× N2 supplement (Gibco) and 1× B2 supplement (Gibco), 1× L-Glutamine (Gibco), 1× Non-essential amino acids (Gibco), 0.1 mM of B-mercaptoethanol (Sigma) and 62.5 ng/ml of bovine serum albumin (BSA, Sigma).

7. The cell culture medium according to claim 1, in which the cell culture medium is capable of maintaining or increasing pluripotency in a cell cultured in the cell culture medium in the absence of co-culture such as feeder cells.

8. The cell culture medium according to any preceding claim 7, in which the pluripotency comprises expression of a naïve pluripotent stem cell marker selected from the group consisting of: CD130 (Gene ID: 3572), CD75 (Gene ID: 6480), DNMT3L (Gene ID: 29947), DPPAS (Gene ID: 340168), KLFS (Gene ID: 688), TFCP2L1 (Gene ID: 29842), KLF4 (Gene ID: 9314), DPPA3 (Gene ID: 359787), NANOG (Gene ID: 79923), KLF17 (Gene ID: 128209), POU5F1 (Gene ID: 5460) and PRDM14 (Gene ID: 63978).

9. The cell culture medium according to claim 1, in which the cell culture medium is capable of maintaining or increasing pluripotency in a cell cultured for 5 or more passages, such as 8 or more passages.

10. The cell culture medium according to claim 1, in which the cell culture medium is capable of decreasing the expression of a primed pluripotent stem cell marker such as ZIC2 (Gene ID: 7546) and B3GAT1 (Gene ID: 27087) in a cell cultured in the cell culture medium.

11. A method of culturing a cell in a cell culture medium according to claim 1.

12. The method according to claim 11, in which the method is capable of maintaining or increasing the expression of a naïve pluripotent stem cell marker in the cell.

13. The method according to claim 11, in which the method does not include co-culture with feeder cells.

14. The method according to claim 11, in which the method comprises culturing the cell for 5 or more passages, such as 8 or more passages.

15. The method according to claim 11, in which the cell comprises a naïve pluripotent stem cell, preferably a mammalian naïve pluripotent stem cell, such as a human naïve pluripotent stem cell.

16. The method according to claim 11, in which the method is capable of:
(a) maintaining the naïve pluripotent stem cell in a naïve state; and/or
(b) maintaining the survival of a naïve pluripotent stem cell
preferably after at least 5 passages, preferably after at least 8 passages.

17. The method according to any preceding claim 11, in which the cell comprises a primed pluripotent stem cell, preferably a mammalian primed pluripotent stem cell, such as a human primed pluripotent stem cell, in which the method re-programs the primed pluripotent stem cell into a naïve pluripotent stem cell.

18. The method according to claim 11, in which the cell comprises a somatic cell, preferably a mammalian somatic cell, such as a human somatic cell, in which the method re-programs the somatic cell into a naïve pluripotent stem cell, and in which the method preferably further comprises up-regulating the expression of Oct4 (Pou5f1), Sox2, Klf4 and c-Myc in the somatic cell.

19. A method of propagation of a naïve pluripotent stem cell, the method comprising culturing the naïve pluripotent stem cell in a cell culture medium according to claim 1.

20. A method of re-programming a somatic cell or a primed pluripotent stem cell into a naïve pluripotent stem cell, the method comprising culturing the primed pluripotent stem cell in a cell culture medium according to claim 1.

21-22. (canceled)
